(19) United States
(12) Patent Application Publication
(10) Pub. No.: US 2008/0161309 A1
(43) Pub. Date: Jul. 3, 2008

(54) Novel Compounds

(76) Inventors: Paul Vincent Fish, (US); Malcolm Christian MacKenny, (US); Alan Stobie, (US); Florian Wakenhut, (US); Gavin Alistair Whitlock, (US)

Correspondence Address: PHARMACIA CORPORATION;GLOBAL PATENT DEPARTMENT, POST OFFICE BOX 1027, ST. LOUIS, MO, 63006, US

(21) Appl. No.: 12/034,945

(22) Filed: Feb. 21, 2008

Related U.S. Application Data
(63) Continuation of application No. 11/117,896, filed on Apr. 28, 2005, now abandoned.
(60) Provisional application No. 60/576,337, filed on Jun. 2, 2004.

(30) Foreign Application Priority Data
Apr. 30, 2004 (GB) 0409744.0

(51) Int. Cl. A61K 31/5375; C07D 295/00; A61P 13/02; A61P 15/00; A61P 25/22; A61P 25/00

(52) U.S. Cl. 514/239.2; 544/174

(57) ABSTRACT

The present invention provides compounds of Formula I

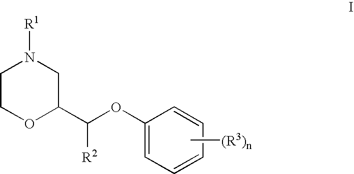

wherein R¹, R², R³, and n have any of the values defined in the specification, and pharmaceutically acceptable salts thereof, that are useful as agents in the treatment of conditions including urinary disorders, pain, premature ejaculation, ADHD and fibromyalgia. Also provided are pharmaceutical compositions comprising one or more compounds of Formula I.

This application claims priority to U.S. Non-Provisional application Ser. No. 11/117,896 filed Apr. 28, 2005 which claims priority to U.S. Provisional Application No. 60/576,337 filed Jun. 2, 2004 and GB Application Serial No. 0409744.0 filed Apr. 30, 2004.

### BACKGROUND OF THE INVENTION

The monoamines norepinephrine (noradrenaline) and serotonin (5-HT) have a variety of nervous system effects as neurotransmitters. These monoamines are taken up by neurons after being released into the synaptic cleft. Norepinephrine and serotonin are taken up from the synaptic cleft by their respective norepinephrine and serotonin transporters.

Drugs that inhibit the norepinephrine and/or serotonin transporters have been used to treat a variety of nervous system disorders. For example, the serotonin transporter inhibitor fluoxetine has been found to be useful in the treatment of depression, and other central nervous system disorders. The norepinephrine reuptake inhibitor atomoxetine has been approved for the treatment of attention deficit hyperactivity disorder (ADHD). In addition, the norepinephrine and serotonin transporter inhibitor milnacipran is being developed for the treatment of fibromyalgia.

There is an ongoing need in the art for compounds that are norepinephrine transporter inhibitors, serotonin transporter inhibitors, and that inhibit both norepinephrine and serotonin transporters, for the treatment of disorders including ADHD, urinary incontinence disorders, depression, generalised anxiety disorder, fibromyalgia, and pain.

### SUMMARY OF THE INVENTION

This invention relates to novel morpholine compounds which inhibit monoamine re-uptake, to processes for their preparation, to pharmaceutical compositions containing them and to their use in medicine.

The compounds of the invention exhibit activity as both serotonin and noradrenaline re-uptake inhibitors and therefore have utility in a variety of therapeutic areas. For example, the compounds of the invention are of use in the treatment of disorders in which the regulation of monoamine transporter function is implicated; more particularly disorders in which inhibition of re-uptake of serotonin or noradrenaline is implicated; and especially disorders in which inhibition of reuptake of both serotonin and noradrenaline is implicated, such as urinary incontinence.

According to a first aspect, the invention provides a use of a compound of Formula I, as defined below in Integers 1 to 10.

Integer 1: Use of a compound of Formula (I) in the manufacture of a medicament for the treatment of a disorder in mammals in which the regulation of monoamine transporter function is implicated, wherein the disorder is selected from urinary disorders, pain, premature ejaculation, ADHD and fibromyalgia, and the compound of Formula (I) is:

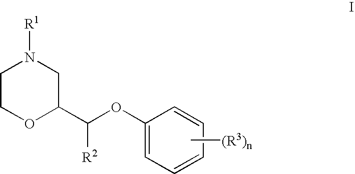

and pharmaceutically and/or veterinarily acceptable derivatives thereof, wherein:
R¹ is H or C₁₋₆alkyl;
R² is aryl, het, (CH₂)_{z}aryl or R⁴, wherein each of the aryl, het and R⁴ groups is optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄-alkoxy-C₁₋₄alkoxy, SCF₃, C₁₋₆alkyl-SO₂—, C₁₋₄alkyl-S—C₁₋₄alkyl, C₁₋₄alkyl-S—, C₁₋₄alkylNR¹⁰R¹¹ and NR¹⁰R¹¹; each R³ is independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂, C₁₋₄alkyl-S—C₁₋₄alkyl, C₁₋₄alkyl-S—, C₁₋₆alkylNR¹⁰R¹¹ and NR¹⁰R¹¹;
n is an integer between 0 and 4, wherein when n is 2, the two R³ groups together with the phenyl ring to which they are attached may represent a benzofused bicyclic ring comprising a phenyl group fused to a 5- or 6-membered carbocyclic group, or a phenyl group fused to a 5- or 6-membered heterocyclic group containing at least one N, O or S heteroatom;
R⁴ is a phenyl group fused to a 5- or 6-membered carbocyclic group, or a phenyl group fused to a 5- or 6-membered heterocyclic group containing at least one N, O or S heteroatom;
R¹⁰ and R¹¹ are the same or different and are independently H or C₁₋₄alkyl;
y is 1 or 2;
z is an integer from 1 to 3; aryl is phenyl, naphthyl, anthracyl or phenanthryl; and
het is an aromatic or non-aromatic 4-, 5- or 6-membered heterocycle which contains at least one N, O or S heteroatom, optionally fused to a 5- or 6-membered carbocyclic group or a second 4-; 5- or 6-membered heterocycle which contains at least one N, O or S heteroatom;
provided that the compound is not 2-[(2-ethoxyphenoxy)(phenyl)methyl]morpholine.

Integer 2: Use of a compound according to Integer 1, wherein R¹ is H.

Integer 3: Use of a compound according to Integer 1 or Integer 2, wherein R² is aryl or het, each optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₁₆alkyl, C₁₋₄alkoxy-C₁₋₄alkoxy, SCF₃, C₁₋₆alkyl-SO₂—, C₁₋₄alkyl-S—C₁₋₄alkyl, C₁₋₄alkyl-S—, C₁₋₄-alkylNR¹⁰R¹¹ and NR¹⁰R¹¹.

Integer 4: Use of a compound according to Integer 3, wherein R² is phenyl, pyridinyl or thiazole, wherein each of the phenyl, pyridinyl and thiazole groups is optionally substituted by at least one substituent independently selected from C₁₋₆alkyl, C₁₋₆-alkoxy, OH, halo, CF₃, OCF₃, OCHF₂. O(CH₂)_{y}CF₃, CN, CONH₂, CON(H)C₁₋₆alkyl, CON(C₁₋₆alkyl)₂, hydroxy-C₁₋₁₆alkyl, C₁₋₄alkoxy-C₁₋₆alkyl, C₁₋₄alkoxy-C₁₋₄alkoxy, SCF₃, C₁₋₆alkyl-SO₂—, C₁₋₄alkyl-S—C₁₋₄alkyl, C₁₋₄alkyl-S—, C₁₋₄alkylNR¹⁰R¹¹ and NR¹⁰R¹¹.

Integer 5: Use of a compound according to Integer 4, wherein R² is phenyl.

Integer 6: Use of a compound according to any of Integers 1 to 5, wherein the optional substituents for R² are selected from C₁₋₆alkyl, C₁₋₆alkoxy, OH, halo, CF₃, OCF₃, OCHF₂, CN and C₁₋₄alkoxy-C₁₋₆alkyl.

Integer 7: Use of a compound according to any of Integers 1 to 6, wherein each R³ is independently selected from C₁₋₆alkyl, C₁₋₆alkoxy, OH, halo, CF₃, OCF₃, OCHF₂, CN and C₁₋₄alkoxy-C₁₋₆alkyl, or, when n is 2, the two R³ groups together with the phenyl ring to which they are attached may represent a benzofused bicyclic ring comprising a phenyl group fused to a 5- or 6-membered carbocyclic group, or a phenyl group fused to a 5- or 6-membered heterocyclic group containing at least one N, O or S heteroatom.

Integer 8: Use of a compound according to Integer 7, wherein each R³ is independently selected from C₁₋₆alkyl, C₁₋₆alkoxy, OH, halo, CF₃, OCF₃, OCHF₂, CN and C₁₋₄alkoxy-C₁₋₆alkyl.

Integer 9: Use of a compound according to Integer 8, wherein each R³ is independently selected from C₁₋₃alkyl, C₁₋₃alkoxy, OH, F, Cl, CF₃, OCF₃, OCHF₂, CN and C₁₋₃alkoxy-C₁₋₃alkyl.

Integer 10: Use of a compound according to any of Integers 1 to 9, wherein n is 1, 2 or 3.

Integer 11: Use of a compound according to Integer 10, wherein n is 2 or 3.

According to a second aspect of the invention, there is provided a method of treatment of urinary disorders, pain, premature ejaculation, ADHD or fibromyalgia, which comprises administering a therapeutically effective amount of a compound of Formula I as defined in any of Integers 1 to 11 to a mammalian patient in need of such treatment.

According to a third aspect of the invention, there is provided a process for the preparation of a compound of Formula I as defined in any of Integers 1 to 11, the process including either (i) reacting a compound of formula VIII n:

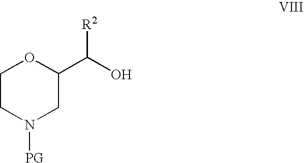

wherein PG is a suitable protecting group, with a phenol compound of formula (R³)ₙPhOH under suitable conditions, followed by deprotection as necessary; or
(ii) cyclising a compound of formula XVII:

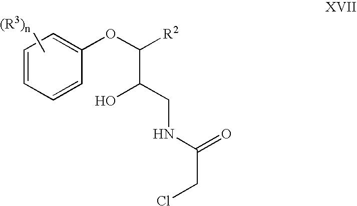

to provide a compound of formula XVIII

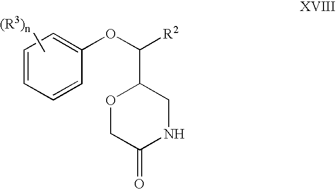

followed by removal of the carbonyl oxygen (═O) from the morpholinone group.

According to a fourth aspect of the invention, there is a provided a compound of Formula I:

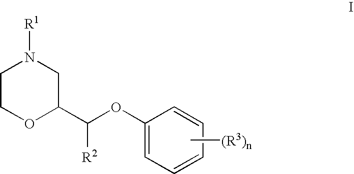

or a pharmaceutically acceptable salt thereof, wherein:
R¹ is H or C₁₋₆alkyl;
R² is aryl, het, (CH₂)₂aryl or R⁴, wherein each of the aryl, het and R⁴ groups is optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₄alkyl, C₁₋₄alkoxy-C₁₋₄alkoxy, SCF₃, C₁₋₆alkyl-SO₂—, C₁₋₄alkyl-S—C₁₋₄alkyl, C₁₋₄alkyl-S—, C₁₋₄alkylNR¹⁰R¹¹ and NR¹⁰R¹¹;
each R³ is independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂, C₁₋₄alkyl-S—C₁₋₄alkyl, C₁₋₄alkyl-S—, C₁₋₄alkylNR¹⁰R¹¹ and NR¹⁰R¹¹;
n is an integer between 0 and 4, wherein when n is 2, the two R³ groups together with the phenyl ring to which they are attached may represent a benzofused bicyclic ring comprising a phenyl group fused to a 5- or 6-membered carbocyclic group, or a phenyl group fused to a 5- or 6-membered heterocyclic group containing at least one N, O or S heteroatom;
R⁴ is a phenyl group fused to a 5- or 6-membered carbocyclic group, or a phenyl group fused to a 5- or 6-membered heterocyclic group containing at least one N, O or S heteroatom;
R¹⁰ and R¹¹ are the same or different and are independently H or C₁₋₄alkyl;
y is 1 or 2;
z is an integer from 1 to 3;
aryl is phenyl, naphthyl, anthracyl or phenanthryl; and
   het is an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom; provided that the compound is not 2-[(2-ethoxyphenoxy)(phenyl)methyl]morpholine.

According to a fifth aspect of the invention, there is provided a compound of Formula Ia:

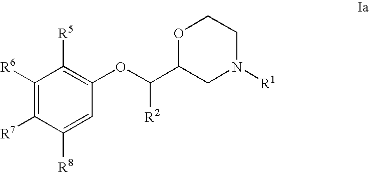

and pharmaceutically and/or veterinarily acceptable derivatives thereof, wherein:
R¹, R², R⁴, R¹⁰, R¹¹, y, z, aryl and het are as defined above in any of Integers 1 to 10 in respect of Formula I;
R⁵ is C₁₋₆alkyl, C₁₋₆alkoxy, halo, CF₃, OCF₃, OCHF₂, O(CH₂)_{y}CF₃, CN, hydroxy-C₁₋₆alkyl, C₁₋₄alkoxy-C₁₋₆alkyl, C₁₋₄alkoxy-C₁₋₄alkoxy, SCF₃, C₁₋₆alkyl-SO₂—, C₁₋₄alkyl-S—C₁₋₄alkyl or C₁₋₄alkyl-S—; and
R⁶, R⁷, and R⁸ are each independently selected from H, C₁₋₆alkyl, C₁₋₆alkoxy, halo, CF₃, OCF₃, OCHF₂. O(CH₂)_{y}CF₃, CN, hydroxy-C₁₋₆alkyl, C₁₋₄alkoxy-C₁₋₆alkyl, C₁₋₄alkoxy-C₁₋₄alkoxy, SCF₃, C₁₋₆alkyl-SO₂—, C₁₋₄alkyl-S—C₁₋₄alkyl or C₁₋₄alkyl-S—;
or two of R⁶, R⁷, or R⁸ together with the phenyl ring to which they are attached may represent a benzofused bicyclic ring comprising a phenyl group fused to a 5- or 6-membered carbocyclic group, or a phenyl group fused to a 5- or 6-membered heterocyclic group containing at least one N, O or S heteroatom,
Provided that at least one of R⁶, R⁷, or R⁸ is not H.

In certain embodiments of the fourth aspect of the invention, R⁵ is C₁₋₆alkyl, C₁₋₆alkoxy, halo, CF₃, OCF₃, OCHF₂, CN or C₁₋₄alkoxy-C₁₋₆alkyl.

In further embodiments, R⁶, R⁷, and R⁸ are each independently selected from H, C₁₋₆alkyl, C₁₋₆alkoxy, halo, CF₃, OCF₃, OCHF₂, CN and C₁₋₄alkoxy-C₁₋₆alkyl. Of course, the invention specifically includes compounds which have the limited definition of R⁵ as defined in the preceding paragraph, together with the limited definitions of R⁶, R⁷ and R⁸ as defined in this paragraph.

Still further embodiments of the fifth aspect of the invention include compounds where R¹ is H. Again, such compounds may also include the more limited definitions of R⁵ and/or R⁶, R⁷ and R₈ as defined in the preceding two paragraphs.

In yet further embodiments, there is provided a compound according to the fifth aspect of the invention, wherein:

R¹ is H;

R² is phenyl, optionally substituted by at least one substituent selected from C₁₋₆alkyl, C₁₋₆alkoxy, OH, halo, CF₃, OCF₃, OCHF₂ and CN;

R⁵ is C₁₋₆alkyl, C₁₋₆alkoxy, OCF₃ or OCHF₂; and

R⁶, R⁷, and R⁸ are each independently selected from H and halo

Specific example compounds within the scope of the fifth aspect of the invention include:
2-[(4-chloro-2-ethoxyphenoxy)(phenyl)methyl]morpholine;
2-[(4-chloro-2-methoxyphenoxy)(phenyl)methyl]morpholine;
2-[[4-chloro-2-(difluoromethoxy)phenoxy](phenyl)methyl]morpholine;
2-[(4-chloro-2-methoxyphenoxy)(phenyl)methyl]morpholine;
2-[(4-chloro-2-ethoxyphenoxy)(phenyl)methyl]morpholine;
2-[(3-chloro-2-ethoxyphenoxy)(phenyl)methyl]morpholine;
2-[(4-chloro-2-fluorophenoxy)(phenyl)methyl]morpholine;
2-[(2,3-difluorophenoxy)(phenyl)methyl]morpholine;
2-[(4-chloro-2-methylphenoxy)(phenyl)methyl]morpholine;
2-[(2,4-difluorophenoxy)(phenyl)methyl]morpholine;
2-[(3-chloro-2-fluorophenoxy)(phenyl)methyl]morpholine;
2-[(2-chloro-4-fluorophenoxy)(phenyl)methyl]morpholine;
2-[[4-chloro-2-(trifluoromethoxy)phenoxy](phenyl)methyl]morpholine;
2-[(2,3-dichlorophenoxy)(phenyl)methyl]morpholine;
2-[(2,4-dichlorophenoxy)(phenyl)methyl]morpholine;
5-chloro-2-[morpholin-2-yl(phenyl)methoxy]benzonitrile;
3-methoxy-4-[morpholin-2-yl(phenyl)methoxy]benzonitrile;
8-[morpholin-2-yl(phenyl)methoxy]quinoline;
2-[(3-chloro-2-methoxyphenoxy)(phenyl)methyl]morpholine;
2-[(4-fluoro-2-methoxyphenoxy)(phenyl)methyl]morpholine;
2-{phenyl[3-(trifluoromethoxy)phenoxy]methyl}morpholine;
2-[[4-chloro-2-(trifluoromethoxy)phenoxy](phenyl)methyl]morpholine;
2-[(4-fluoro-2-methylphenoxy)(phenyl)methyl]morpholine;
3-chloro-4-{[morpholin-2-yl(phenyl)methyl]oxy}benzonitrile;
2-[[2-chloro-4-(trifluoromethyl)phenoxy](phenyl)methyl]morpholine;
2-[(2,5-dichlorophenoxy)(phenyl)methyl]morpholine;
2-[(3-chlorophenoxy)(phenyl)methyl]morpholine;
2-[(2-chloro-3,5-difluorophenoxy)(phenyl)methyl]morpholine;
2-[(4-chloro-2-methoxyphenoxy)(4-fluorophenyl)methyl]morpholine; and
2-[(4-chloro-2-methoxyphenoxy)(3-fluorophenyl)methyl]morpholine.

Additional compounds within the scope of the invention include:
2-[(2,3-dichlorophenoxy)(phenyl)methyl]morpholine;
2-[(2,4-dichlorophenoxy)(phenyl)methyl]morpholine;
2-[(2,3-dichlorophenoxy)(pyridin-2-yl)methyl]morpholine;
2-[(2,3-dichlorophenoxy)(phenyl)methyl]morpholine;
2-{phenyl[2-(trifluoromethoxy)phenoxy]methyl}morpholine;
2-[[2-(difluoromethoxy)phenoxy](phenyl)methyl]morpholine;
2-[(4-chloro-2-methoxyphenoxy)(phenyl)methyl]morpholine;
2-[(3-chloro-2-ethoxyphenoxy)(pyridin-2-yl)methyl]morpholine;
2-[(2,4-dichlorophenoxy)(pyridin-2-yl)methyl]morpholine;
2-[(3-chloro-2-ethoxyphenoxy)(pyridin-2-yl)methyl]morpholine;
2-[(2,3-difluorophenoxy)(4-fluorophenyl)methyl]morpholine;
2-[[4-chloro-2-(methoxymethyl)phenoxy](phenyl)methyl]morpholine;
2-[phenyl(2,3,4-trifluorophenoxy)methyl]morpholine;
2-[(5-fluoro-2-methoxyphenoxy)(phenyl)methyl]morpholine;
2-[(2-methoxy-4-methylphenoxy)(phenyl)methyl]morpholine;
2-[(3-chloro-4-fluorophenoxy)(phenyl)methyl]morpholine;
2-[phenyl(2,3,5-trifluorophenoxy)methyl]morpholine;
2-[(4-chloro-2-methoxyphenoxy)(2-fluorophenyl)methyl]morpholine;
5-{[morpholin-2-yl(phenyl)methyl]oxy}isoquinoline;
2-[(4-chloro-3-methoxyphenoxy)(phenyl)methyl]morpholine;
6-{[morpholin-2-yl(phenyl)methyl]oxy}quinoline;
2-[(2,3-difluorophenoxy)(3-fluorophenyl)methyl]morpholine;
2-[(4-fluoro-2-methoxyphenoxy)(3-fluorophenyl)methyl]morpholine;
7-{[morpholin-2-yl(phenyl)methyl]oxy}quinoline;
7-{[morpholin-2-yl(phenyl)methyl]oxy}isoquinoline;
2-[(4-fluoro-2-methoxyphenoxy)(4-fluorophenyl)methyl]morpholine;
2-[(4-chloro-3-methylphenoxy)(phenyl)methyl]morpholine;
2-[(2,4-dichlorophenoxy)(3-fluorophenyl)methyl]morpholine;
2-[(2-chloro-4-fluorophenoxy)(3-fluorophenyl)methyl]morpholine;
2-[(2,4-difluorophenoxy)(3-fluorophenyl)methyl]morpholine;
2-[(4-chloro-2-methoxyphenoxy)(2-fluorophenyl)methyl]morpholine;
2-[(2,5-difluorophenoxy)(phenyl)methyl]morpholine;
2-[(3-chloro-2-methylphenoxy)(phenyl)methyl]morpholine;
2-[(2-chloro-5-fluorophenoxy)(phenyl)methyl]morpholine;
2-[(5-fluoro-2-methylphenoxy)(phenyl)methyl]morpholine;
2-[(5-chloro-2-methylphenoxy)(phenyl)methyl]morpholine;
2-[(2-chloro-3-fluorophenoxy)(phenyl)methyl]morpholine;
2-[(3-fluoro-2-methoxyphenoxy)(phenyl)methyl]morpholine; and
2-[[2-(difluoromethoxy)-4-fluorophenoxy](phenyl)methyl]morpholine.

In a sixth aspect, the present invention provides for a compound of formula Ib:

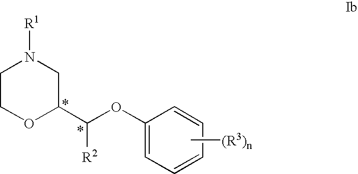

or a pharmaceutically acceptable salt thereof; wherein:
both of the carbons identified with a “*” are of the S conformation;
R¹ is H or C₁₋₆-alkyl;
R² is phenyl or pyridinyl that is optionally substituted by one to three substituents independently selected from C₁₋₆-alkyl, C₁₋₆-alkoxy, OH, halo, CF₃, OCF₃, OCHF₂, or CN;
n is an integer from one to five; and
R³ is independently selected from C₁₋₆alkyl, C₁₋₆alkoxy, OH, halo, CF₃, OCF₃, OCHF₂, or CN;
provided that the compound is not 2-[(2-ethoxyphenoxy)(phenyl)methyl]morpholine.

In certain embodiments of a compound of formula Ib, R² is phenyl that is optionally substituted by one to three substituents independently selected from fluoro, chloro, methyl, or methoxy, R³ is methoxy, chloro, bromo, fluoro, methyl, CF₃, n-propyl, or CN, and R¹ is H. In other embodiments of a compound of formula Ib, n is an integer from one to three, R² is phenyl that is optionally substituted by one to three substituents independently selected from fluoro, chloro, methyl, or methoxy; R³ is methoxy, chloro, bromo, fluoro, methyl, CF₃, n-propyl, or CN; and R¹ is H. In still other embodiments of a compound of formula Ib, said compound is selected from the group consisting of:
(2S)-2-[(1S)-(4-chloro-2-methoxyphenoxy) (phenyl)methyl]morpholine;
(2S)-2-[(1S)-(2,3-Difluorophenoxy)(3-fluorophenyl)methyl]morpholine;
(2S)-2-[(1S)-(3-Chloro-2-fluorophenoxy)phenyl methyl]morpholine;
(2S)-2-[(1S)-(3-Fluorophenyl)-o-tolyloxy-methyl]morpholine;
(2S)-2-[(1S)-(2-Chloro-4-fluorophenoxy)-(3-methoxyphenyl)methyl]morpholine;
(2S)-2-[(1S)-(3-Fluorophenyl)(2-methoxy-4-methylphenoxy)-methyl]morpholine;
(2S)-2-[(1S)-(4-chloro-2-methoxyphenoxy)(pyridin-2-yl)methyl]morpholine;
(2S)-2-[(1S)-(2-Chloro-4-fluorophenoxy)-(3-fluorophenyl)methyl]morpholine; and
(2S)-2-[(1S)-(4-Fluoro-2-methoxyphenoxy)(3-fluorophenyl)methyl]morpholine.

In one embodiment of a compound of formula Ib is (2S)-2-[(1S)-(4-chloro-2-methoxyphenoxy) (phenyl)methyl]morpholine, or a pharmaceutically acceptable salt thereof. Another compound of formula Ib is (2S)-2-[(1S)-(4-chloro-2-methoxyphenoxy) (phenyl)methyl]morpholine. The (2S)-2-[(1S)-(4-chloro-2-methoxyphenoxy) (phenyl)methyl]morpholine may be a besylate salt—(2S)-2-[(1S)-(4-chloro-2-methoxyphenoxy) (phenyl)methyl]morpholine besylate. (2S)-2-[(1S)-(4-chloro-2-methoxyphenoxy) (phenyl)methyl]morpholine besylate may exist in a crystalline form.

In certain embodiments, crystalline (2S)-2-[(1S)-(4-chloro-2-methoxyphenoxy) (phenyl)methyl] morpholine besylate has a X-ray powder diffraction spectrum comprising the following 2-theta values ±0.1 measured using CuKα radiation: 16.6, 18.9, and 22.4. In certain embodiments, crystalline (2S)-2-[(1S)-(4-chloro-2-methoxyphenoxy) (phenyl)methyl]morpholine besylate has a X-ray powder diffraction spectrum comprising the following 2-theta values ±0.1 measured using CuKα radiation: 16.6, 18.9, 19.4, 22.4 and 22.9.

In certain embodiments, crystalline (2S)-2-[(1S)-(4-chloro-2-methoxyphenoxy) (phenyl)methyl] morpholine hydrochloride has a X-ray powder diffraction spectrum comprising the following 2-theta values ±0.1 measured using CuKα radiation: 20.1, 20.9, 23.5, 24.2, and 24.7.

In certain embodiments, crystalline (2S)-2-[(1S)-(4-chloro-2-methoxyphenoxy) (phenyl)methyl] morpholine camsylate has a X-ray powder diffraction spectrum comprising the following 2-theta values ±0.1 measured using CuKα radiation: 12.1, 15.1, 16.4, 18.1, and 25.7.

In certain embodiments, crystalline (2S)-2-[(1S)-(4-chloro-2-methoxyphenoxy) (phenyl)methyl] morpholine citrate has a X-ray powder diffraction spectrum comprising the following 2-theta values ±0.1 measured using CuKα radiation: 11.7, 19.7, 22.7, and 24.5.

In certain embodiments, crystalline (2S)-2-[(1S)-(4-chloro-2-methoxyphenoxy) (phenyl)methyl] morpholine tartrate has a X-ray powder diffraction spectrum comprising the following 2-theta values ±0.1 measured using CuKα radiation: 13.1, 20.0, 21.9, and 22.9.

In certain embodiments, crystalline (2S)-2-[(1S)-(4-chloro-2-methoxyphenoxy) (phenyl)methyl] morpholine fumarate has a X-ray powder diffraction spectrum comprising the following 2-theta values ±0.1 measured using CuKα radiation: 18.4, 20.0, 23.9, and 27.4.

In certain embodiments, crystalline (2S)-2-[(1S)-(4-chloro-2-methoxyphenoxy) (phenyl)methyl] morpholine hydrobromide has a X-ray powder diffraction spectrum comprising the following 2-theta values ±0.1 measured using CuKα radiation: 20.5, 21.1, 23.1, 23.8, and 25.4.

In certain embodiments, crystalline (2S)-2-[(1S)-(4-chloro-2-methoxyphenoxy) (phenyl)methyl] morpholine edisylate has a X-ray powder diffraction spectrum comprising the following 2-theta values ±0.1 measured using CuKα radiation: 3.4, 4.7, 5.2, 18.5, and 19.9.

In certain embodiments, crystalline (2S)-2-[(1S)-(4-chloro-2-methoxyphenoxy) (phenyl)methyl] morpholine succinate has a X-ray powder diffraction spectrum comprising the following 2-theta values ±0.1 measured using CuKα radiation: 11.8, 18.2, 20.0, and 23.5.

Compounds of formula Ib may be present in a composition comprising: a therapeutically effective amount of a compound according of formula Ib, or a pharmaceutically acceptable salt thereof, and a pharmaceutically acceptable carrier.

Compounds of formula Ib may be used in the manufacture of a medicament for the treatment of a disorder selected from the group consisting of: ADHD, genuine stress incontinence, stress urinary incontinence, depression, generalised anxiety disorder, fibromyalgia, and pain. In a particular embodiments, the compound is (2S)-2-[(1S)-(4-chloro-2-methoxyphenoxy) (phenyl)methyl]morpholine, or a pharmaceutically acceptable salt thereof.

According to a seventh aspect of the invention, there is provided a compound of Formula I, Ia, or Ib as defined above for use as a pharmaceutical.

According to an eighth aspect of the invention, there is provided a compound of Formula I, a, or Ib for use in the treatment of a disorder in which the regulation of monoamine transporter function in mammals is implicated.

According to a ninth aspect of the invention, there is provided a use of a compound of Formula I, Ia, or Ib as defined above in the manufacture of a medicament for the treatment of a disorder in which the regulation of monoamine transporter function in mammals is implicated.

An embodiment of the ninth aspect of the invention includes the treatment of a disorder in which the regulation of serotonin or noradrenaline in mammals is implicated.

A further embodiment includes the treatment of a disorder in which the regulation of serotonin and noradrenaline is implicated.

A still further embodiment includes the manufacture of a medicament for the treatment of urinary disorders, depression, pain, premature ejaculation, ADHD or fibromyalgia in mammals, in particular, the treatment of urinary incontinence, such as GSI or SUI, in mammals, and the treatment of fibromyalgia.

According to a tenth aspect of the invention, there is provided a method of treating a disorder in which the regulation of monoamine transporter function is implicated which comprises administering a therapeutically effective amount of a compound of Formula I, Ia, or Ib as defined above to a patient in need of such treatment.

An embodiment of the tenth aspect of the invention includes a method of treating a disorder in which the regulation of serotonin or noradrenaline is implicated.

A further embodiment includes a method of treating a disorder wherein the regulation of serotonin and noradrenaline is implicated.

A still further embodiment includes a method of treating urinary disorders, depression, pain, premature ejaculation, ADHD or fibromyalgia, which comprises administering a therapeutically effective amount of a compound of Formula I, Ia, or Ib as defined above to a patient in need of such treatment, in particular urinary incontinence, such as GSI or SUI, and fibromyalgia.

In an eleventh aspect, the present invention provides for methods of treating a disorder selected from the group consisting of: ADHD, genuine stress incontinence, stress urinary incontinence, depression, generalised anxiety disorder, fibromyalgia, and pain, comprising administering to a mammal in need thereof, a therapeutically effective amount of a compound of Formula I, Ia, or Ib, and a pharmaceutically acceptable carrier. In certain embodiments, the compound is (2S)-2-[(1S)-(4-chloro-2-methoxyphenoxy) (phenyl)methyl]morpholine, or a pharmaceutically acceptable salt thereof. In other embodiments, the disorder is fibromyalgia and the compound of formula I is (2S)-2-[(1S)-(4-chloro-2-methoxyphenoxy) (phenyl)methyl]morpholine, or a pharmaceutically acceptable salt thereof.

According to a twelvth aspect of the invention, there is provided a process for the preparation of a compound of Formula Ia as defined above, the process including either (i) reacting a compound of formula VIII:

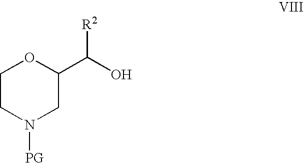

wherein PG is a suitable protecting group, with a phenol compound of formula:

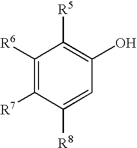

under suitable conditions, followed by deprotection as necessary; or
(ii) cyclising a compound of formula XVIIa:

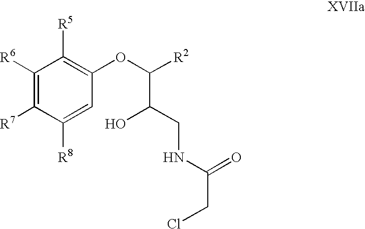

to provide a compound of formula XVIIIa:

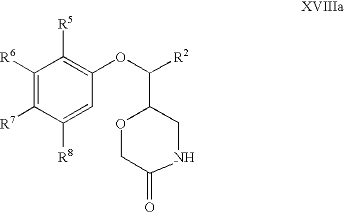

followed by removal of the carbonyl oxygen (═O) from the morpholinone group.

The substituent R⁴ is defined above as a phenyl group fused to a 5- or 6-membered carbocyclic group, or a phenyl group fused to a 5- or 6-membered heterocyclic group containing at least one N, O or S heteroatom. However, in connection with any of the embodiments mentioned above, R⁴ may be a phenyl group fused to a 6-membered carbocyclic group, or a phenyl group fused to a 5- or 6-membered heterocyclic group containing at least one N or O heteroatom.

In the above definitions of the compounds of Formula I or Formula Ia, the term “aryl” means phenyl, naphthyl, anthracyl or phenanthryl. However, in connection with any of the embodiments mentioned above, “aryl” may be phenyl or naphthyl.

The term “het” is defined above as an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom. However, in connection with any of the embodiments mentioned above, het may be an aromatic or non-aromatic 5- or 6-membered heterocycle which contains at least one N or O heteroatom, optionally fused to a 5- or 6-membered carbocyclic group or a second 5- or 6-membered heterocycle which contains at least one N or O heteroatom; or an aromatic or non-aromatic 5- or 6-membered heterocycle which contains at least one N heteroatom, optionally fused to a 5- or 6-membered carbocyclic group or a second 5- or 6-membered heterocycle which contains at least one N heteroatom. In the preceding definitions, the second heterocycle, to which the first heterocycle may be fused, may be either aromatic or non-aromatic.

In the compounds of Formula I or Ia, R² may be optionally substituted by at least one substituent independently selected from C₁₋₆alkyl, C₁₋₆alkoxy, OH, halo, CF₃, CN, when R² contains a cycloalkyl, aryl or het group.

Alternatively, R² may be aryl, a 5- or 6-membered aromatic or non-aromatic heterocycle containing at least one N or O heteroatom or —(CH₂)_{z}aryl, wherein z is an integer from 1 to 3 and aryl is as defined above.

According to a further aspect of the invention, there is provided one or more metabolites of the compounds of Formula I, Ia or Ib when formed in vivo.

By pharmaceutically and/or veterinarily acceptable derivative it is meant any pharmaceutically or veterinarily acceptable salt or solvate of the compounds of Formula I, Ia or Ib.

For pharmaceutical or veterinary use, the salts referred to above will be the pharmaceutically or veterinarily acceptable salts, but other salts may find use, for example in the preparation of compounds of Formula I, Ia, or Ib and the pharmaceutically or veterinarily acceptable salts thereof.

The aforementioned pharmaceutically or veterinarily acceptable salts include the acid addition and base salts thereof.

Suitable acid addition salts are formed from acids which form non-toxic salts. Examples include the acetate, aspartate, benzoate, besylate, bicarbonate/carbonate, bisulphate/sulphate, camsylate, citrate, edisylate, hemiedisylate, esylate, fumarate, gluceptate, gluconate, glucuronate, hibenzate, hydrochloride/chloride, hydrobromide/bromide, hydroiodide/iodide, isethionate, lactate, malate, maleate, malonate, mesylate, methylsulphate, 2-napsylate, nicotinate, nitrate, orotate, pamoate, phosphate/hydrogen phosphate/dihydrogen phosphate, saccharate, stearate, succinate, tartrate and tosylate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of Formula I, Ia, or Ib may be readily prepared by mixing together solutions of the compound and the desired acid or base, as appropriate. The salt may precipitate from solution and be collected by filtration or may be recovered by evaporation of the solvent. The degree of ionisation in the salt may vary from completely ionised to almost non-ionised.

Pharmaceutically acceptable solvates in accordance with the invention include hydrates and solvates of the compounds of Formula I, Ia, or Ib.

Also within the scope of the invention are complexes such as clathrates, drug-host inclusion complexes wherein, in contrast to the aforementioned solvates, the drug and host are present in stoichiometric or non-stoichiometric amounts. Also included in this invention are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of Formula I, Ia, or Ib may be modified to provide pharmaceutically or veterinarily acceptable derivatives thereof at any of the functional groups in the compounds. Examples of such derivatives are described in: Drugs of Today, Volume 19, Number 9, 1983, pp 499-538; Topics in Chemistry, Chapter 31, pp 306-316; and in “Design of Prodrugs” by H. Bundgaard, Elsevier, 1985, Chapter 1 (the disclosures in which documents are incorporated herein by reference) and include: esters, carbonate esters, hemi-esters, phosphate esters, nitro esters, sulfate esters, sulfoxides, amides, sulphonamides, carbamates, azo-compounds, phosphamides, glycosides, ethers, acetals and ketals.

It will be further appreciated by those skilled in the art, that certain moieties, known in the art as “pro-moieties”, for example as described by H. Bundgaard in “Design of Prodrugs” (ibid) may be placed on appropriate functionalities when such functionalities are present within compounds of the invention.

The compounds of Formula I, Ia or Ib may contain one or more chiral centers. Such compounds exist in a number of stereoisomeric forms (e.g. in the form of a pair of optical isomers, or enantiomers). Unless otherwise specified, it is to be understood that the present invention encompasses all isomers of the compounds of the invention, including all geometric, tautomeric and optical forms, and mixtures thereof (e.g. tautomeric or racemic mixtures).

The compounds of Formula I, Ia or Ib may exist in one or more tautomeric forms. All tautomers and mixtures thereof are included in the scope of the present invention. For example, a claim to 2-hydroxypyridinyl would also cover its tautomeric form α-pyridonyl.

It is to be understood that the present invention includes radiolabelled compounds of Formula I, Ia or Ib.

The compounds of Formula I, Ia or Ib and their pharmaceutically and veterinarily acceptable derivatives thereof may also be able to exist in more than one crystal form, a characteristic known as polymorphism. All such polymorphic forms (“polymorphs”) are encompassed within the scope of the invention. Polymorphism generally can occur as a response to changes in temperature or pressure or both, and can also result from variations in the crystallisation process. Polymorphs can be distinguished by various physical characteristics, and typically the x-ray diffraction patterns, solubility behaviour, and melting point of the compound are used to distinguish polymorphs.

Unless otherwise indicated, any alkyl group may be straight or branched and is of 1 to 8 carbon atoms, such as 1 to 6 carbon atoms or 1 to 4 carbon atoms, for example a methyl, ethyl, n-propyl, i-propyl, n-butyl, i-butyl, s-butyl or t-butyl group. Where the alkyl group contains more than one carbon atom, it may be unsaturated. Thus, the term C₁₋₆ alkyl includes C₂₋₆ alkenyl and C₂₋₆ alkynyl. Similarly, the term C₁₋₈ alkyl includes C₂₋₈ alkenyl and C₂₋₈ alkynyl, and the term C₁₋₄ alkyl includes C₂₋₄ alkenyl and C₂₋₄ alkynyl.

The term halogen is used to represent fluorine, chlorine, bromine or iodine.

Unless otherwise indicated, the term het includes any aromatic, saturated or unsaturated 4-, 5- or 6-membered heterocycle which contains up to 4 heteroatoms selected from N, O and S. Examples of such heterocyclic groups included furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazapinyl, thiazepinyl, diazepinyl and thiazolinyl. In addition, the term heterocycle includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazdinyl, benzothiazolyl, phthalimido, benzodiazepinyl, indolyl and isoindolyl. The terms het, heterocyclyl and heterocyclic should be similarly construed.

For the avoidance of doubt, unless otherwise indicated, the term “substituted” means substituted by one or more defined groups. In the case where groups may be selected from a number of alternative groups, the selected groups may be the same or different. Further, the term “independently” means that where more than one substituent is selected from a number of possible substituents, those substituents may be the same or different.

Hereinafter, the compounds of Formula I, Ia or Ib and their pharmaceutically and veterinarily acceptable derivatives, the radiolabelled analogues of the foregoing, the isomers of the foregoing, and the polymorphs of the foregoing, may be referred to as “the compounds of the invention”.

In one embodiment of the invention, the compounds of the invention are the pharmaceutically and veterinarily acceptable derivatives of compounds of Formula I, Ia, or Ib, such as the pharmaceutically or veterinarily acceptable salts or solvates of compounds of Formula I, Ia, or Ib (e.g. pharmaceutically or veterinarily acceptable salts of compounds of Formula I, Ia, or Ib).

In a still further embodiment of the invention, there is provided a compound of Formula I, Ia, or Ib which is an inhibitor of serotonin and/or noradrenaline monoamine re-uptake, having SRI or NRI Ki values of 200 nM or less. In a further embodiment, the compound has SRI and/or NRI Ki values of 100 nM or less. In a yet further embodiment, the compound has SRI or NRI Ki values of 50 nM or less. In a still further embodiment, the compound has SRI and NRI Ki values of 50 nM or less. In a still yet further embodiment, the compound has SRI and NRI Ki values of 25 nM or less.

Without wishing to be bound by theory, it is believed that the utility of the compounds of the invention in the aforementioned indications is a result of their combined SRI and NRI activities.

### BRIEF DESCRIPTIONS OF THE DRAWINGS

FIGS. 1-9 are powder x-ray diffraction (PXRD) spectra of: (2S)-2-[(S)-(4-chloro-2-methoxyphenoxy)(phenyl)methyl]morpholine besylate (FIG. 1); (2S)-2-[(S)-(4-chloro-2-methoxyphenoxy)(phenyl)methyl]morpholine hydrochloride (FIG. 2); (2S)-2-[(S)-(4-chloro-2-methoxyphenoxy)(phenyl)methyl]morpholine camsylate (FIG. 3); (2S)-2-[(S)-(4-chloro-2-methoxyphenoxy)(phenyl)methyl]morpholine citrate (FIG. 4); (2S)-2-[(S)-(4-chloro-2-methoxyphenoxy)(phenyl)methyl]morpholine L-tartrate (FIG. 5); (2S)-2-[(S)-(4-chloro-2-methoxyphenoxy)(phenyl)methyl]morpholine fumarate (FIG. 6); (2S)-2-[(S)-(4-chloro-2-methoxyphenoxy)(phenyl)methyl]morpholine hydrobromide (FIG. 7); (2S)-2-[(S)-(4-chloro-2-methoxyphenoxy)(phenyl)methyl]morpholine edisylate (FIG. 8); and (2S)-2-[(S)-(4-chloro-2-methoxyphenoxy)(phenyl)methyl]morpholine succinate (FIG. 9). The X-axis is the 2-theta scale and the y-axis is the linear (Lin) counts.

FIGS. 10-18 are differential scanning calorimetry thermal profiles of: (2S)-2-[(S)-(4-chloro-2-methoxyphenoxy)(phenyl)methyl]morpholine besylate (FIG. 10); (2S)-2-[(S)-(4-chloro-2-methoxyphenoxy)(phenyl)methyl]morpholine hydrochloride (FIG. 11); (2S)-2-[(S)-(4-chloro-2-methoxyphenoxy)(phenyl)methyl]morpholine camsylate (FIG. 12); (2S)-2-[(S)-(4-chloro-2-methoxyphenoxy)(phenyl)methyl]morpholine citrate (FIG. 13); (2S)-2-[(S)-(4-chloro-2-methoxyphenoxy)(phenyl)methyl]morpholine L-tartrate (FIG. 14); (2S)-2-[(S)-(4-chloro-2-methoxyphenoxy)(phenyl)methyl]morpholine fumarate (FIG. 14); (2S)-2-[(S)-(4-chloro-2-methoxyphenoxy)(phenyl)methyl]morpholine hydrobromide (FIG. 15); (2S)-2-[(S)-(4-chloro-2-methoxyphenoxy)(phenyl)methyl]morpholine edisylate (FIG. 16); and (2S)-2-[(S)-(4-chloro-2-methoxyphenoxy)(phenyl)methyl]morpholine succinate (FIG. 17).

FIG. 19 is a calculated powder x-ray diffraction (PXRD) spectrum of: (2S)-2-[(S)-(4-chloro-2-methoxyphenoxy)(phenyl)methyl]morpholine besylate

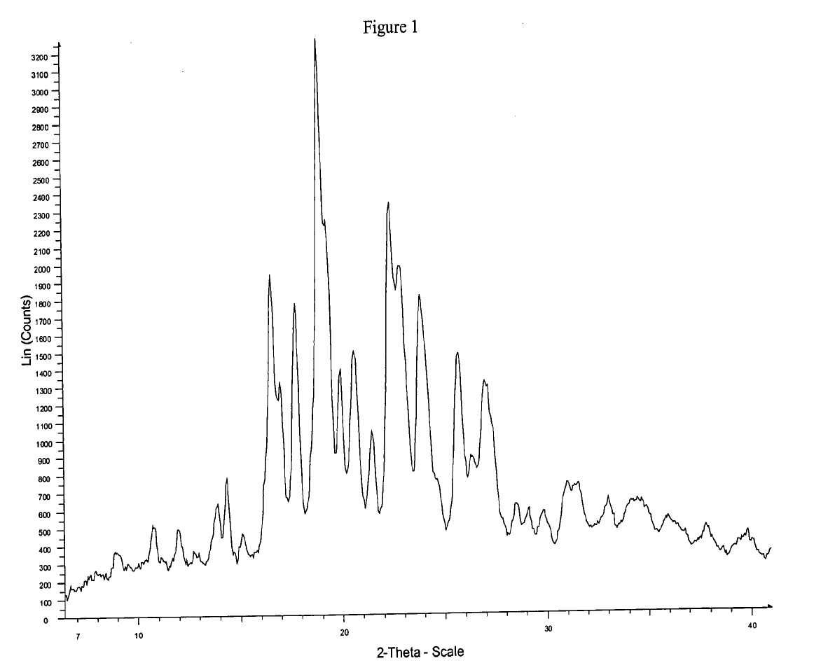

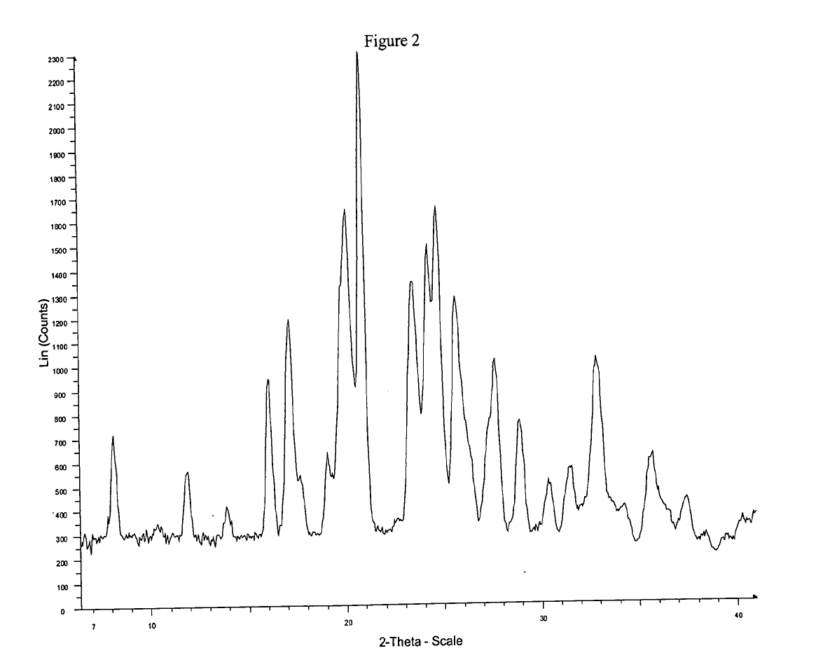

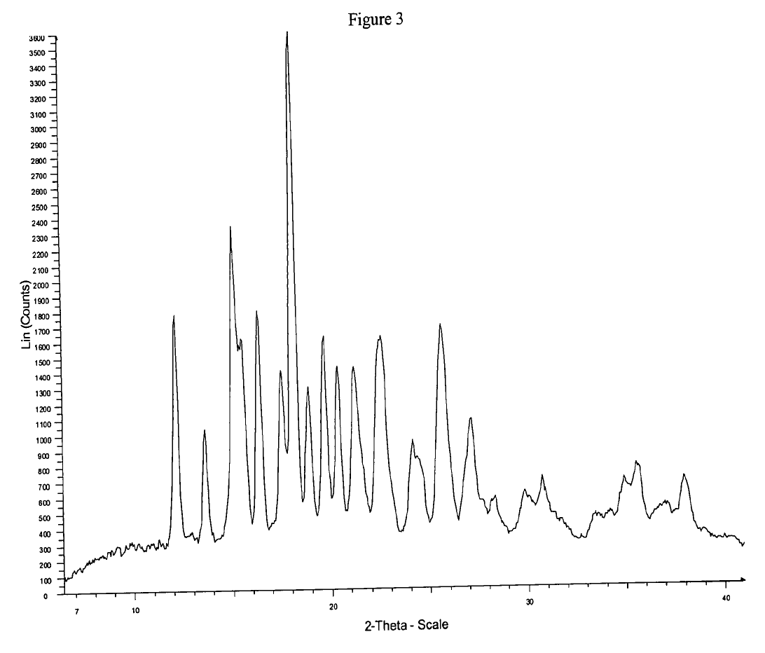

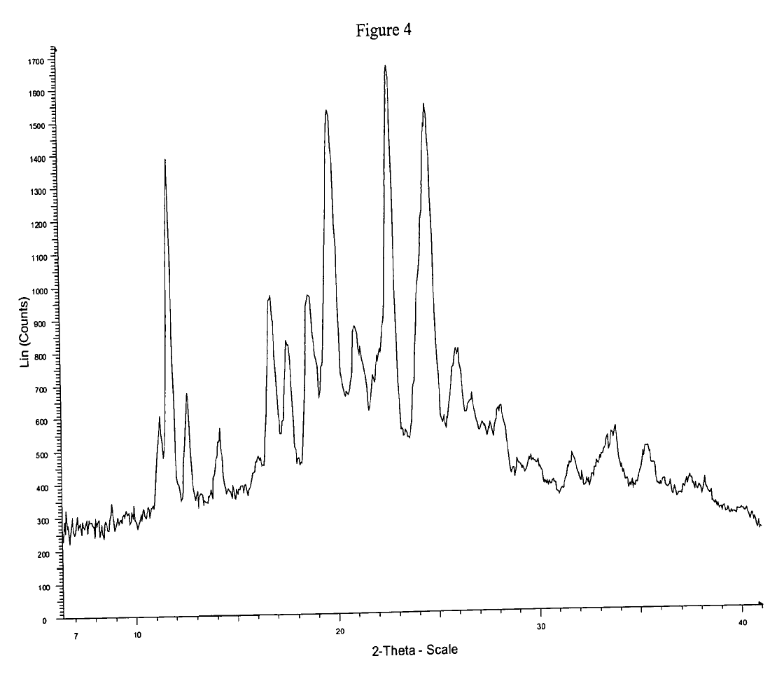

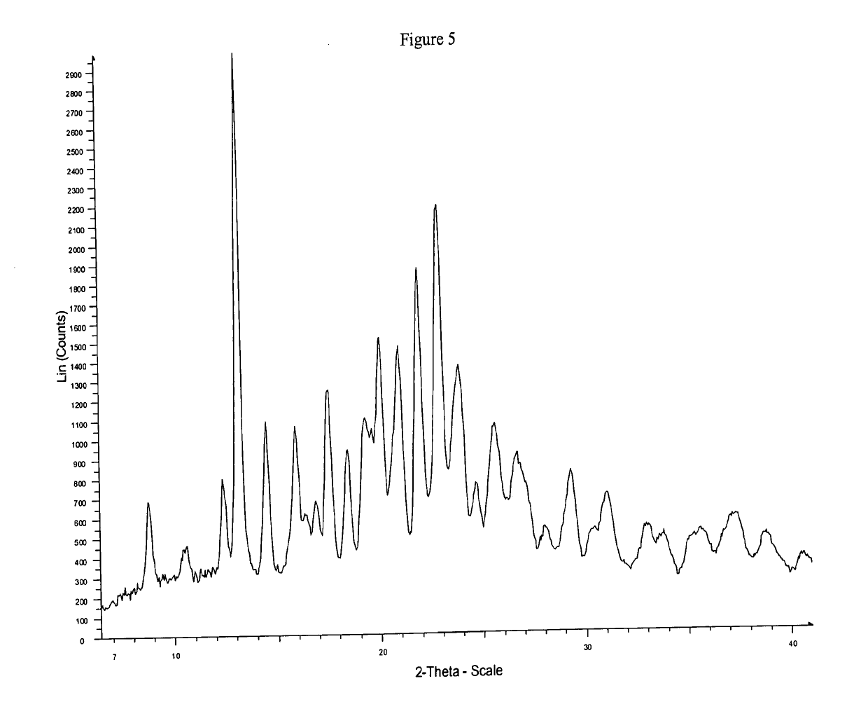

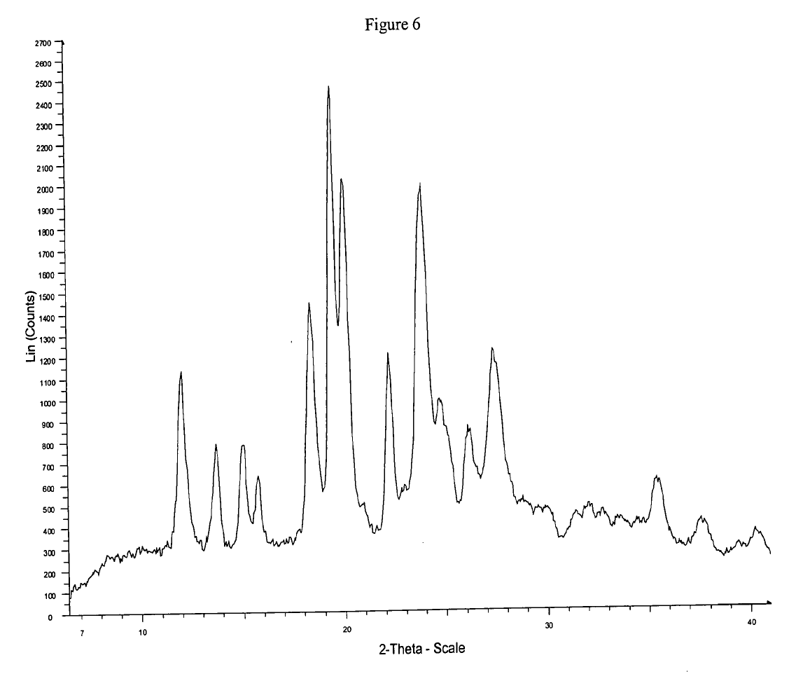

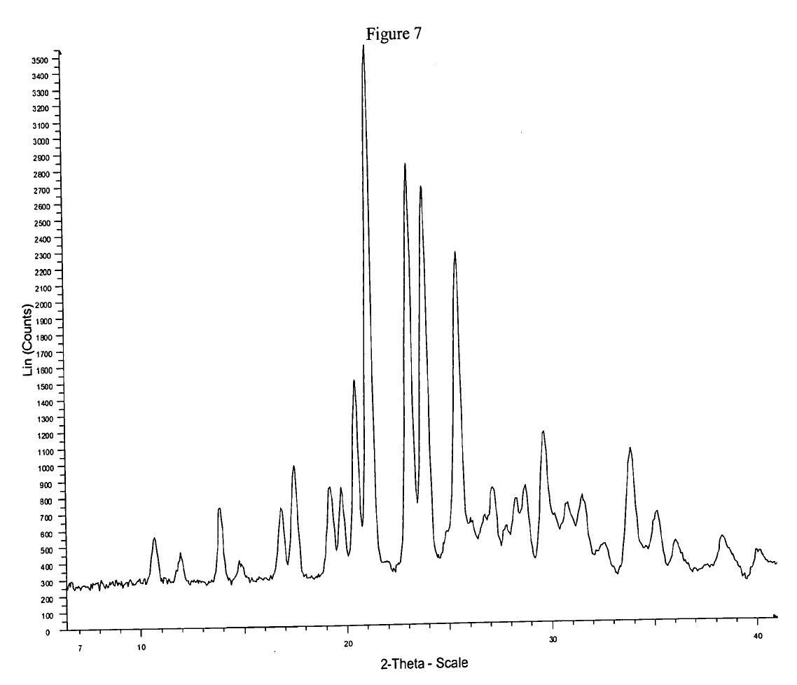

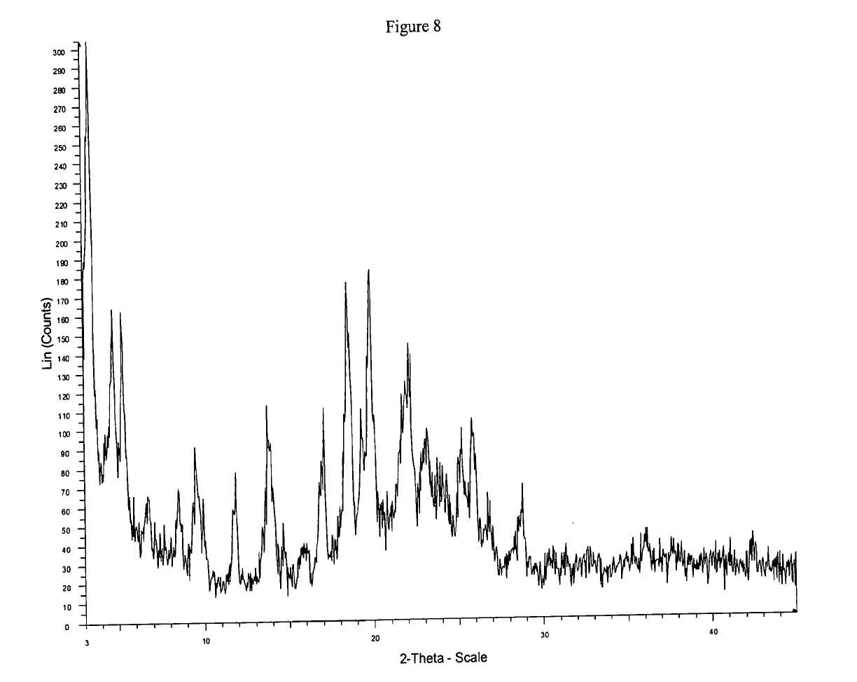

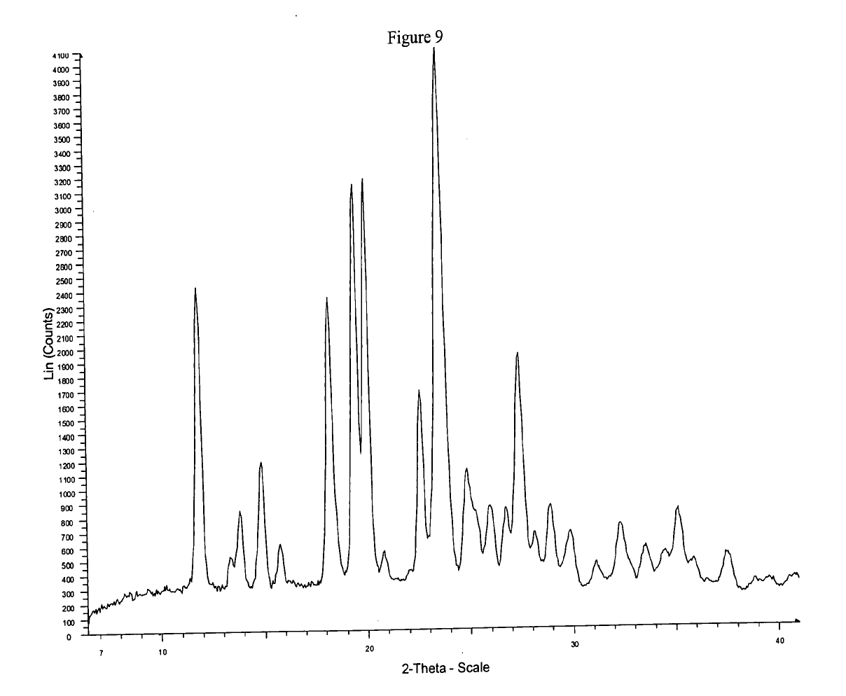

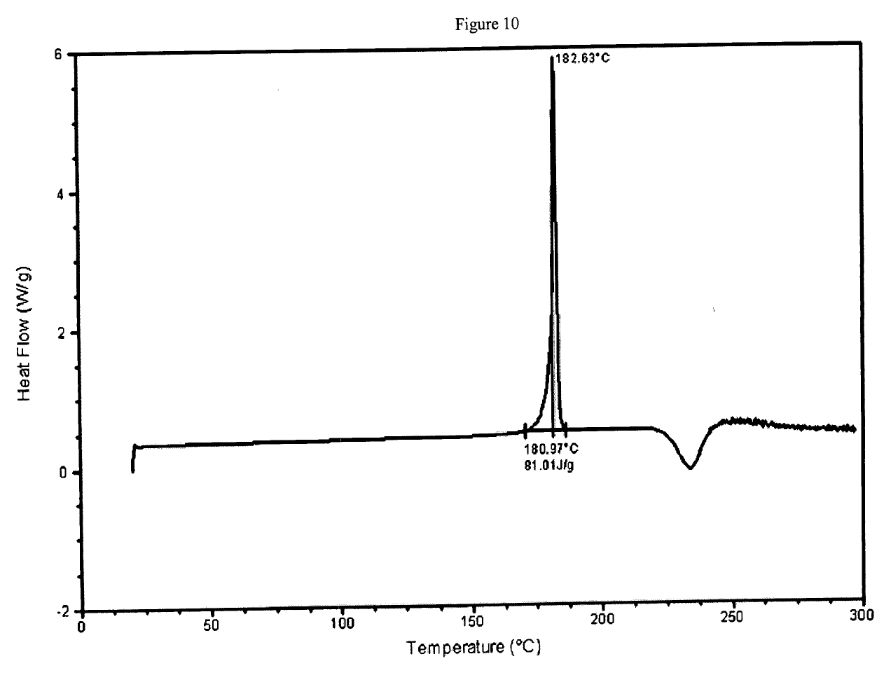

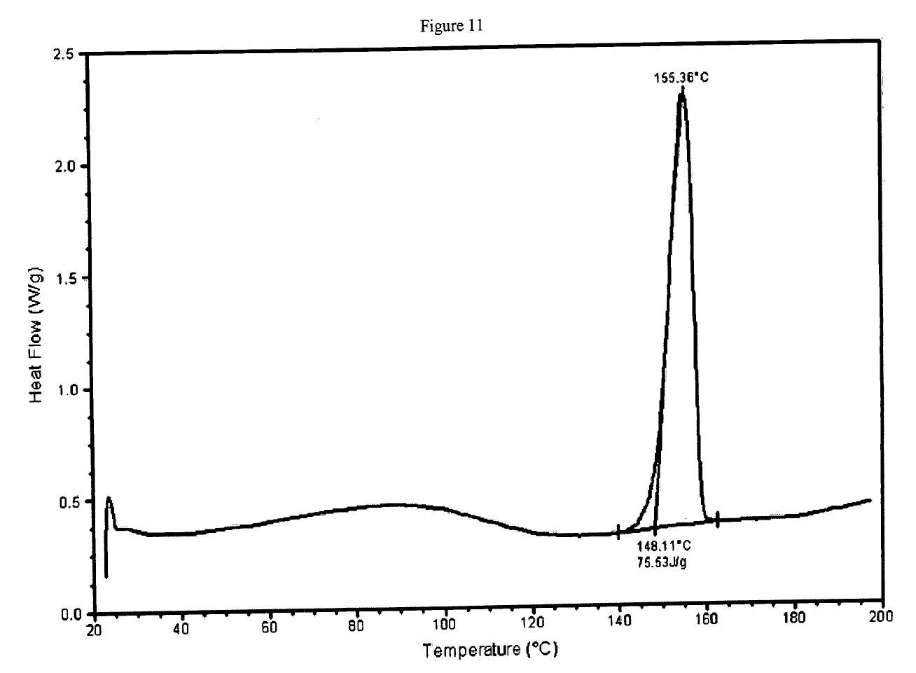

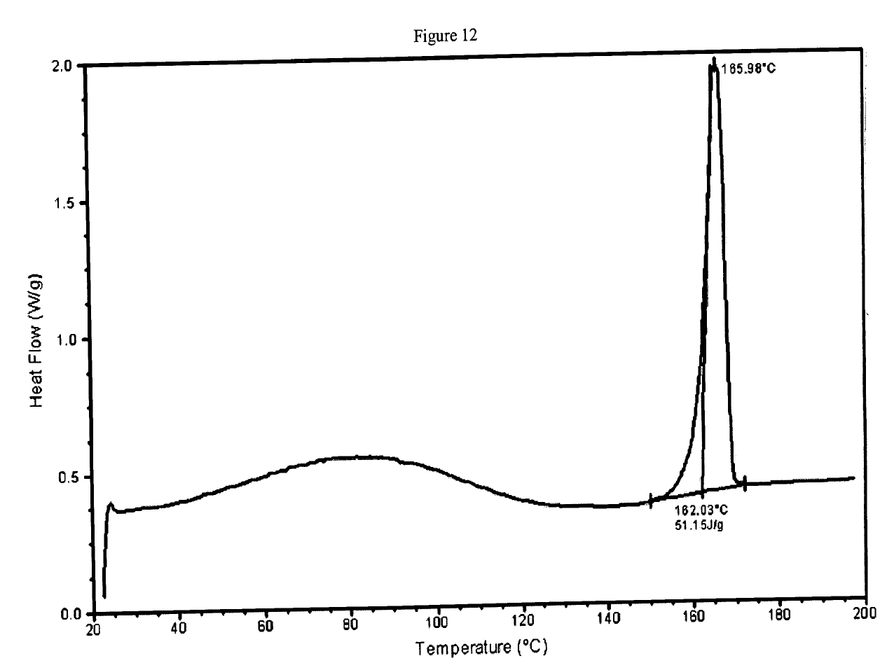

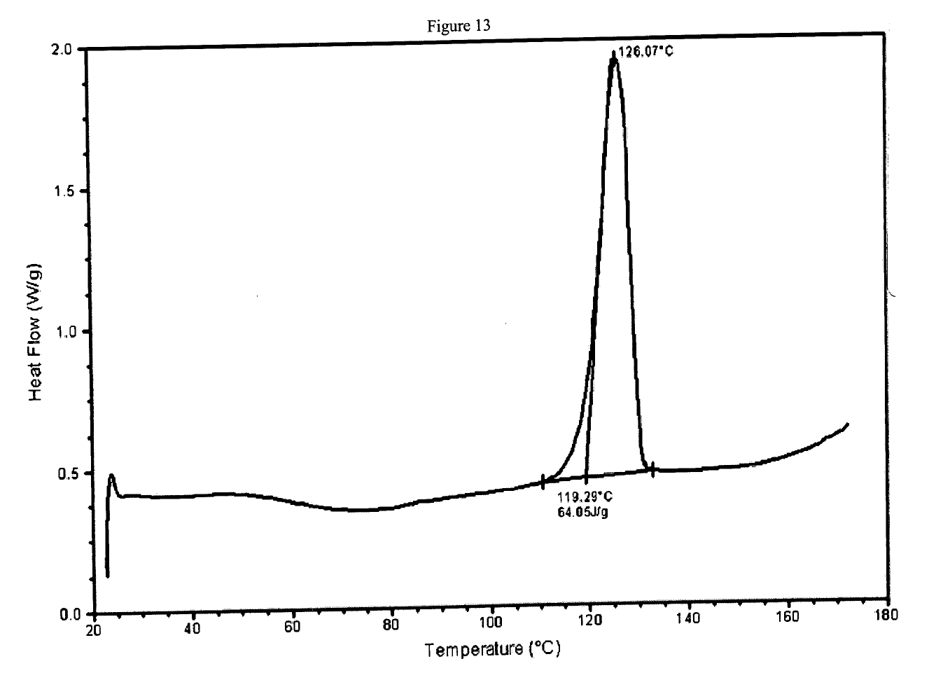

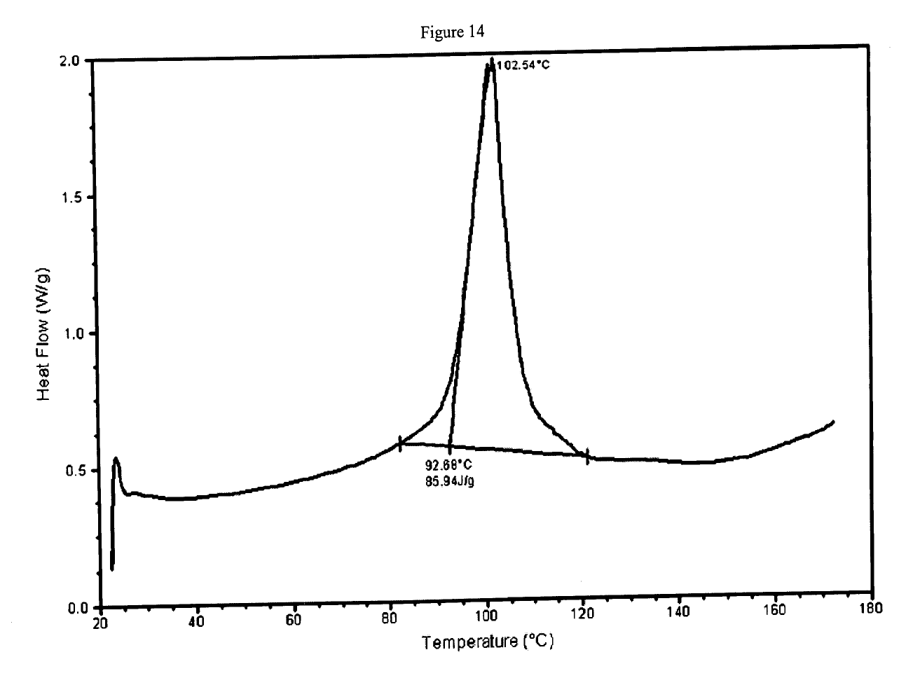

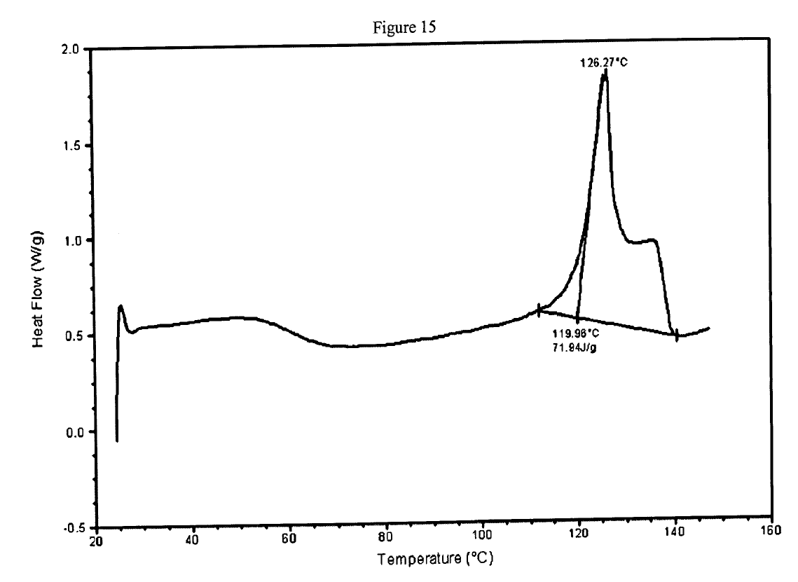

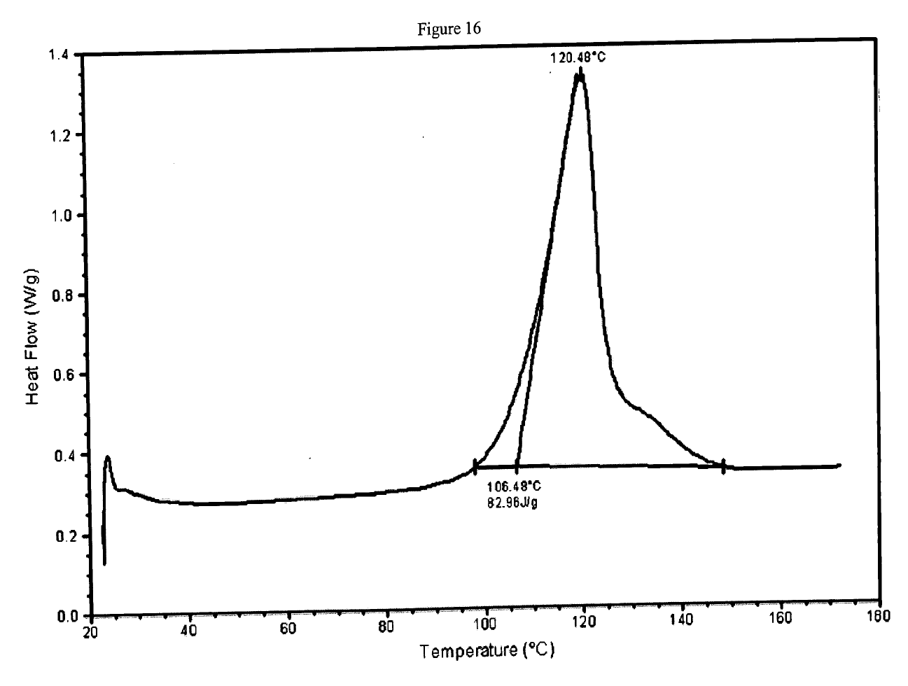

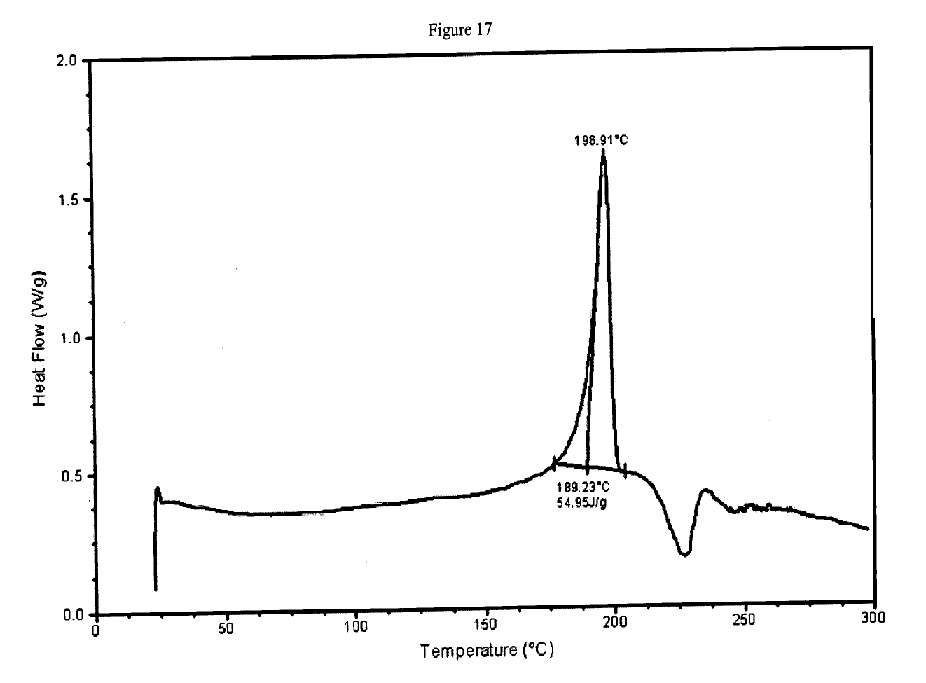

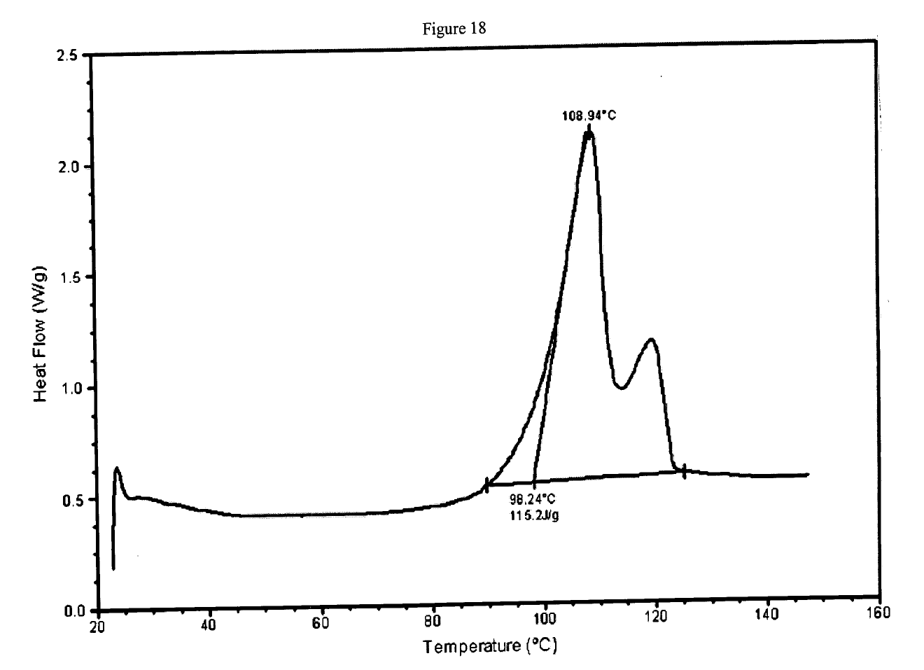

### DETAILED DESCRIPTION

According to Scheme 1, compounds of Formula I:

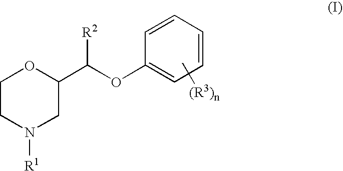

may be prepared in a variety of ways. The routes below illustrate one such way of preparing these compounds; the skilled man will appreciate that other routes may be equally as practicable.

Racemic compounds of general formula (I), where R¹=H and R² and R³ are as described herein, may be prepared according to reaction Scheme 1.

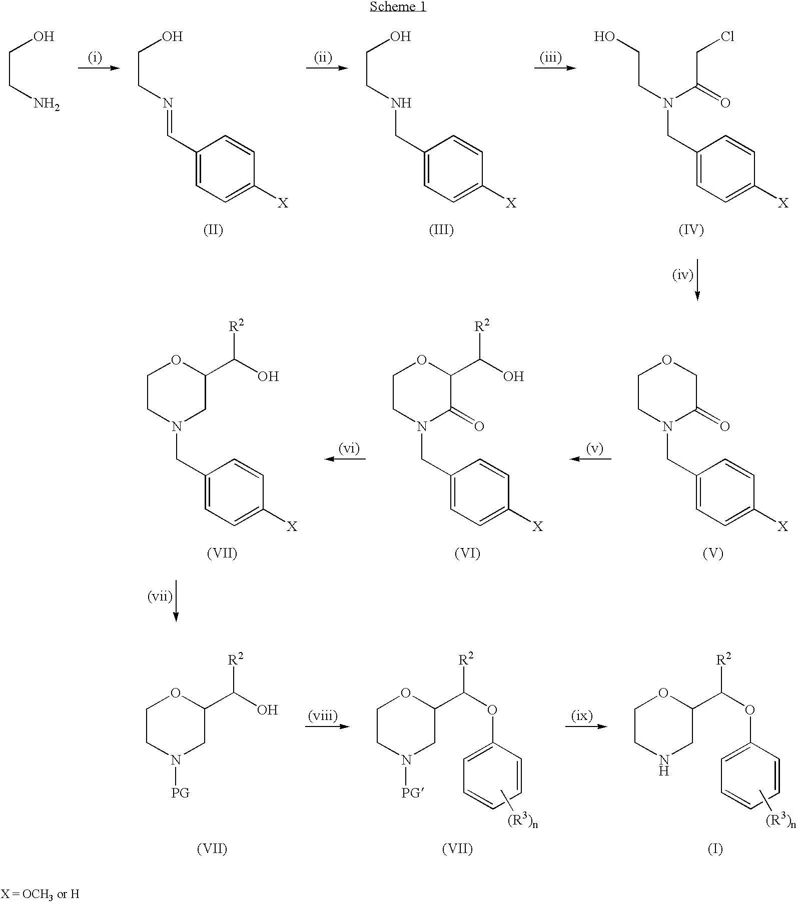

Compounds of general formula (II) can be prepared from ethanolamine by process steps (i)—Reaction with aldehyde ArC(O)H in a suitable solvent such as methanol or ethanol, at ambient temperature for 10-24 hours. Typical conditions consist of 1.0 equivalent of ethanolamine with 1.0 equivalent of aldehyde in methanol at room temperature, for 18 hours.

Compounds of general formula (III) can be prepared from compounds of general formula (II) by process steps (ii)—Reduction with a suitable reducing agent such as sodium cyanoborohydride or sodium triacetoxyborohydride, or alternatively hydrogen gas in the presence of a suitable hydrogenation catalyst such as platinum oxide or Pd/C, in a suitable solvent such as methanol, ethanol or tetrahydrofuran, at ambient temperature for 4-8 hours. Typical conditions consist of 1.0 equivalent of compound (II) in the presence of 30 psi hydrogen gas and platinum oxide (cat), in methanol, at room temperature for 4 hours.

Alternatively, when X═H, compound (III) is commercially available.

Compounds of general formula (IV) can be prepared from compounds of general formula (III) by process steps (iii)—Reaction with chloroacetyl chloride in the presence of a suitable base such as sodium hydroxide or N-methylmorpholine in a suitable biphasic system such as dichloromethane or tetrahydrofuran and water, at ambient temperature for 3-18 hours. Typical conditions comprise of 1.0 equivalent of compound (1II), 1.0-1.3 equivalents of chloroacetyl chloride and 1.0 equivalent of sodium hydroxide in dichloromethane and water, at room temperature for 3 hours.

Compounds of general formula (V) can be prepared from compounds of general formula (IV) by process steps (iv)—Reaction with a suitable base such as potassium hydroxide or caesium carbonate, in a suitable solvent such as ethanol or methanol, at ambient temperature for 4-90 hours. Typical conditions consist of 1.0 equivalent of compound (1V) with 1.0 equivalent of potassium hydroxide in methanol, at room temperature for 6 hours.

Compounds of general formula (VI) can be prepared from compounds of general formula (V) by reaction step (v)—De-protonation with a suitable base, optionally generated in situ, such as lithium diisopropylamide or sodium hexamethyldisilazane and reaction with a suitable aldehyde R²CHO, in presence a suitable solvent such tetrahydrofuran, at low temperature for 1-6 hours. Typical conditions comprise of 1.0 equivalent of compound (V), 1.0-2.0 equivalents of generated lithium diisopropylamide and 1.0-2.0 equivalents of aldehyde R²CHO in tetrahydrofuran, at −78° C. for 3 hours.

Compounds of general formula (VII) can be prepared from compounds of general formula (VI) by reaction step (vi). Reduction with a suitable reducing agent such as borane in tetrahydrofuran, lithium aluminium hydride or Red AI™, in a suitable solvent such as tetrahydrofuran, methanol or diethyl ether, at ambient temperature for 2-48 hours. Typical conditions comprise of 1.0 equivalent of compound (IV) and 4.0 equivalents of borane in tetrahydrofuran, at room temperature for 48 hours.

Compounds of general formula (VII) can be prepared from compounds of general formula (VII) by process step (vii). Aryl group can be optionally substituted with a protecting group PG such as t-BOC or CBz. Aryl group can removed by hydrogenation, in the presence of a suitable hydrogen donor such as 1-methyl-1,4-cyclohexadiene or ammonium formate and a hydrogenation catalyst such as 10% Pd/C, and the ‘free’ morpholine can be treated with a source of protecting group such as di-tert-butyl dicarbonate, in a suitable solvent such as methanol or ethanol, at elevated temperature, for 3-24 hours. Typical conditions comprise of 1.0 equivalent of compound (VII), 3.0-3.5 equivalents of 1-methyl-1,4-cyclohexadiene, 10% Pd/C and 1.0-1.2 equivalents of di-tert-butyl dicarbonate in ethanol, heated under reflux for 2-8 hours.

Compounds of general formula (VIII) can also undergo an inversion in their stereochemistry to the more preferred diastereoisomer (VIIIb) as shown in Scheme 3.

Compounds of general formula (IX) can be prepared from compounds of general formula (VIII) by process step (viii)—A Mitsunobu reaction with a suitable phenol (R³)ₙPh—OH in the presence of a suitable phosphine such as tri-n-butyl phosphine or triphenyl phosphine and a suitable azo compound such as diisopropylazodicarboxylate, di-tert-butyl azodicarboxylate or 1′,1′-azobis(N,N-dimethylformamide), in a solvent such as toluene, tetrahydrofuran or N,N-dimethylformamide, at temperatures between 25-115° C., for 1-48 hours. Typical conditions comprise of 1.0 equivalent of compound (VIII), 1.0-2.0 equivalents of (R³)ₙPh—OH, 1.0-1.5 equivalents of tri-phenylphosphine and 1.0-1.3 equivalents of diisopropylazodicarboxylate in toluene, at 25° C. for 18 hours.

Compounds of general formula (I) can be prepared from compounds of general formula (IX) by process step (ix)—De-protection of compound (IX) may be achieved using standard methodology as described in “Protecting Groups in Organic Synthesis” by T. W. Greene and P. Wutz. When PG=t-BOC, typical conditions comprise of 1.0 equivalent of compound (IX) in the presence of hydrochloric acid (4M in dioxan), in dichloromethane, at room temperature for 18 hours. Alternatively, when PG=benzyl, typical conditions comprise of 1.0 equivalent of compound (IX), 2.0 equivalents of Chloroethyl chloroformate and 1.0 equivalent of Proton Sponge™ in dichloromethane, at room temperature for 18 hours.

Alternatively, homochiral compounds of general formula (I), where R¹=H and R² and R³ are as described herein, may also be prepared according to reaction Scheme 2.

Scheme 2 shows the homochiral route to the (1R,2R) diastereoisomer but a man skilled in the art will appreciate that the (1S,2S) diastereoisomer may also be prepared using a similar route.

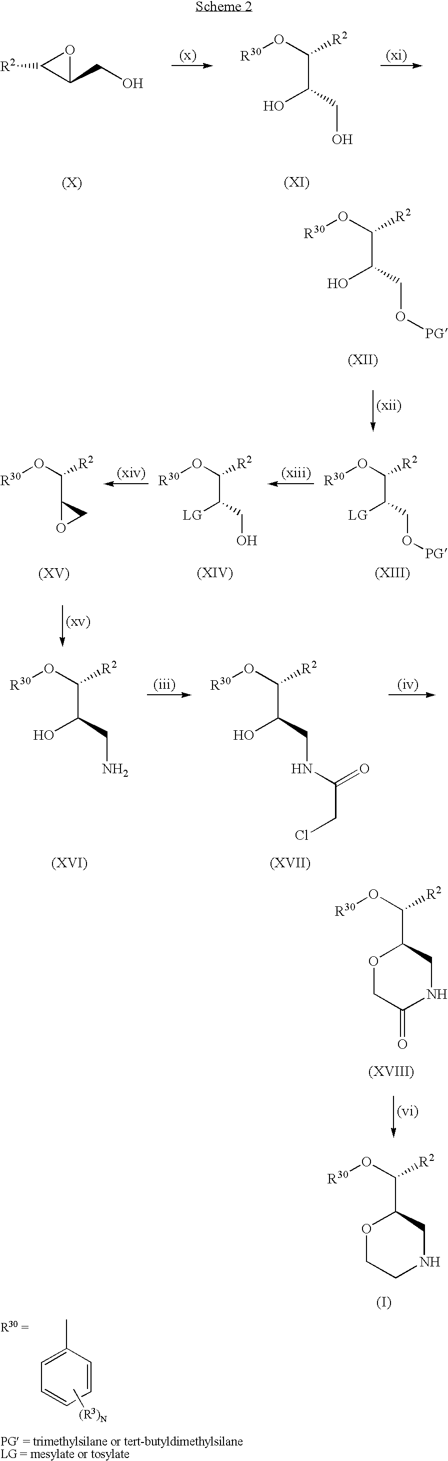

Compounds of general formula (X) are either commercial or can be prepared as described in the literature.

Compounds of general formula (XI) can be prepared from compounds of general formula (X) by process step (x)—Reaction with a suitable phenol ((R³)ₙPh—OH), in the presence of a suitable base such as sodium hydroxide or potassium hydroxide and a suitable phase transfer catalyst such as methyltri-n-butylammonium chloride or tetrabutyl ammonium chloride, in a biphasic solvent system such as dichloromethane and water, at elevated temperature for 1-10 hours. Typical conditions comprise of 1.0 equivalent of compound (X), 2.0 equivalents of phenol (R³)ₙPh-OH, excess sodium hydroxide and methyltri-n-butylammonium chloride (cat), in dichloromethane and water (50:50), heated under reflux for 7 hours.

Compounds of general formula (XII) can be prepared from compounds of general formula (XI) by process step (xi)—Introduction of a suitable protecting group using standard methodology as described in “Protecting Groups in Organic Synthesis” by T. W. Greene and P. Wutz. When PG=trialkylsilyl, such as trimethylchlorosilane or tert-butyldimethylchlorosilane and preferably trimethylchlorosilane, typical conditions comprise of 1.0 equivalent of compound (XI), 1.1-1.2 equivalents of triethylamine and 1.1-1.2 equivalents of trimethylchlorosilane, in ethyl acetate at 0° C. for 30 minutes.

Compounds of general formula (XIII) can be prepared from compounds of general formula (XII) by process step (xii)—Conversion of alcohol to a suitable leaving group such as mesylate or tosylate by reaction with a sulfonyl chloride such as tosyl chloride or mesyl chloride, in the presence of a suitable base such as triethylamine or pyridine, in a suitable solvent such ethyl acetate or diethyl ether, at ambient temperature for 30-60 minutes. Typical conditions comprise of 1.0 equivalent of compound (XII), 1.1-1.2 equivalents of triethylamine and 1.1-1.2 equivalents of methanesulfonyl chloride, in ethyl acetate at room temperature for 30 minutes.

Compounds of general formula (XIV) can be prepared from compounds of general formula (XIII) by process step (xiii)—De-protection of compound (XIII) may be achieved using standard methodology as described in “Protecting Groups in Organic Synthesis” by T. W. Greene and P. Wutz. When PG′=TMS, typical conditions comprise of 1.0 equivalent of compound (XIII) and an excess of dilute hydrochloric acid in ethyl acetate, at room temperature for 30 minutes.

Compounds of general formula (XV) can be prepared from compounds of general formula (XIV) by process step (xiv)—Epoxidation in the presence of a suitable base such as concentrated sodium or potassium hydroxide solution and a phase transfer catalyst such as methyltri-n-butylammonium chloride or tetrabutyl ammonium chloride, in a suitable solvent such as toluene or xylene at ambient temperature for 30-60 minutes. Typical conditions comprise of 1.0 equivalent of compound (XIV), 4.0-5.0 equivalents of 5M sodium hydroxide solution and methyltri-n-butylammonium (cat) in toluene, at 25° C. for 30 minutes.

Compounds of general formula (XVI) can be prepared from compounds of general formula (XV) by process step (xv)—Reaction with ammonium hydroxide solution, in a suitable solvent such as methanol or ethanol, at elevated temperature for 12-48 hours. Typical conditions comprise of 1.0 equivalent of compound (XV) and excess of ammonium hydroxide solution in methanol for 48 hours at 40° C.

Compounds of general formula (XVII) can be prepared from compounds of general formula (XVI) by process step (iii) as described in Scheme 1.

Compounds of general formula (XVIII) can be prepared from compounds of general formula (XVII) by process step (iv) as described in Scheme 1.

Compounds of general formula (I) can be prepared from compounds of general formula (XVIII) by process step (vi) as described in Scheme 1.

Scheme 3 shows the route to the diasteroisomer, (R*S) but a man skilled in the art will appreciate that this route is also applicable to the isolation of the (R*R*) diasteroisomer.

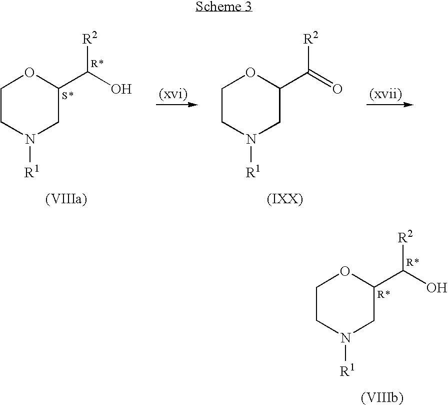

Compounds of general formula (VIIIa) can be prepared as described in Scheme 1.

Compounds of general formula (IXX) can be prepared from compounds of general formula (VIIIa) by process step (xvi)—Reaction with a suitable oxidising agent such as 4-methylmorpholine N-oxide, in the presence of a suitable catalyst such as tetrapropylammonium perruthenate and dehydrating agent such as molecular sieves, magnesium sulfate or sodium sulfate, in a suitable solvent such as dichloromethane or acetonitrile, at ambient temperature for 12-24 hours. Typical conditions comprise of 1.0 equivalent of compound (VIIIa), 1.0-2.0 equivalents of 4-methylmorpholine N-oxide, and tetrapropylammonium perruthenate, in the presence of molecular sieves, in dichloromethane, for 18 hours at room temperature.

Compounds of general formula (VIIIb) can be prepared from compounds of general formula (IXX) by process step (xvii)—Reduction with a suitable selective reducing agent such as zinc borohydride, in a suitable solvent such as diethyl ether or tetrahydrofuran, at ambient temperature for 1-18 hours. Typical conditions comprise of 1.0 equivalent of compound (IXX), 0.3 equivalents of zinc borohydride (generated from 1.0 equivalent of zinc chloride and 2.0 equivalents of sodium borohydride), in diethyl ether at room temperature for 18 hours.

A skilled person will appreciate that compounds of formula I where R¹ is other than hydrogen can be similarly prepared.

Unless otherwise provided herein:
CDI means N,N′-carbonyldiimidazole;
WSCDI means 1-(3-dimethylaminopropyl)-3-ethylcarbodiimide hydrochloride;
DCC means N,N′-dicyclohexylcarbodiimide;
HOAT means 1-hydroxy-7-azabenzotriazole;
HOBT means 1-hydroxybenzotriazole hydrate;
Hünig's base means N-ethyldiisopropylamine;
Et₃N means triethylamine;
NMM means N-methylmorpholine;
DIBAL means diisobutylammonium hydride;
Dess-Martin periodinane means 1,1,1-triacetoxy-1,1-dihydro-1,2-benziodoxol-3(1H)-one;
BSA means N,O-Bis(trimethylsilyl)acetamide;
Boc means tert-butoxycarbonyl;
CBz means benzyloxycarbonyl;
MeOH means methanol;
EtOH means ethanol;
EtOAc means ethyl acetate;
THF means tetrahydrofuran;
DMSO means dimethyl sulphoxide;
DCM means dichloromethane;
DMF means N,N-dimethylformamide;
ACOH means acetic acid; and
TFA means trifluoroacetic acid.

Certain intermediates described above are novel compounds and it is to be understood that all novel intermediates herein are to be considered as further aspects of the present invention.

Racemic compounds may be separated either using preparative HPLC and a column with a chiral stationary phase, or resolved to yield individual enantiomers utilizing methods known to those skilled in the art. In addition, chiral intermediate compounds may be resolved and used to prepare chiral compounds of the invention.

The compounds of the invention may have the advantage that they are more potent, have a longer duration of action, have a broader range of activity, are more stable, have fewer side effects or are more selective, or have other more useful properties than the compounds of the prior art.

The compounds of the invention are useful because they have pharmacological activity in mammals, including humans. Thus, they are useful in the treatment or prevention of disorders in which the regulation of monoamine transporter function is implicated, more particularly disorders in which inhibition of re-uptake of serotonin or noradrenaline is implicated, and especially those in which inhibition of serotonin and noradrenaline re-uptake is implicated.

Accordingly the compounds of the invention are useful in the treatment of urinary incontinence, such as genuine stress incontinence (GSI), stress urinary incontinence (SUI) or urinary incontinence in the elderly; overactive bladder (OAB), including idiopathic detrusor instability, detrusor overactivity secondary to neurological diseases (e.g. Parkinson's disease, multiple sclerosis, spinal cord injury and stroke) and detrusor overactivity secondary to bladder outflow obstruction (e.g. benign prostatic hyperplasia (BPH), urethral stricture or stenosis); nocturnal eneuresis; urinary incontinence due to a combination of the above conditions (e.g. genuine stress incontinence associated with overactive bladder); and urinary symptoms, such as frequency and urgency.

The compounds are also useful in the treatment of faecal incontinence.

In view of their aforementioned pharmacological activity the compounds of Formula Ia and Ib are also useful in the treatment of depression, such as major depression, recurrent depression, single episode depression, subsyndromal symptomatic depression, depression in cancer patients, depression in Parkinson's patients, postmyocardial infarction depression, paediatric depression, child abuse induced depression, depression in infertile women, post partum depression, premenstrual dysphoria and grumpy old man syndrome.

Additionally, the compounds of the invention are useful in the treatment of patients suffering from depression or anxiety with one or more concomitant condition, disease or disorder, or from post traumatic stress disorder. Said condition, disease or disorder concomitant with depression includes, but is not limited to, anxiety and sleep disorders including insomnia, alone or in combination.

The condition, disease or disorder can be selected from: generalized anxiety disorder, major depressive disorder, dysthymia, premenstrual dysphoric disorder, depression with concomitant anxiety, post traumatic stress disorder, panic disorder, specific phobias, obsessive compulsive disorder (OCD), borderline personality disorder, sleep disorders including insomnia, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, deteriorated cerebral function in geriatric patients, chemical dependencies, premature ejaculation, premenstrual syndrome (PMS) associated mood and appetite disorder, hot flashes, cancer, post myocardial infarction, regulation of immune response, immune system disorders, prevention of stenosis, modification of feeding behavior, blocking carbohydrate cravings, late luteal phase dysphoric disorder, attention deficit hyperactivity disorder (ADHD) with or without comorbid anxiety, tobacco withdrawal-associated symptoms, circadian rhythm disorders, psychoactive substance abuse and dependence, schizophrenia, paraphilias, sexual dysfunctions, stress related illnesses and personality disorders manifested by anger, rejection sensitivity, low mental or physical energy, circadian rhythm disorders, personality disorders including borderline and antisocial personality disorders, hyopochondriasis, late luteal phase dysphoric disorder, psychoactive substance use disorders, sexual disorders, and schizophrenia, and related symptoms including stress, worry, lack of mental or physical energy, somatoform disorders, somatization disorder, conversion disorder, body dysmorphic disorder; glaucoma, or ocular hypertension, senile dementia and other forms of memory impairment, neurodegenerative diseases, amyotrophic lateral sclerosis, cerebellar dysfunction, glutamate neurotoxicity in pathophysiology of spinal cord injury induced by aortic cross-clamping, neurological lesions related to traumatic injuries, especially spinal, cranial or cranial-spinal injuries, mitochondrial diseases, including Kearns-Sayre syndrome, MERRF syndrome, MELAS syndrome and Leber's disease, cerebrovascular disorders, neuro-AIDs including disorders involving dementia, cognitive disorders, myopathies, ocular disorders and all neurological symptoms associated with the HIV-1 virus, the cough that is observed in patients who are being maintained on an ACE inhibitor, benign positional vertigo, inflammatory diseases, physiological conditions associated with the use, or sequelae of use, of cocaine or other psychomotors stimulants, mania in all its various forms whether acute or chronic, single or recurrent, bipolar disorder, phencyclidine (PCP) addiction, addiction to alcohol, cocaine addiction, nicotine addiction, drug-induced, electroshock-induced, light-induced, amygdala-kindled, and audiogenic seizures, perinatal asphyxia, Alzheimer's disease, affective illness including cyclothymia to prevent episodes of cyclothymia, mania with exhibited irritability, distractibility, and poor judgment, bipolar depression, persons predisposed to bipolar disorder to prevent episodes of bipolar disorder, effects of ethanol withdrawal syndrome including tremor, anxiety, attention deficit disorder (ADHD) with or without comorbid anxiety, convulsions, stroke, ischemia (in order to prevent neuronal damage), acute and chronic treatment of obesity, partial onset seizures, primary generalized tonic-clonic seizures, anxiety disorders, such as panic disorder with or without agoraphobia, agoraphobia without history of panic disorder, animal and other phobias, social phobias including the generalized and non-generalized subtypes, obsessive-compulsive disorder, acute stress disorder, generalized or substance-induced anxiety disorder, neuroses, convulsions, and depressive or bipolar disorders, for example single-episode or recurrent major depressive disorder, dysthymic disorder, bipolar I and bipolar II manic disorders, cyclothymic disorder, cardiac disorders such as myocardial infarction, angina, stroke, pulmonary embolism, transient ischemic attack, deep vein thrombosis, thrombotic re-occlusion subsequent to a coronary intervention procedure (heart surgery or vascular surgery), peripheral vascular thrombosis, Syndrome X, heart failure, a disorder in which a narrowing of at least one coronary artery occurs, sleep apneas, depression, seasonal affective disorders and dysthmia, avoidant personality disorder, social phobia; memory disorders including dementia, amnestic disorders and age-associated memory impairment; disorders of eating behavior, including anorexia nervosa and bulimia nervosa, obesity, neuroleptic-induced parkinsonism and tardive dyskinesias, endocrine disorders such as hyperprolactinaemia, vasospasm (particularly in the cerebral vasculature), asthma, atherosclerosis, stuttering, chronic fatigue, alcohol abuse, appetite disorders, weight loss, agoraphobia, amnesia, smoking cessation, nicotine withdrawal syndrome symptoms, depressed mood and/or carbohydrate craving associated with pre-menstrual syndrome, disturbances of mood, disturbances of appetite or disturbances which contribute to recidivism associated with nicotine withdrawal, pre-menstrual dysphoric disorder, trichotillomania, symptoms following discontinuation of antidepressants, aggressive/intermittent explosive disorder, compulsive gambling, compulsive spending, compulsive sex, psychoactive substance use disorder, psychiatric symptoms such as worry, anger, rejection sensitivity, and lack of mental or physical energy, psychoactive substance abuse disorders and obsessive compulsive disorders, abuse of anabolic steroids and dementia of aging either alone or in any combination, or concomitant with depression.

Anxiety disorders include panic disorder with or without agoraphobia, agoraphobia without history of panic disorder, specific phobias including specific animal phobias, social anxiety, social phobia including social anxiety disorder, obsessive-compulsive disorder and related spectrum disorders, stress disorders including post-traumatic stress disorder, acute stress disorder and chronic stress disorder, and generalized anxiety disorders.

In view of their aforementioned pharmacological activity the compounds of the invention are also useful in the treatment of cognitive disorders such as dementia, particularly degenerative dementia (including senile dementia, Alzheimer's disease, Pick's disease, Huntingdon's chorea, Parkinson's disease and Creutzfeldt-Jakob disease) and vascular dementia (including multi-infarct dementia), as well as dementia associated with intracranial space occupying lesions, trauma, infections and related conditions (including HIV infection), metabolism, toxins, anoxia and vitamin deficiency; mild cognitive impairment associated with ageing, particularly age associated memory impairment (AAMI), amnestic disorder and age-related cognitive decline (ARCD); psychotic disorders, such as schizophrenia and mania; anxiety disorders, such as generalised anxiety disorder, phobias (e.g. agoraphobia, social phobia and simple phobias), panic disorder, obsessive compulsive disorder, post traumatic stress disorder and mixed anxiety; personality disorders such as avoidant personality disorder and attention deficit hyperactivity disorder (ADHD); sexual dysfunction, such as premature ejaculation, male erectile dysfunction (MED) and female sexual dysfunction (FSD) (e.g. female sexual arousal disorder (FSAD)); premenstrual syndrome; seasonal affective disorder (SAD); eating disorders, such as anorexia nervosa and bulimia nervosa; obesity; appetite suppression; chemical dependencies resulting from addiction to drugs or substances of abuse, such as addictions to nicotine, alcohol, cocaine, heroin, phenobarbital and benzodiazepines; withdrawal syndromes, such as those that may arise from the aforementioed chemical dependencies; cephalic pain, such as migraine, cluster headache, chronic paroxysmal hemicrania, headache associated with vascular disorders, headache associated with chemical dependencies or withdrawal syndromes resulting from chemical dependencies, and tension headache; pain; Parkinson's diseases, such as dementia in Parkinson's disease, neuroleptic-induced Parkinsonism and tardive dyskinesias); endocrine disorders, such as hyperprolactinaemia; vasospasm, such as in the cerebral vasculature; cerebellar ataxia; Tourette's syndrome; trichotillomania; kleptomania; emotional lability; pathological crying; sleeping disorder (cataplexy); and shock.

In view of their aforementioned pharmacological activity the compounds of the invention are also useful in the treatment of a number of other conditions or disorders, including hypotension; gastrointestinal tract disorders (involving changes in motility and secretion) such as irritable bowel syndrome (IBS), ileus (e.g. post-operative ileus and ileus during sepsis), gastroparesis (e.g. diabetic gastroparesis), peptic ulcer, gastroesophageal reflux disease (GORD, or its synonym GERD), flatulence and other functional bowel disorders, such as dyspepsia (e.g. non-ulcerative dyspepsia (NUD)) and non-cardiac chest pain (NCCP); and fibromyalgia syndrome.

In view of their aforementioned pharmacological activity, the compounds of the invention are also useful in the treatment of pain. For example, pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Further examples include tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertebral discs or abnormalities of the lumber facet joints, sacroiliac joints, paraspinal muscles or the posterior longitudinal ligament

In addition, the compounds of the invention are useful in the treatment of neuropathic pain. This is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, diabetic neuropathy, post herpetic neuralgia, back pain, cancer neuropathy, chemotherapy-induced neuropathy, HIV neuropathy, Phantom limb pain, Carpal Tunnel Syndrome, chronic alcoholism, hypothyroidism, trigeminal neuralgia, uremia, trauma-induced neuropathy, or vitamin deficiencies

Other types of pain include but are not limited to:
Inflammatory pain, such as arthritic pain, including rheumatoid arthritis (RA) and ostoearthritis (OA), and inflammatory bowel disease (IBD);
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis;
Central pain or ‘thalamic pain’ as defined by pain caused by lesion or dysfunction of the nervous system including but not limited to central post-stroke pain, multiple sclerosis, spinal cord injury, Parkinson's disease and epilepsy;
Heart and vascular pain including but not limited to angina, myocardical infarction, mitral stenosis, pericarditis, Raynaud's phenomenon, sclerodoma, skeletal muscle ischemia;
Visceral pain, and gastrointestinal disorders, including the pain associated with dysmenorrhea, pelvic pain, cystitis and pancreatitis;
Head pain including but not limited to migraine, migraine with aura, migraine without aura, cluster headache, tension-type headache; and
Orofacial pain including but not limited to dental pain, temporomandibular myofascial pain.

Disorders of particular interest include incontinence, particulary urinary incontinence such as mixed incontinence, GSI and SUI; pain; fibromyalgia; depression; anxiety disorders, such as obsessive-compulsive disorder and post traumatic stress disorder; personality disorders, such as ADHD; sexual dysfunction; and chemical dependencies and withdrawal syndromes resulting from chemical dependencies.

Thus, according to further aspects, the invention provides:

i) a compound of the invention for use in human or veterinary medicine;

ii) a compound of the invention for use in the treatment of a disorder in which the regulation of monoamine transporter function is implicated, such as urinary incontinence;

iii) the use of a compound of the invention in the manufacture of a medicament for the treatment of a disorder in which the regulation of monoamine transporter function is implicated;

iv) a compound of the invention for use in the treatment of a disorder in which the regulation of serotonin or noradrenaline is implicated;

v) the use of a compound of the invention in the manufacture of a medicament for the treatment of a disorder in which the regulation of serotonin or noradrenaline is implicated;

vi) a compound of the invention for use in the treatment of a disorder in which the regulation of serotonin and noradrenaline is implicated;

vii) the use of a compound of the invention in the manufacture of a medicament for the treatment of a disorder in which the regulation of serotonin and noradrenaline is implicated;

viii) a compound of the invention for use in the treatment of urinary incontinence, such as GSI or SUI;

ix) the use of a compound of the invention in the manufacture of a medicament for the treatment of urinary incontinence, such as GSI or SUI;

x) a compound of the invention for use in the treatment of depression or anxiety;

xi) the use of a compound of the invention in the manufacture of a medicament for the treatment of depression or anxiety;

xii) a method of treatment of a disorder in which the regulation of monoamine transporter function is implicated which comprises administering a therapeutically effective amount of a compound of the invention to a patient in need of such treatment;

xiii) a method of treatment of a disorder in which the regulation of serotonin or noradrenaline is implicated which comprises administering a therapeutically effective amount of a compound of the invention to a patient in need of such treatment;

xiv) a method of treatment of a disorder in which the regulation of serotonin and noradrenaline is implicated which comprises administering a therapeutically effective amount of a compound of the invention to a patient in need of such treatment;

xv) a method of treatment of urinary incontinence, such as GSI or SUI, which comprises administering a therapeutically effective amount of a compound of the invention to a patient in need of such treatment; and

xvi) a method of treatment of depression or anxiety, which comprises administering a therapeutically effective amount of a compound of the invention to a patient in need of such treatment.

It is to be appreciated that all references herein to treatment include curative, palliative and prophylactic treatment, unless explicitly stated otherwise.

The compounds of the invention may be administered alone or as part of a combination therapy. If a combination of therapeutic agents is administered, then the active ingredients may be administered either sequentially or simultaneously in separate or combined pharmaceutical formulations.

Examples of suitable agents for adjunctive therapy include:

an estrogen agonist or selective estrogen receptor modulator (e.g. HRT therapies or lasofoxifene);

an alpha-adrenergic receptor agonist, such as phenylpropanolamine or R-450;

an alpha-adrenergic receptor antagonist (e.g. phentolamine, doxazasin, tamsulosin, terazasin and prazasin), including a selective alpha_{1L}-adrenergic receptor antagonist (e.g. Example 19 of WO98/30560);

a beta-adrenergic agonist (e.g. clenbuterol);

a muscarinic receptor antagonist (e.g. tolterodine or oxybutinin), including a muscarinic M3 receptor antagonist (e.g. darifenacin);

a Cox inhibitor, such as a Cox-2 inhibitor (e.g. celecoxib, rofecoxib, valdecoxib parecoxib or etoricoxib);

a tachykinin receptor antagonist, such as a neurokinin antagonist (e.g. an NK1, NK2 or NK3 antagonist);

a beta 3 receptor agonist;

a 5HT₁ ligand (e.g buspirone);

a 5HT₁ agonist, such as a triptan (e.g. sumatriptan or naratriptan);

a dopamine receptor agonist (e.g. apomorphine, teachings on the use of which as a pharmaceutical may be found in U.S. Pat. No. 5,945,117), including a dopamine D2 receptor agonist (e.g. premiprixal, Pharmacia Upjohn compound number PNU95666; or ropinirole);

a melanocortin receptor agonist (e.g. melanotan II);

a PGE receptor antagonist;

a PGE1 agonist (e.g. alprostadil);

a further monoamine transport inhibitor, such as an noradrenaline re-uptake inhibitor (e.g. reboxetine), a serotonin re-uptake inhibitor (e.g. sertraline, fluoxtine, or paroxetine), or a dopamine re-uptake Inhibitors;

a 5-HT3 receptor antagonist (e.g. ondansetron, granisetron, tropisetron, azasetron, dolasetron or alosetron);

a phosphodiesterase (PDE) inhibitor, such as PDE2 inhibitor, (e.g. erythro-9-(2-hydroxyl-3-nonyl)-adenine or Example 100 of EP 0771799, incorporated herein by reference) and in particular a PDE5 inhibitor (e.g. sildenafil; 1-{[3-(3,4-dihydro-5-methyl-4-oxo-7-propylimidazo[5,1-f]-as-trazin-2-yl)-4-ethoxyphenyl]sulfonyl}-4-ethylpiperazine, i.e. vardenafil, also known as Bayer BA 38-9456; or Icos Lilly's IC351, see structure below).

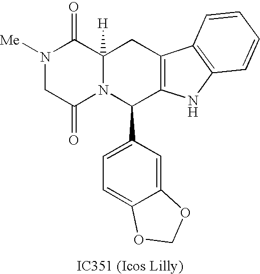

The compounds of the present invention may also be administered as part of a combination therapy for the treatment of fibromyalgia with one or more agents useful for treating one or more indicia of fibromyalgia selected from the group consisting of: non-steroidal anti-inflammatory agents (hereinafter NSAID's) such as piroxicam, loxoprofen, diclofenac, propionic acids such as naproxen, flurbiprofen, fenoprofen, ketoprofen and ibuprofen, ketorolac, nimesulide, acetominophen, fenamates such as mefenamic acid, indomethacin, sulindac, apazone, pyrazolones such as phenylbutazone, salicylates such as aspirin, COX-2 inhibitors such as CELEBREX® (celecoxib), and etoricoxib: steroids, cortisone, prednisone, NEURONTIN®, LYRICA®, muscle relaxants including cyclobenzaprine and tizanidine; hydrocodone, dextropropoxyphene, lidocaine, opioids, morphine, Fentanyl, tramadol, codeine, Paroxetine (PAXIL®), Diazepam, Femoxetine, Carbamazepine, Milnacipran (IXEL®), Vestra®, Venlafaxine (EFFEXOR®)), Duloxetine (CYMBALTA®), Topisetron (NAVOBAN®), Interferon alpha (Veldona), Cyclobenzaprine, CPE-215, Sodium oxbate (XYREM®), Celexa™ (citalopram HBr), ZOLOFT® (sertraline HCl), antidepressants, tricyclic antidepressants, Amitryptyline, Fluoxetine (PROZAC®), topiramate, escitalopram, benzodiazepenes including diazepam, bromazepam and tetrazepam, mianserin, clomipramine, imipramine, topiramate, and nortriptyline.

The invention thus provides, in a further aspect, a combination comprising a compound of the invention together with a further therapeutic agent.

For human use the compounds of the invention can be administered alone, but in human therapy will generally be administered in admixture with a suitable pharmaceutical excipient, diluent or carrier selected with regard to the intended route of administration and standard pharmaceutical practice.

For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via intracavernosal injection. The compounds of the invention may also be administered via fast dispersing or fast dissolving dosage forms.

Such tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention, and their pharmaceutically acceptable salts, may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including PE), compounds of the invention may be taken as a single dose on an “as required” basis (i.e. as needed or desired).

Example Tablet Formulation

In general a tablet formulation could typically contain between about 0.01 mg and 500 mg of a compound according to the present invention (or a salt thereof) whilst tablet fill weights may range from 50 mg to 1000 mg. An example formulation for a 10 mg tablet is illustrated:

| Ingredient | % w/w |
|---|---|
| Free base or salt of compound | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity and is based on the weight of the free base.

The compounds of the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trichlorofluoromethane, dichlorotetra-fluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insulator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the ocular, pulmonary or rectal routes.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

For oral or parenteral administration to human patients the daily dosage levels of compounds of formula (I), and their pharmaceutically acceptable salts, will be from 0.01 to 30 mg/kg (in single or divided doses) and preferably will be in the range 0.01 to 5 mg/kg. Thus tablets will contain 1 mg to 0.4 g of compound for administration singly or two or more at a time, as appropriate. The physician will in any event determine the actual dosage which will be most suitable for any particular patient and it will vary with the age, weight and response of the particular patient. The above dosages are, of course only exemplary of the average case and there may be instances where higher or lower doses are merited, and such are within the scope of the invention.

Oral administration is preferred.

For veterinary use, a compound of the invention is administered as a suitably acceptable formulation in accordance with normal veterinary practice and the veterinary surgeon will determine the dosing regimen and route of administration which will be most appropriate for a particular animal.

Thus according to a further aspect, the invention provides a pharmaceutical formulation containing a compound of the invention and a pharmaceutically acceptable adjuvant, diluent or carrier.

The combinations referred to above may also conveniently be presented for use in the form of a pharmaceutical formulation and thus pharmaceutical formulations comprising a combination as defined above together with a pharmaceutically acceptable adjuvant, diluent or carrier comprise a further aspect of the invention. The individual components of such combinations may be administered either sequentially or simultaneously in separate or combined pharmaceutical formulations. When a compound of the invention is used in combination with a second therapeutic the dose of each compound may differ from that when the compound is used alone. Appropriate doses will be readily appreciated by those skilled in the art.

The invention is illustrated by the following non-limiting examples in which the following abbreviations and definitions may be used:
APCI Atmospheric pressure chemical ionisation
Arbacel® filter agent
br Broad
BOC tert-butoxycarbonyl
CDI carbonyldiimidazole
[58 ] chemical shift
d doublet
□ heat
DCCl dicyclohexylcarbodiimide
DCM dichloromethane
DMF N,N-dimethylformamide
DMSO dimethylsulfoxide
ES⁺ electrospray ionisation positive scan
ES⁻ electrospray ionisation negative scan
h hours
HOAT 1-hydroxy-7-azabenzotriazole
HOBT 1-hydroxybenzotriazole
HPLC high pressure liquid chromatography
m/z mass spectrum peak
min minutes
MS mass spectrum
NMM N-methyl morpholine
NMR nuclear magnetic resonance
q quartet
s singlet
t triplet
TBTU 2-(1H-benzotriazol-1-yl)-1,1,3,3-tetramethyluronium tetrafluoroborate
Tf trifluoromethanesulfonyl
TFA trifluoroacetic acid
THF tetrahydrofuran
TLC thin layer chromatography
TS⁺ thermospray ionisation positive scan
WSCDI 1-(3-dimethylaminopropyl)-3-ethylcarbodiimide hydrochloride

The Preparations and Examples that follow illustrate the invention but do not limit the invention in any way. All temperatures are in ° C. For the Preparations 1-79 and Examples 1-36 the following was used: Flash column chromatography was carried out using Merck silica gel 60 (9385). Solid Phase Extraction (SPE) chromatography was carried out using Varian Mega Bond Elut (Si) cartridges (Anachem) under 15 mmHg vacuum. Thin layer chromatography (TLC) was carried out on Merck silica gel 60 plates (5729). Melting points were determined using a Gallenkamp MPD350 apparatus and are uncorrected. NMR was carried out using a Varian-Unity Inova 400 MHz nmr spectrometer or a Varian Mercury 400 MHz nmr spectrometer. Mass spectroscopy was carried out using a Finnigan Navigator single quadrupole electrospray mass spectrometer or a Finnigan aQa APCI mass spectrometer.

Conveniently, compounds of the invention are isolated following work-up in the form of the free base, but pharmaceutically acceptable acid addition salts of the compounds of the invention may be prepared using conventional means. Solvates (e.g. hydrates) of a compound of the invention may be formed during the work-up procedure of one of the aforementioned process steps.

Where compounds were prepared in the manner described for an earlier Example, the skilled person will appreciate that it may nevertheless be necessary or desirable to employ different work-up or purification conditions.

### Preparation 1

### 4-(4-Methoxybenzyl)morpholin-3-one

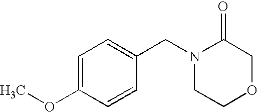

Ethanolamine (22.42 g, 367 mmol) was added to a solution of p-methoxybenzaldehyde (50 g, 367 mmol) in methanol (500 mL) and the solution was stirred at 20° C. for 16 hours. The reaction mixture was then evaporated under reduced pressure to give a viscous orange oil. Platinum oxide (6.5 g, 28.6 mmol) was added to a solution of this oil dissolved in methanol (1 L), and the mixture was stirred under 30 psi of hydrogen gas for 4 hours. The reaction mixture was then filtered through Celite, washing through with methanol, and the filtrate was concentrated in vacuo to give a colourless oil. This oil was dissolved in a mixture of dichloromethane (200 mL) and water (500 mL) and solutions of chloroacetyl chloride (137.4 g, 1.22 mol) in dichloromethane (600 mL), and sodium hydroxide (48.62 g, 1.22 mol) in water (500 mL) were added simultaneously over 2 hours using dropping funnels. Throughout the addition the temperature of the reaction was maintained at 20° C. with an ice-bath. After stirring for 1 hour, the aqueous layer was separated and extracted with dichloromethane (2×400 mL). The combined organic extracts were washed with 1 M sodium hydroxide solution, 2M hydrochloric acid, water and brine. The organic phase was then dried over magnesium sulfate and evaporated under reduced pressure to give a yellow liquid. This liquid was dissolved in methanol (2.1 L) and potassium hydroxide (98.4 g, 1.76 mol) was added portionwise. The resulting suspension was stirred at 20° C. for 6 hours and was then filtered, washing through with methanol. The filtrate was evaporated under reduced pressure and the residue was partitioned between hydrochloric acid (0.5M, 600 mL) and dichloromethane (600 mL). The organic layer was separated, dried over magnesium sulfate and concentrated in vacuo. Re-crystallisation of the residue from hot cyclohexane/ethyl acetate afforded the title compound as a colourless solid in 65% yield, 158.8 g. ¹HNMR (CDCl₃, 400 MHz) δ: 3.21 (m, 2H), 3.77 (s, 3H), 3.79 (m, 2H), 4.19 (s, 2H), 4.52 (s, 2H), 6.83 (d, 2H), 7.17 (d, 2H). MS ES⁺ m/z 222 [MH]⁺.

### Preparation 2

### N-Benzyl-3-chloro-N-(2-hydroxyethyl)propanamide

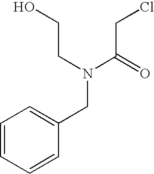

A solution of sodium hydroxide (10.56 g, 264 mmol) in water (200 mL) was added to a solution N-benzylethanolamine (37.6 mL, 263 mmol) in dichloromethane (150 mL). The mixture was cooled to 0° C. and chloroacetyl chloride (20 mL, 264 mmol) was added dropwise over a 3-hour period. The resulting mixture was stirred at room temperature for 18 hours. The mixture was then acidified to pH 2 with 2M hydrochloric acid and the layers were separated. The aqueous layer was extracted with dichloromethane (2×150 mL) and the combined organic extracts were dried over sodium sulfate and concentrated in vacuo. Trituration with diethyl ether afforded the title compound as a white solid in 82% yield, 49.0 g. ¹HNMR (CDCl₃, 400 MHz) δ: 1.22(m, 1H), 3.60 (m, 2H), 4.14 (s, 2H), 4.68 (m, 4H), 7.18-7.42 (m, 5H). MS APCI⁺ m/z 228 [MH]⁺.

### Preparation 3

### 4-Benzylmorpholin-3-one

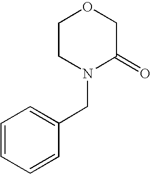

A suspension of potassium hydroxide (12.06 g, 215 mmol) in ethanol (200 mL) was warmed until a solution was formed. The solution was then added to a solution of the product of preparation 2 (49 g, 215 mmol) in ethanol (200 mL) and the mixture was stirred at room temperature for 90 hours. Additional potassium hydroxide (2.41 g, 43 mmol) in ethanol (20 mL) was then added and the mixture was sonicated for 30 minutes. The mixture was then filtered, washing through with ethyl acetate, and the filtrate was evaporated under reduced pressure. The residue was dissolved in ethyl acetate and washed with water and the aqueous layer was re-extracted with ethyl acetate (×2). The combined organic solutions were dried over sodium sulfate and concentrated in vacuo to afford the title product as a pale yellow oil in 81% yield, 41.16 g. ¹HNMR (CDCl₃, 400 MHz) δ: 3.27(m, 2H), 3.83 (m, 2H), 4.27 (s, 2H), 4.63 (s, 2H), 7.22-7.40 (m, 5H). MS APCI⁺ m/z 192 [MH]⁺.

### Preparations 4 and 5

n-Butyl lithium (2.5M in hexane, 4.32 mL, 10.8 mmol) was added to an ice-cold solution of diisopropylamine (1.65 mL, 11.7 mmol) in tetrahydrofuran (6 mL) and the mixture was stirred for 30 minutes, allowing the temperature to rise to 25° C. The reaction mixture was then cooled to −78° C. and a solution of the product of preparation 1 (2 g, 9 mmol) in tetrahydrofuran (18 mL) was added dropwise. The reaction mixture was stirred for 30 minutes, maintaining an internal temperature of below −70° C. 4-Fluorobenzaldehyde (1.21 mL, 11.25 mmol) was added dropwise and the mixture was stirred for a further hour at −78° C. The reaction was then quenched with isopropanol (5 mL) and allowed to warm to −30° C., whereupon ammonium chloride solution (25 mL) was added. The resulting precipitate was dissolved with the addition of 2M hydrochloric acid and the reaction mixture was extracted with diethyl ether (3×100 mL). The combined organic layers were dried over sodium sulfate and concentrated in vacuo to give a viscous brown oil. Purification of the oil by column chromatography on silica gel, eluting with ethyl acetate:pentane, 33:66 to 66:33, firstly afforded the compound of preparation 4 as a white solid in 14% yield, 426 mg. Further elution then afforded the compound of preparation 5 in 18% yield, 546 mg.

### Preparation 4

### (2S*)-2-[(1R*)-(4-Fluorophenyl)(hydroxy)methyl]-4-(4-methoxybenzyl)morpholin-3-one

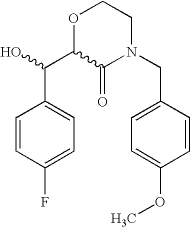

¹HNMR (CDCl₃, 400 MHz) δ: 2.90(d, 1H), 3.16 (m, 1H), 3.73 (m, 1H), 3.77 (s, 3H), 3.96 (m, 1H), 4.19 (d, 1H), 4.50 (d, 1H), 4.70 (d, 1H), 5.10 (m, 1H), 6.79 (d, 2H), 6.87 (d, 2H), 7.00 (m, 2H), 7.42 (m, 2H). MS APCI⁺ m/z 345 [MH]⁺.

### Preparation 5

### (2R)-2-[(1R′)-(4-Fluorophenyl)(hydroxy)methyl]-4-(4-methoxybenzyl)morpholin-3-one

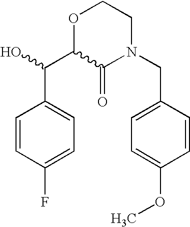

¹HNMR (CDCl₃, 400 MHz) δ: 3.03(d, 1H), 3.36 (m, 1H), 3.62 (m, 1H), 3.81 (s, 3H), 3.89 (m, 1H), 4.18 (d, 1H), 4.56 (d, 2H), 4.94 (d, 1H), 6.85 (m, 2H), 7.01 (m, 2H), 7.10 (d, 2H), 7.40 (m, 2H) MS APCI⁺ m/z 345 [MH]⁺

### Preparations 6 to 11

The following compounds of the general formula shown below were prepared from the product of preparation 1 and the appropriate aldehyde, using a similar method to that described for preparations 4 and 5.

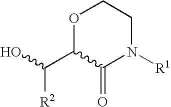

The diastereoisomers were separated using the chromatography conditions described for preparation 4 and 5. Table 1 represents compounds with (1R*,2S*) relative stereochemistry and Table 2 represents compounds with (1R*,2R*) relative stereochemistry.

TABLE 1
(1R^{*}, 2S^{*})

| No. | R¹ | R² | Data | Yield |
|---|---|---|---|---|
| 6 | 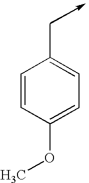 | 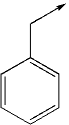 | ¹HNMR(DMSO-D₆, 400 MHz) δ:3.09(d, 1 H), 3.59(m, 1 H), 3.75(m,4 H), 3.95(m, 1 H), 4.27(m, 1 H),4.37(d, 1 H), 4.65(d, 1 H), 5.21(d,1 H), 6.89(d, 2 H), 7.21(m, 3 H),7.30(d, 2 H), 7.40(d, 2 H)MS APCl⁺ m/z 328 [MH]⁺ | 48% |
| 7 | 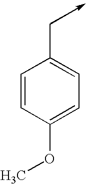 | 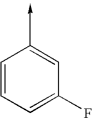 | ¹HNMR(CDCl₃, 400 MHz) δ: 2.92(d,1 H), 3.20(m, 1 H), 3.78(m, 4 H),3.97(m, 1 H), 4.23(d, 1 H), 4.48(d,1 H), 4.52(d, 1 H), 4.70(d, 1 H),5.17(d, 1 H), 6.78(d, 2 H), 6.90(d,2 H), 6.90(m, 1 H), 7.18-7.38(m, 3 H) | 53% |
| 8 | 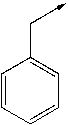 | 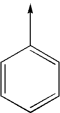 | ¹HNMR(CDCl₃, 400 MHz) δ: 2.94(d,1 H), 3.28(m, 1 H), 3.75(m, 1 H),3.96(m, 1 H), 4.31(d, 1 H), 4.56(d,1 H), 4.79(d, 1 H), 5.21(d, 1 H),6.98(m, 2 H), 7.20-7.40(m, 6 H),7.47(d, 2 H)MS APCl⁺ m/z 298 [MH]⁺ | 57% |

TABLE 2
(1R^{*}, 2R^{*})

| No. | R¹ | R² | Data | Yield |
|---|---|---|---|---|
| 9 | 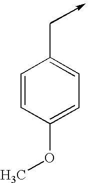 | 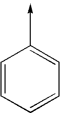 | ¹HNMR(DMSO-D₆, 400 MHz) δ:2.89(m, 2 H), 3.61(m, 1 H), 3.73-4.00(m, 4 H), 4.18(m, 1 H), 4.40(s,1 H), 4.66(d, 1 H), 5.10(m, 1 H),5.54(m, 1 H), 6.79(d, 2 H), 6.90(d, 2 H),7.18-7.38(m, 5 H) | 20% |
| 10 | 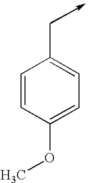 | 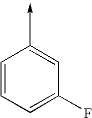 | ¹HNMR(CD₃OD, 400 MHz) δ: 2.92-3.09(m, 2 H), 3.66-3.80(m, 4 H), 3.85-3.94(m, 1 H), 4.04-4.19(m, 1 H),4.53(d, 1 H), 4.70(d, 1 H), 5.24(d, 1 H),6.78(d, 2 H), 6.90(d, 2 H), 7.00(m, 1 H),7.14-7.30(m, 3 H) | 82% |
| 11 | 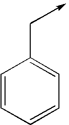 | 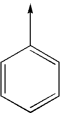 | ¹HNMR(CDCl₃, 400 MHz) δ: 3.05(d,1 H), 3.39(m, 1 H), 3.67(m, 1 H),3.89(m, 1 H), 4.27(d, 1 H), 4.61(s, 2 H),4.99(d, 1 H), 7.18(m, 2 H), 7.22-7.40(m, 6 H), 7.45(d, 2 H)MS APCl⁺ m/z 298 [MH]⁺ | 21% |

### Preparation 12

### (1R*)-(4-Fluorophenyl)[(2S*)-4-(4-methoxybenzyl)morpholin-2-yl]methanol

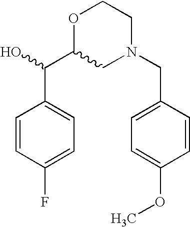

Borane (1 M in tetrahydrofuran, 32.2 mL, 32.3 mmol) was added dropwise to an ice-cold solution of preparation 5 (2.79 g, 8.07 mmol) in tetrahydrofuran (20 mL) and the reaction mixture was stirred at room temperature for 48 hours. Tlc analysis showed that there was still starting material left after this time and so further portions of borane (1M in tetrahydrofuran 8.1 mL, 8.10 mmol) were added at 24-hour intervals, over a 72-hour period. The reaction mixture was then cooled to 0° C., quenched by the careful addition of methanol and evaporated under reduced pressure. The residue was re-dissolved in methanol and the mixture was heated under reflux for 85° C. The reaction mixture was then cooled to room temperature and evaporated under reduced pressure. The residue was partitioned between 1 M sodium hydroxide solution (100 mL) and ethyl acetate (100 mL), and the aqueous layer was re-extracted with ethyl acetate (2×100 mL). The combined organic extracts were dried over sodium sulfate and concentrated in vacuo to give a colourless oil. Purification of the oil by column chromatography on silica gel, eluting with diethyl ether:pentane, 10:90 to 100:0, afforded the title compound in 35% yield, 0.936 g. ¹HNMR (CDCl₃, 400 MHz) δ: 2.18(m, 2H), 2.60 (d, 2H), 3.31 (d, 1H), 3.51 (d, 1H), 3.73 (m, 2H), 3.78 (s, 3H), 3.97 (m, 1H), 5.82 (d, 1H), 6.83 (d, 2H), 7.00 (m, 2H), 7.18 (d, 2H), 7.30 (m, 2H) MS APCI⁺ m/z 332 [MH]⁺.

### Preparations 13 to 19

The following compounds of the general formula shown below were prepared from the appropriate morphilin-3-one, using a similar method to that described for preparation 12. Table 3 represents compounds with (1R*,2R*) relative stereochemistry and Table 4 represents compounds with (1R*,2S*) relative stereochemistry.

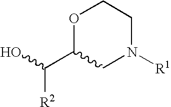

TABLE 3
(1R^{*}, 2R^{*})

| No. | R¹ | R² | Data | Yield |
|---|---|---|---|---|
| 13 | 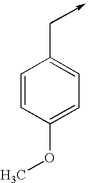 | 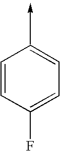 | ¹HNMR(CDCl₃, 400 MHz) δ:2.16(m, 1 H), 2.42(d, 1 H), 2.57(d,1 H), 3.26(d, 1 H), 3.47(d, 1 H),3.64(m, 3 H), 3.78(s, 3 H), 3.94(m,1 H), 4.56(d, 1 H), 6.82(d, 2 H),7.01(m, 2 H), 7.15(d, 2 H), 7.30(m,2 H)MS APCl⁺ m/z 332 [MH]⁺ | Quant. |
| 14 | 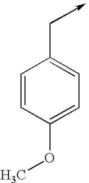 | 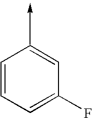 | ¹HNMR(CD₃OD, 400 MHz) δ:1.40(m, 1 H), 1.52(m, 1 H), 1.97(m,1 H), 2.13(m, 1 H), 2.42(d, 1 H),2.60(d, 1 H), 3.30-3.40(m, 2 H),3.78(s, 3 H), 3.89(m, 1 H), 4.58(d,1 H), 6.81(d, 2 H), 6.97(m, 1 H),7.14-7.20(m, 4 H), 7.30(m, 1 H)MS APCl⁺ m/z 332 [MH]⁺ | Quant. |
| 15 | 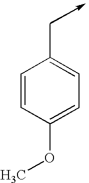 | 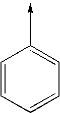 | ¹HNMR(CDCl₃, 400 MHz) δ: 2.02-2.18(m, 2 H), 2.45(d, 1 H), 2.58(d,1 H), 3.24(d, 1 H), 3.50(m, 1 H),3.68(m, 2 H), 3.80(s, 3 H), 3.95(m,1 H), 4.58(d, 1 H), 6.82(d, 2 H),7.17(d, 2 H), 7.22-7.40(m, 5 H),MS APCl⁺ m/z 314 [MH]⁺ | Quant. |
| 16 | 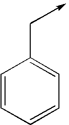 | 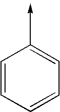 | ¹HNMR(CDCl₃, 400 MHz) δ: 2.00-2.20(m, 2 H), 2.46(m, 1 H), 2.59(m,1 H), 3.30(d, 1 H), 3.54(m, 1 H),3.68(m, 2 H), 3.94(m, 1 H), 4.59(d,1 H), 7.20-7.40(m, 10 H) | 85% |

TABLE 4
(1R^{*}, 2S^{*})

| No | R¹ | R² | Data | Yield |
|---|---|---|---|---|
| 17 | 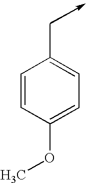 | 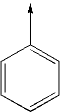 | ¹HNMR(CDCl₃, 400 MHz) δ: 2.10-2.24(m, 2 H), 2.57(m, 2 H), 3.25(d,1 H), 3.41-3.55(m, 1 H), 3.69(m, 2 H),3.80(s, 3 H), 3.99(m, 1 H), 4.88(d,1 H), 6.82(d, 2 H), 7.10-7.40(m, 7 H)MS APCl⁺ m/z 314 [MH]⁺ | Quant. |
| 18 | 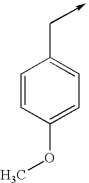 | 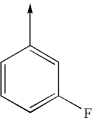 | ¹HNMR(CD₃OD, 400 MHz) δ:2.62(m, 2 H), 2.88(d, 1 H), 3.43(d,2 H), 3.50-3.64(m, 2 H), 3.78(s, 3 H),4.01-4.14(m, 2 H), 4.56(d, 1 H), 6.71-7.03(m, 3 H), 7.03-7.44(m, 5 H)MS APCl⁺ m/z 332 [MH]⁺ | Quant. |
| 19 | 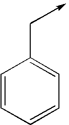 | 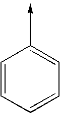 | ¹HNMR(CDCl₃, 400 MHz) δ: 2.14-2.30(m, 2 H), 2.52-2.69(m, 2 H),3.35(d, 1 H), 3.59(d, 1 H), 3.71(m,1 H), 3.82(m, 1 H), 3.96(m, 1 H),4.89(d, 1 H), 7.20-7.40(m, 10 H)MS APCl⁺ m/z 284 [MH]⁺ | Quant |

### Preparation 20

### tert-Butyl {(2S*)-2-[(1R*)-(4-fluorophenyl)(hydroxy)methyl]morpholin-4-yl}acetate

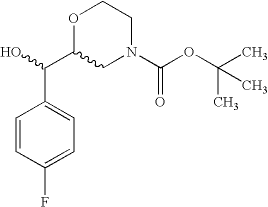

Di-tert-butyl dicarbonate (661 mg, 3.03 mmol), 1-methyl-1,4-cyclohexadiene (1.08 mL, 9.65 mmol) and 10% Pd/C (138 mg) were added to a solution of the product of preparation 12 (0.92 g, 2.78 mmol) in ethanol (14 mL) and the mixture was heated under reflux for 3 hours and at room temperature for 18 hours. The reaction mixture was then filtered through Arbocel®, washing through with ethanol, and the filtrate was concentrated in vacuo. Purification of the residue by column chromatography on silica gel, eluting with pentane:ethyl acetate, 83:17 to 50:50, afforded the title compound as a white solid in 84% yield, 651 mg. ¹HNMR (CDCl₃, 400 MHz) δ: 1.40(s, 9H), 2.77 (m, 1H), 2.90 (m, 1H), 3.53 (m, 2H), 3.76 (m, 2H), 3.90 (m, 1H), 4.84 (m, 1H), 7.04 (m, 2H), 7.31 (m, 2H).

### Preparation 21

### tert-Butyl (2S)-2-[(1R*)-hydroxy(phenyl)methyl]morpholine-4-carboxylate

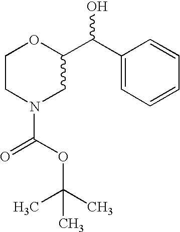

Di-tert-butyl dicarbonate (6.8 g, 31.2 mmol), 1-methyl-1,4-cyclohexadiene (12 mL, 106.8 mmol) and 10% Pd/C (2.5 g) were added to a solution of the product of preparation 17 (9 g, 28.7 mmol) in ethanol (150 mL) and the mixture was heated under reflux for 8 hours and at 60° C. for 18 hours. A further portion of 10% Pd/C (1 g) was then added and the mixture was heated under reflux for 5 hours and at 60° C. for 18 hours. The cooled reaction mixture was then filtered through Arbocel®, washing through with ethanol, and the filtrate was concentrated in vacuo. Purification of the residue by column chromatography on silica gel, eluting with pentane:diethyl ether, 90:10 to 0:100, afforded the title compound as a white solid in quantitative yield.

### Alternative Method

Zinc chloride (1 M in diethyl ether, 50 mL, 50 mmol) was added to a suspension of sodium borohydride (3.7 g, 97.5 mmol) in diethyl ether (200 mL) cooled to 0° C. The mixture was then stirred at 25° C. for 48 hours and then left to stand until the precipitate settled to the bottom of the reaction vessel. A portion (75 mL) of the supernatant layer was removed and added dropwise to an ice-cold solution of the product of preparation 79 (14.3 g, 49.1 mmol) in diethyl ether (100 mL). The mixture was stirred at room temperature for 18 hours and was then cooled to 0° C. Ethyl acetate and ammonium chloride solution (50 mL) were added and the layers were separated. The organic solution was washed with brine and concentrated in vacuo. The residue was purified by column chromatography on silica gel, eluting with ethyl acetate:pentane, 25:75 to 50:50, to afford the title compound as a white solid in 60% yield, 8.65 g. ¹H NMR (CDCl₃, 400 MHz) δ: 1.38 (s, 9H), 2.78-2.97 (m, 2H), 3.45-3.60 (m, 2H), 3.70-3.92 (m, 3H), 4.86 (m, 1H), 7.26-7.40 (m, 5H) MS ES⁺ m/z 316 [MNa]⁺.

### Preparation 22

### tert-Butyl (2S*)-2-[(1R*)-(3-fluorophenyl)(hydroxy)methyl]morpholine-4-carboxylate

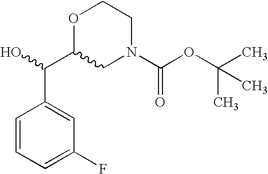

The title compound was prepared from the product of preparation 18, using a similar method to that of preparation 21, as a white solid in 30% yield. ¹HNMR (CDCl₃, 400 MHz) δ: 1.40(s, 9H), 2.50 (m, 1H), 2.80 (m, 1H), 2.91 (m, 1H), 3.48-3.61 (m, 2H), 3.62-3.96 (m, 3H), 4.83 (d, 1H), 6.97 (m, 1H), 7.11 (m, 2H), 7.31 (m, 1H). MS APCI⁺ m/z 312 [MH]⁺.

### Preparation 23

### tert-Butyl {(2R*)-2-[(1R*)-(4-fluorophenyl)(hydroxy)methyl]morpholin-4-yl}acetate

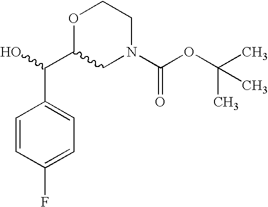

Di-tert-butyl dicarbonate (1.63 g, 7.45 mmol), 1-methyl-1,4-cyclohexadiene (2.66 mL, 23.7 mmol) and 10% Pd/C (340 mg) were added to a solution of the product of preparation 13 (2.25 g, 6.77 mmol) in ethanol (34 mL) and the mixture was heated under reflux for 3 hours and at room temperature for 18 hours. Further portions of di-tert-butyl dicarbonate (295 mg, 1.35 mmol), 1-methyl-1,4-cyclohexadiene (0.76 mL, 6.77 mmol) and 10% Pd/C (68 mg) were then added and the mixture was heated under reflux for 5 hours. The reaction mixture was then cooled to room temperature, filtered through Arbocel®, washing through with ethanol, and the filtrate was concentrated in vacuo. Purification of the residue by column chromatography on silica gel, eluting with pentane:ethyl acetate, 75:25, afforded the title compound as a white solid in 66% yield, 1.39 g. ¹HNMR (CDCl₃, 400 MHz) δ: 1.34(s, 9H), 2.98 (m, 2H), 3.41 (m, 1H), 3.56 (m, 2H), 3.80 (m, 1H), 3.97 (d, 1H), 4.54 (d, 1H), 7.05 (m, 2H), 7.30 (m, 2H). MS APCI⁺ m/z 312 [MH]⁺.

### Preparation 24

### tert-Butyl (2R*)-2-[(1R*)-hydroxy(phenyl)methyl]morpholine-4-carboxylate

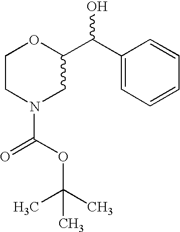

Di-tert-butyl dicarbonate (4 g, 18.3 mmol), 1-methyl-1,4-cyclohexadiene (6.7 mL, 60 mmol) and 10% Pd/C (845 mg) were added to a solution of the product of preparation 15 (5.3 g, 16.9 mmol) in ethanol (85 mL) and the mixture was heated under reflux for 3 hours. The reaction mixture was then cooled to room temperature, filtered through Arbocel®, washing through with ethanol, and the filtrate was concentrated in vacuo. Purification of the residue by column chromatography on silica gel, eluting with pentane:diethyl ether, 60:40 to 0:100, afforded the title compound as a white solid in 67% yield, 3.3 g. ¹HNMR (CDCl₃, 400 MHz) δ: 1.39(s, 9H), 2.62-2.78 (m, 1H), 2.95 (m, 1H), 3.41-3.60 (m, 3H), 3.81 (d, 1H), 3.98 (d, 1H), 4.57 (d, 1H), 7.28-7.40 (m, 5H). MS APCI⁺ m/z 294 [MH]⁺.

### Preparation 25

### tert-Butyl (2R)-2-[(1R*)-(3-fluorophenyl)(hydroxy)methyl]morpholine-4-carboxylate

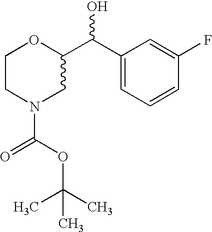

The title compound was prepared from the product of preparation 14, using a similar method to that described for preparation 24, in 90% yield. ¹HNMR (CDCl₃, 400 MHz) δ: 1.38(s, 9H), 2.61-2.76 (m, 1H), 2.83-2.98 (m, 1H), 3.41-3.64 (m, 3H), 3.78 (d, 1H), 3.91 (d, 1H), 4.59 (d, 1H), 7.01 (m, 1H), 7.16 (m, 2H), 7.35 (m, 1H). MS APCI⁺ m/z 312 [MH]⁺.

### Preparation 26

### tert-Butyl (2R)-2-[(1 R*)-(4-chloro-2-methoxyphenoxy)(phenyl)methyl]morpholine-4-carboxylate

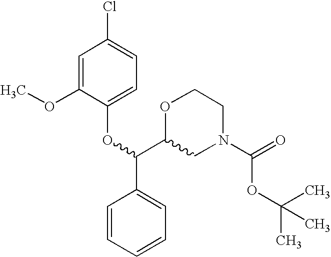

Triphenylphosphine (2.39 g, 9.10 mmol) and 2-methoxy-4-chlorophenol (1.58 mL, 13 mmol) were added to a solution of the product of preparation 21 (1.91 g, 6.50 mmol) in toluene (33 mL). The mixture was cooled to 0° C. and diisopropylazodicarboxylate (1.6 mL, 8.13 mmol) was added dropwise. The reaction mixture was stirred at 0° C. for 30 minutes and at room temperature for 18 hours. The mixture was then diluted with ethyl acetate (350 mL) and washed with 2M sodium hydroxide (2×200 mL) and 10% potassium carbonate solution (200 mL). The organic layer was dried over magnesium sulfate and concentrated in vacuo. The residue was purified by column chromatography on silica gel, eluting with pentane:diethyl ether, 100:0 to 85:15, to afford the title compound as a colourless gum in 76% yield, 2.14 g. ¹HNMR (CDCl₃, 400 MHz) δ: 1.40(s, 9H), 2.77 (m, 1H), 2.95 (m, 1H), 3.56 (m, 2H), 3.83 (m, 5H), 3.96 (m, 1H), 5.09 (d, 1H), 6.65 (m, 2H), 6.79 (d, 1H), 7.26-7.39 (m, 5H). MS APCI⁺ m/z 434 [MH]⁺.

### Preparations 27 to 53

The following compounds of the general formula shown below were prepared from the appropriate BOC-protected morpholine and appropriate phenol using a similar method to preparation 26. The progress of each reaction was monitored by tlc analysis and if required, the reaction mixture was treated with further amounts of diisopropylazodicarboxylate, triphenylphosphine and phenol, at regular intervals, until all of the starting material had been consumed.

Table 5 represents compounds with (1R*,2R*) relative stereochemistry and Table 6 represents compounds with (1R*,2S*) relative stereochemistry.

TABLE 5
(1R^{*}, 2R^{*})

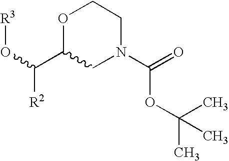

| No | R² | R³ | Data | Yield |
|---|---|---|---|---|
| 27 | 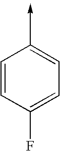 | 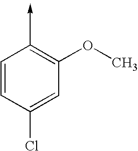 | ¹HNMR(CDCl₃, 400 MHz) δ: 1.41(s,9 H), 2.76(m, 1 H), 2.95(m, 1 H),3.54(m, 1 H), 3.71(m, 2 H), 3.78(m,1 H), 3.80(s, 3 H), 3.96(m, 1 H), 5.07(d,1 H), 6.62(m, 2 H), 6.78(s, 1 H), 7.08(m,2 H), 7.33(m, 2 H)MS APCl⁺ m/z 452 [MH]⁺ | 62% |
| 28 | 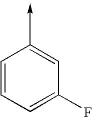 | 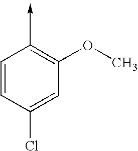 | ¹HNMR(CDCl₃, 400 MHz) δ: 1.41(s,9 H), 2.78(m, 1 H), 2.95(m, 1 H),3.55(m, 1 H), 3.84(m, 6 H), 3.94(d,1 H), 5.09(m, 1 H), 6.61-6.70(m, 2 H),6.80(s, 1 H), 6.97(m, 1 H), 7.12(m, 2 H),7.27(m, 1 H)MS APCl⁺ m/z 452 [MH]⁺ | Quant. |
| 29 | 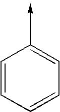 | 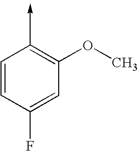 | ¹HNMR(CDCl₃, 400 MHz) δ: 1.40(s,9 H), 2.79(m, 1 H), 2.96(m, 1 H),2.57(m, 1 H), 3.81(m, 6 H), 3.99(d, 1 H)5.14(d, 1 H), 6.39(d, 1 H), 6.58(m, 1 H),6.65(d, 1 H), 7.27-7.40(m, 5 H)MS ES⁺ m/z 440 [MNa]⁺ | 75% |
| 30 | 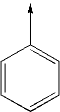 | 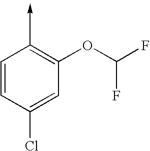 | ¹HNMR(CDCl₃, 400 MHz) δ: 1.40(s,9 H), 2.63-3.03(m, 2 H), 3.49-3.60(m,2 H), 3.75-3.85(m, 2 H), 3.94(d, 1 H),5.25(d, 1 H), 5.49(s, 1 H), 6.96(m, 1 H),7.08(m, 2 H), 7.29-7.46(m, 5 H)MS APCl⁺ m/z 470 [MH]⁺ | 81% |
| 31 | 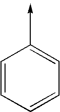 | 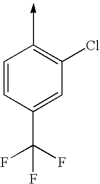 | ¹HNMR(CDCl₃, 400 MHz) δ: 1.42(s,9 H), 2.73(m, 1 H), 2.91(m, 1 H),3.58(m, 1 H), 3.84(m, 4 H), 5.28(d,1 H), 6.62(d, 1 H), 7.25-7.38(m, 6 H),7.59(s, 1 H)MS APCl⁺ m/z 472 [MH]⁺ | 95% |
| 32 | 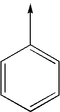 | 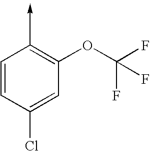 | ¹HNMR(CDCl₃, 400 MHz) δ: 1.38(s,9 H), 2.50(m, 1 H), 2.89(m, 1 H), 3.40-3.60(m, 2 H), 3.72-4.05(m, 3 H),5.22(d, 1 H), 6.98(d, 1 H), 7.08(m, 1 H),7.19(d, 1 H), 7.28-7.42(m, 5 H)MS ES⁺ m/z 510 [MNa]⁺ | 34% |
| 33 | 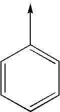 | 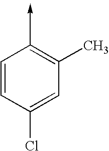 | ¹HNMR(CDCl₃, 400 MHz) δ: 1.41(s,9 H), 2.30(s, 3 H), 2.68(m, 1 H), 2.90(m,1 H), 3.56(m, 1 H), 3.70-3.88(m, 3 H),3.95(m, 1 H), 5.10(d, 1 H), 6.55(d, 1 H),6.90(d, 1 H), 7.07(s, 1 H), 7.22-7.38(m,5 H)MS ES⁺ m/z 440 [MNa]⁺ | 51% |
| 34 | 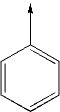 | 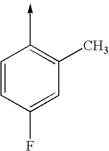 | ¹HNMR(CDCl₃, 400 MHz) δ: 1.42(s,9 H), 2.29(s, 3 H), 2.66(m, 1 H), 2.90(m,1 H), 3.56(m, 1 H), 3.62(m, 2 H),3.73(d, 1 H), 3.95(m, 1 H), 5.05(d, 1 H),6.55(m, 1 H), 6.63(m, 1 H), 6.80(d,1 H), 7.33(m, 5 H)MS APCl⁻ m/z 400 [M − H]⁻ | 48% |
| 35 | 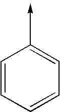 | 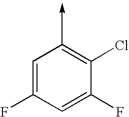 | ¹HNMR(CDCl₃, 400 MHz) δ: 1.40(s,9 H), 2.69(m, 1 H), 2.90(m, 1 H),3.56(m, 1 H), 3.84(m, 4 H), 5.18(d,1 H), 6.35(d, 1 H), 6.45(m, 1 H),7.34(m, 5 H)MS APCl⁺ m/z 440 [MH]⁺ | 85% |
| 36 | 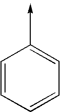 | 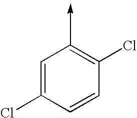 | ¹HNMR(CDCl₃, 400 MHz) δ: 1.41(s,9 H), 2.70(m, 1 H), 2.90(m, 1 H),3.57(m, 1 H), 3.74(d, 1 H), 3.84(m,3 H), 5.18(d, 1 H), 6.78(s, 1 H), 6.81(d,1 H), 7.23(d, 1 H), 7.30-7.40(m, 5 H)MS APCl⁺ m/z 438, 442 [MH]⁺ | 92% |
| 37 | 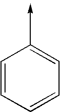 | 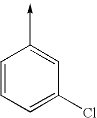 | ¹HNMR(CDCl₃, 400 MHz) δ: 1.41(s,9 H), 2.73(m, 1 H), 2.93(m, 1 H),3.43(m, 1 H), 3.53(m, 1 H), 3.78(m,2 H), 3.93(d, 1 H), 5.10(d, 1 H), 6.74(d,1 H), 6.84(d, 1 H), 6.90(s, 1 H), 7.06(m,1 H), 7.30-7.42(m, 5 H)MS APCl⁻ m/z 402 [M − H]⁻ | 52% |
| 38 | 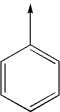 | 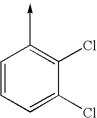 | ¹HNMR(CDCl₃, 400 MHz) δ: 1.41(s,9 H), 2.71(m, 1 H), 2.90(m, 1 H),3.58(m, 1 H), 3.85-3.99(m, 4 H),5.21(d, 1 H), 6.70(d, 1 H), 6.98(m, 2 H),7.27-7.40(m, 5 H)MS APCl⁺ m/z 438 [MH]⁺ | 79% |
| 39 | 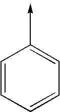 | 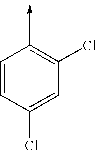 | ¹HNMR(CD₃OD, 400 MHz) δ: 1.40(s,9 H), 2.74(m, 1 H), 2.89(m, 1 H),3.52(m, 1 H), 3.68(d, 1 H), 3.80(m,2 H), 3.92(d, 1 H), 5.38(d, 1 H), 6.90(d,1 H), 7.08(d, 1 H), 7.30-7.42(m, 6 H)MS APCl⁺ m/z 438 [MH]⁺ | 69% |
| 40 | 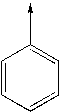 | 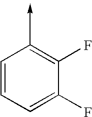 | ¹HNMR(CDCl₃, 400 MHz) δ: 1.40(s,9 H), 2.70(m, 1 H), 2.95(m, 1 H), 3.50-3.70(m, 2 H), 3.77-3.90(m, 2 H),3.96(m, 1 H), 5.16(d, 1 H), 6.58-6.83(m, 3 H), 7.22-7.40(m, 5 H)MS ES⁺ m/z 428 [MNa]⁺ | 88% |
| 41 | 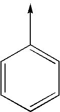 | 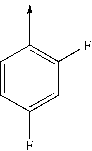 | ¹HNMR(CDCl₃, 400 MHz) δ: 1.40(s,9 H), 2.70(m, 1 H), 2.98(m, 1 H),3.60(m, 2 H), 3.82(m, 2 H), 3.99(m,1 H), 5.04(d, 1 H), 6.60(m, 1 H),6.80(m, 2 H), 7.25-7.40(m, 5 H)MS ES⁺ m/z 428 [MNa]⁺ | Quant. |
| 42 | 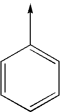 | 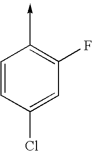 | ¹HNMR(CDCl₃, 400 MHz) δ: 1.40(s,9 H), 2.62(m, 1 H), 2.95(m, 1 H),3.58(m, 2 H), 3.84(m, 2 H), 3.98(m,1 H), 5.09(d, 1 H), 6.77(m, 1 H),6.85(m, 1 H), 7.03(m, 1 H), 7.28-7.40(m, 5 H)MS ES⁺ m/z 444 [MNa]⁺ | 94% |
| 43 | 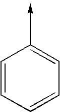 | 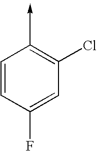 | ¹HNMR(CDCl₃, 400 MHz) δ: 1.42(s,9 H), 2.71(m, 1 H), 2.90(m, 1 H),3.60(m, 1 H), 3.70-4.00(m, 4 H),5.12(d, 1 H), 6.71(m, 2 H), 7.07(m,1 H), 7.21-7.41(m, 5 H)MS APCl⁺ m/z 422 [MH]⁺ | Quant. |
| 44 | 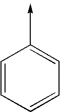 | 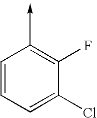 | ¹HNMR(CDCl₃, 400 MHz) δ: 1.40(s,9 H), 2.72(m, 1 H), 2.95(m, 1 H), 3.53-3.70(m, 2 H), 3.83(m, 2 H), 3.98(m,1 H), 5.13(d, 1 H), 6.70-6.85(m, 2 H),6.90(m, 1 H), 7.28-7.40(m, 5 H)MS ES⁺ m/z 444 [MNa]⁺ | 93% |
| 45 | 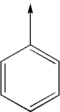 | 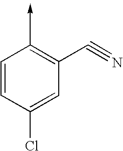 | ¹HNMR(CDCl₃, 400 MHz) δ: 1.40(s,9 H), 2.68(m, 1 H), 2.89(m, 1 H),3.60(m, 2 H), 3.91(m, 3 H), 5.18(m,1 H), 6.80(d, 1 H), 7.22-7.40(m, 6 H),7.49(s, 1 H)MS ES⁺ m/z 451 [MNa]⁺ | Quant. |
| 46 | 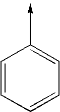 | 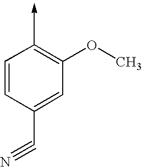 | ¹HNMR(CDCl₃, 400 MHz) δ: 1.40(s,9 H), 2.71(m, 1 H), 2.94(m, 1 H), 3.53-4.00(m, 8 H), 5.20(d, 1 H), 6.73(d, 1 H),7.05(m, 2 H), 7.24-7.40(m, 5 H),MS ES⁺ m/z 447 [MNa]⁺ | 81% |
| 47 | 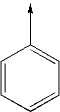 | 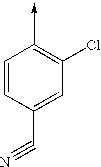 | ¹HNMR(CDCl₃, 400 MHz) δ: 1.41(s,9 H), 2.70(m, 1 H), 2.90(m, 1 H),3.57(m, 1 H), 3.65-3.98(m, 4 H),5.25(d, 1 H), 6.80(d, 1 H), 7.26(s, 1 H),7.30-7.42(m, 5 H), 7.62(s, 1 H)MS APCl⁺ m/z 429 [MH]⁺ | Quant. |
| 48 | 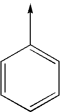 | 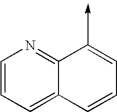 | ¹HNMR(CDCl₃, 400 MHz) δ: 1.39(s,9 H), 2.58(m, 1 H), 3.00(m, 1 H), 3.60-3.90(m, 3 H), 3.99(m, 1 H), 4.16(m,1 H), 5.47(d, 1 H), 6.97(d, 1 H), 7.21-7.38(m, 5 H), 7.42(m, 1 H), 7.50(d,2 H), 8.10(d, 1 H), 9.02(s, 1 H)MS ES⁺ m/z 443 [MNa]⁺ | 56% |
| 49 | 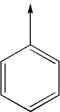 | 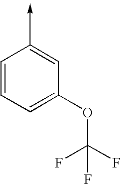 | ¹HNMR(CDCl₃, 400 MHz) δ: 1.40(s,9 H), 2.73(m, 1 H), 2.92(m, 1 H),3.58(m, 2 H), 3.79(m, 2 H), 3.95(d,1 H), 5.10(d, 1 H), 6.76(m, 3 H),7.14(m, 1 H), 7.25-7.40(m, 5 H)MS APCl⁺ m/z 454 [MH]⁺ | 76% |

Preparation 34: Crude product was further purified by additional column chromatography on silica gel, eluting with dichloromethane:methanol:0.88 ammonia to afford title compound

TABLE 6
(1R^{*}, 2S^{*})

| No | R² | R³ | Data | Yield |
|---|---|---|---|---|
| 50 | 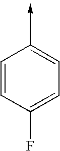 | 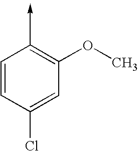 | ¹HNMR(CDCl₃, 400 MHz) δ: 1.44(s,9 H), 2.93(m, 2 H), 3.44(m, 1 H),3.67(m, 2 H), 3.80(s, 3 H), 3.83(m, 1 H),4.39(d, 1 H), 4.96(m, 1 H), 6.53(d, 1 H),6.65(m, 1 H), 6.79(d, 1 H), 6.99(m,2 H), 7.32(m, 2 H)MS APCl⁺ m/z 452 [MH]⁺ | 42% |
| 51 | 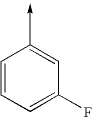 | 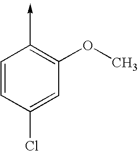 | ¹HNMR(CDCl₃, 400 MHz) δ: 1.45(s,9 H), 2.95(m, 2 H), 3.46(m, 1 H),3.71(m, 1 H), 3.84(s, 5 H), 4.30(d, 1 H),5.00(m, 1 H), 6.57(d, 1 H), 6.67(m,1 H), 6.81(d, 1 H),6.97(m, 1 H),7.14(m, 2 H), 7.30(m, 1 H)MS APCl⁺ m/z 452 [MH]⁺ | Quant. |
| 52 | 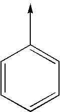 | 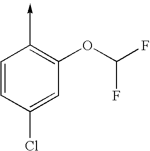 | ¹HNMR(CDCl₃, 400 MHz) δ: 1.41(s,9 H), 2.95-3.08(m, 2 H), 3.50(m, 1 H),3.70(m, 1 H), 3.87(d, 2 H), 4.07(d, 1 H),5.11(m, 1 H), 6.62(m, 2 H), 6.96(d,1 H), 7.15(s, 1 H), 7.29-7.40(m, 5 H)MS APCl⁺ m/z 470 [MH]⁺ | 84% |
| 53 | 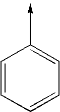 | 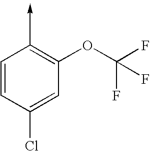 | ¹HNMR(CDCl₃, 400 MHz) δ: 1.41(s,9 H), 2.96(m, 3 H), 3.70(m, 1 H),3.88(m, 1 H), 4.21(m, 2 H), 5.05(d,1 H), 6.68(d, 1 H), 7.01(d, 1 H), 7.19-7.40(m, 6 H)MS APCl⁺ m/z 488 [MH]⁺ | 20% |

### Preparation 54

### 4-Chloro-2-(difluoromethoxy)Phenol

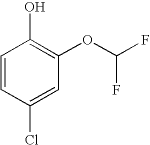

Sulfuryl chloride (2.65 mL, 33 mmol) was added portionwise to a mixture of 2-(difluoromethoxy)phenol (4.9 g, 30.6 mmol), aluminium chloride (31.3 mg, 0.234 mmol) and diphenyl sulfide (5 drops). The reaction mixture was stirred for 18 hours at room temperature to give a dark brown solution. The crude product was then purified by column chromatography on silica gel, eluting with pentane:ethyl acetate, 98:2 to 0:100, to yield some title compound as a colourless oil. The remaining fractions were re-purified by column chromatography on silica gel, eluting with pentane:diethyl ether:ethyl acetate, 90:10:0 to 70:30:0 to 0:0:100, to afford a further amount of title compound giving a combined yield of 62%, 3.72 g. ¹HNMR (CDCl₃, 400 MHz) δ: 5.44(s, 1H) 6.55 (s, 1H), 6.95 (d, 1H), 7.12 (m, 2H).

### Preparation 55

### Methyl 3-chloro-2-methoxybenzoate

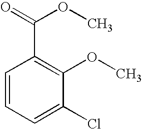

3-Chloro-2-hydroxybenzoic acid (5.5 g, 31.9 mmol) methyl iodide (8.6 mL, 138 mmol) and potassium carbonate (27.5 g, 198 mmol) were suspended in N,N-dimethylformamide (45 mL) and the mixture was heated at 80° C. for 18 hours. Additional methyl iodide (4 mL, 64.2 mmol) was added and the mixture was heated for a further 5 hours at 80° C. The mixture was then cooled to room temperature, diluted with water and extracted with ethyl acetate (×2). The combined organic extracts were washed with water (×2), dried over sodium sulfate and concentrated in vacuo to afford the title compound as a brown oil in quantitative yield, 6.83 g. ¹HNMR (CDCl₃, 400 MHz) δ: 3.95(m, 6H), 7.09 (m, 1H), 7.58 (d, 1H), 7.70 (d, 1H).

### Preparation 56

### Ethyl 4-chloro-2-ethoxybenzoate

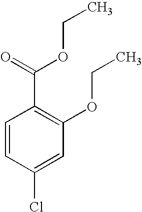

The title compound was prepared from 4-chlorosalicylic acid and ethyl iodide, using a method similar to preparation 55, as an orange oil in 98% yield. ¹HNMR (CDCl₃, 400 MHz) δ: 1.37(t, 3H), 1.48 (t, 3H), 4.09 (q, 2H), 4.34 (q, 2H), 6.95 (m, 2H), 7.72 (d, 1H)

### Preparation 57

### Ethyl 3-chloro-2-ethoxybenzoate

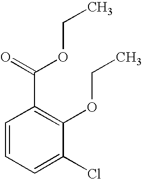

The title compound was prepared from 3-chlorosalicylic acid and ethyl iodide, using a method similar to preparation 55, as a yellow oil in 92% yield. ¹HNMR (CDCl₃, 400 MHz) δ: 1.42(m, 6H), 4.10 (q, 2H), 4.38 (q, 2H), 7.09 (m, 1H), 7.53 (d, 1H), 7.70 (d, 1H)

### Preparation 58

### (3-Chloro-2-methoxyphenyl)methanol

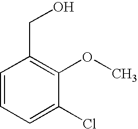

Diisobutylaluminium hydride (1 M in dichloromethane, 70 mL, 70 mmol) was added to a solution of the product of preparation 55 (6.83 g, 34 mmol) in dichloromethane (130 mL) and the mixture was stirred at −78° C. for 45 minutes and at room temperature for 1 hour. Ammonium chloride solution (20 mL) was added portionwise and the mixture was stirred for 5 minutes. 2M Hydrochloric acid (20 mL) was added and the mixture was stirred for a further 5 minutes. The mixture was then stirred over an excess of sodium sulfate for 10 minutes and was filtered, washing through with dichloromethane. The filtrate was concentrated in vacuo to afford the title compound as a yellow oil in 97% yield. ¹HNMR (CDCl₃, 400 MHz) δ: 1.90(brs, 1H), 3.95 (s, 3H), 4.77 (s, 2H), 7.07 (m, 1H), 7.22-7.38 (m, 2H)

### Preparation 59

### (3-Chloro-2-ethoxyphenyl)methanol

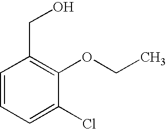

The title compound was prepared from the product of preparation 57 using a method similar to that of preparation 58. Further purification of the crude product by column chromatography on silica gel, eluting with pentane:diethyl ether, 90:10 to 60:40 afforded the title compound as a colourless oil in 91% yield.

¹HNMR (CDCl₃, 400 MHz) δ: 1.46(t, 3H), 1.98 (brs, 1H), 4.10 (d, 2H), 4.72 (s, 2H), 7.05 (m, 1H), 7.24-7.35 (m, 2H). MS ES⁺ m/z 209 [MNa]⁺.

### Preparation 60

### (4-Chloro-2-ethoxyphenyl)methanol

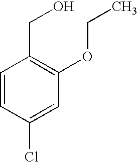

The product of preparation 56 (5.5 g, 24.1 mmol) was added dropwise to an ice-cold solution of lithium aluminium hydride (1M in tetrahydrofuran, 48 mL, 48 mmol) in tetrahydrofuran (30 mL). The mixture was warmed to room temperature and was stirred for 3 hours. The mixture was then re-cooled to 0° C. and water (2 mL), 1M sodium hydroxide solution (2 mL) and water (6 mL) were carefully added. The mixture was diluted with diethyl ether, filtered and the filtrate was concentrated in vacuo to afford the title compound as a white solid in quantitative yield. ¹ HNMR (CDCl₃, 400 MHz) δ: 1.44(t, 3H), 1.62 (s, 1H), 4.08 (q, 2H), 4.65 (s, 2H), 6.82 (s, 1H), 6.92 (d, 1H), 7.19 (d, 1H). MS APCI⁺ m/z 186 [MH]⁺.

### Preparation 61

### 3-Chloro-2-methoxybenzaldehyde

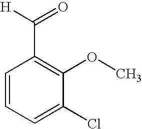

Manganese dioxide (16 g, 184 mmol) was added to a solution of the product of preparation 58 (5.68 g, 33 mmol) in dichloromethane (300 mL) and the mixture was heated for 45° C. for 2.5 hours and at room temperature for 18 hours. The mixture was then filtered through Arbocel®, washing through with dichloromethane, and the filtrate was concentrated in vacuo to afford the title compound as a yellow oil in 92% yield, 5.2 g. ¹HNMR (CDCl₃, 400 MHz) δ: 4.02(s, 3H), 7.19 (m, 1H), 7.63 (d, 1H), 7.79 (d, 1H), 10.40 (s, 1H).

### Preparation 62

### 3-Chloro-2-ethoxybenzaldehyde

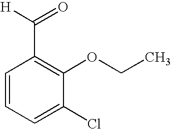

The title compound was prepared from the product of preparation 59, using a similar method to that of preparation 61, as a colourless oil in 91% yield. ¹HNMR (CDCl₃, 400 MHz) δ: 1.48(t, 3H), 4.18 (q, 2H), 7.18 (s, 1H), 7.64 (d, 1H), 7.79 (d, 1H), 10.40 (s, 1H). MSAPCI⁺ m/z 185 [MH]⁺.

### Preparation 63

### 4-Chloro-2-ethoxybenzaldehyde

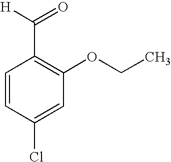

The title compound was prepared from the product of preparation 60, using a similar method to that of preparation 61, as a yellow solid in 73% yield. ¹HNMR (CDCl₃, 400 MHz) δ: 1.44(t, 3H), 4.10 (q, 2H), 7.00 (m, 2H), 7.78 (d, 1H), 10.40 (s, 1H).

### Preparation 64

### 3-Chloro-2-methoxyphenol

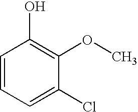

meta-Chloroperbenzoic acid (50-55%, 1.34 g, 40.9 mmol) was added to a solution of the product of preparation 61, (5.2 g, 30.5 mmol) in dichloromethane (120 mL) and the mixture was stirred at room temperature for 18 hours. The reaction mixture was then diluted with dichloromethane and washed with sodium sulphite, sodium hydrogen carbonate solution and evaporated under reduced pressure. The residue was dissolved in methanol (120 mL), triethylamine (0.5 mL) was added, and the mixture was stirred for 18 hours at room temperature. The mixture was then concentrated in vacuo and the residue was dissolved in 1 M sodium hydroxide solution and washed with diethyl ether (×2). The aqueous phase was acidified to pH1 with concentrated hydrochloric acid and extracted with diethyl ether (×2). The combined organic extracts were dried over sodium sulfate and concentrated in vacuo to afford the title compound as a brown oil in 62% yield, 3 g. ¹HNMR (CDCl₃, 400 MHz) δ: 3.98(s, 3H), 6.89-6.99 (m, 3H).

### Preparation 65

### 4-Chloro-2-ethoxyphenol

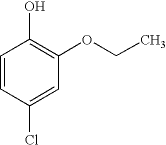

The title compound was prepared from the product of preparation 62, using a similar method to that of preparation 64. Additional purification of the crude compound by column chromatography on silica gel, eluting with pentane:diethyl diethyl ether, 100:0 to 90:10 afforded the title compound as a brown solid in 44% yield. ¹HNMR (CDCl₃, 400 MHz) δ: 1.42(t, 3H), 4.09 (m, 2H), 5.57 (s, 1H), 6.82 (m, 3H). MS APCI⁻ m/z 171 [M−H]⁻

### Preparation 66

### 3-Chloro-2-ethoxyphenol

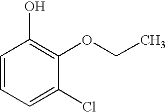

The title compound was prepared from the product of preparation 63, using a similar method to that of preparation 64, as a colourless oil in 86% yield. ¹HNMR (CDCl₃, 400 MHz) δ: 1.42(t, 3H), 4.18 (q, 2H), 5.77 (s, 1H), 6.82-6.97 (m, 3H). MS APCI⁻ m/z 171 [M−H]⁻

### Preparation 67

### tert-Butyl {(2R)-2-[(1R*)-(3-chloro-2-methoxyphenoxy)(phenyl)methyl]morpholin-4-yl}acetate

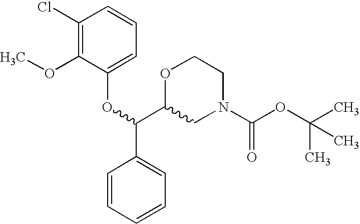

Di-tert-butyl azodicarboxylate (230 mg, 1 mmol) was added portionwise to a solution of the products of preparations 21 (260 mg, 0.9 mmol) and 64 (300 mg, 1.9 mmol), and 4-(diphenylphosphino)pyridine (285 g, 1.03 mmol) in toluene (8 mL) and the mixture was stirred at room temperature for 48 hours. Additional 4-(diphenylphosphino)pyridine (60 mg, 0.23 mmol) and di-tert-butyl azodicarboxylate (50 mg, 0.22 mmol) were then added and the mixture was stirred for an additional 30 minutes. The mixture was then diluted diethyl ether, washed with 1 M sodium hydroxide solution and 2M hydrochloric acid (×2). The organic extract was dried over sodium sulfate and concentrated in vacuo to afford the title compound in quantitative yield.

¹HNMR (CDCl₃, 400 MHz) δ: 1.42(s, 9H), 2.70 (m, 1H), 2.92 (m, 1H), 3.58 (m, 1H), 3.66 (d, 1H), 3.82 (m, 2H), 3.95 (m, 4H), 5.13 (d, 1H), 6.65 (d, 1H), 6.78 (m, 1H), 6.92 (d, 1H), 7.25-7.40 (m, 5H). MS ES⁺ m/z 456 [MNa]⁺

### Preparation 68

### (2R*)-4-Benzyl-2-[(1R*)-(4-chloro-2-ethoxyphenoxy)(phenyl)methyl]morpholine

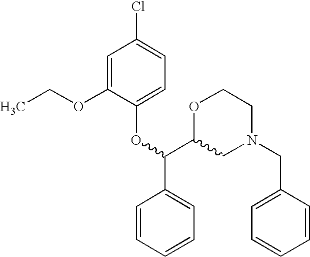

A suspension of the products of preparation 19 (700 mg, 2.47 mmol) and 65 (853 mg, 4.94 mmol), di-tert-butyl azodicarboxylate (851 mg, 4.94 mmol) and tributyl phosphine (1.23 mL, 4.94 mmol) in toluene (20 mL) was heated under reflux for 30 hours and then stirred at room temperature for 60 hours. The reaction mixture was diluted with diethyl ether and washed with 2M sodium hydroxide solution. The organic layer was dried over sodium sulfate and concentrated in vacuo to give a brown oil. The oil was purified by column chromatography on silica gel, eluting with cyclohexane:ethyl acetate, 98:2 to 65:35, to afford the title compound in 40% yield, 404 mg. ¹HNMR (CDCl₃, 400 MHz) δ: 1.39(t, 3H), 2.10 (m, 2H), 2.59 (m, 2H), 3.35 (m, 1H), 3.52 (m, 1H), 3.69 (m, 1H), 3.99 (m, 4H), 5.11 (d, 1H), 6.68 (m, 2H), 6.79 (m, 1H), 7.18-7.40 (m, 10H). MS APCI⁺ m/z 438 [MH]⁺.

### Preparation 69

### (2S*)-4-Benzyl-2-[(1R*)-(4-chloro-2-ethoxyphenoxy)(phenyl)methyl]morpholine

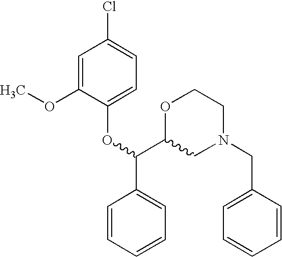

The title compound was prepared from the product of preparation 16 and 2-methoxy-4-chlorophenol, using a method similar to that of preparation 68, as a pale yellow oil in 54% yield. ¹HNMR (CDCl₃, 400 MHz) δ: 2.13-2.30 (m, 2H), 2.60 (m, 2H), 3.19 (m, 1H), 3.43 (m, 1H), 3.60 (m, 2H), 3.78 (s, 3H), 3.83 (d, 1H), 5.02 (d, 1H), 6.58 (d, 1H), 6.65 (d, 1H), 6.80 (s, 1H), 7.20-7.42 (m, 10H).

### Preparation 70

### (2S*)-4-Benzyl-2-[(1R*)-(2,4-dichlorophenoxy)(Phenyl)methyl]morpholine

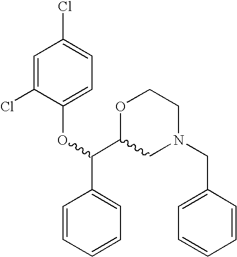

A suspension of the product of preparation 16 (500 mg, 1.75 mmol) and 2,4-dichlorophenol (595 mg, 3.50 mmol), 1,1′-azobis(N,N-dimethylformamide) (600 mg, 3.50 mmol) and tributyl phosphine (0.8 mL, 3.50 mmol) in toluene (10 mL) was heated under reflux for 30 hours and then stirred at room temperature for 60 hours. The reaction mixture was diluted with diethyl ether and washed with 2M sodium hydroxide solution. The organic layer was dried over sodium sulfate and concentrated in vacuo. The residue was purified by column chromatography on silica gel, eluting with cyclohexane:ethyl acetate, 80:20, to afford the title compound as a pale yellow oil in 53% yield, 400 mg. ¹HNMR (CDCl₃, 400 MHz) δ: 2.19(m, 1H), 2.38 (m, 1H), 2.60 (d, 1H), 3.18 (d, 1H) 3.42(d, 1H), 3.60 (m, 2H), 3.80-3.98 (m, 2H), 5.11 (d, 1H), 6.61 (d, 1H), 6.97 (d, 1H), 7.21-7.39 (m, 11H).

### Preparation 71

### (2S)-4-Benzyl-2-[(1R*)-(4-chloro-2-ethoxyphenoxy)(phenyl)methyl]morpholine

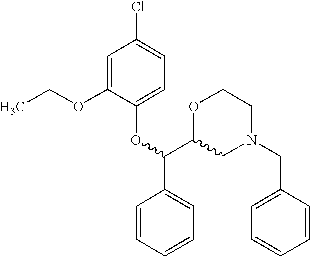

The title compound was prepared from the products of 16 and 65, using a similar method to that of preparation 68, as a colourless oil in 49% yield. ¹HNMR (CDCl₃, 400 MHz) δ: 1.39(t, 3H), 2.15 (m, 1H), 2.30 (m, 1H), 2.61 (m, 1H), 3.21 (m, 1H), 3.43 (m, 1H), 3.62 (m, 1H), 3.82 (m, 1H) 3.97 (m, 4H), 5.01 (d, 1H), 6.57 (d, 1H), 6.64 (d, 1H), 6.79 (s, 1H), 7.22-7.40 (m, 10H). MS APCI⁺ m/z 438 [MH]⁺

### Preparation 72

### (2S*)-4-Benzyl-2-[(1R*)-(3-chloro-2-ethoxyphenoxy)(phenyl)methyl]morpholine

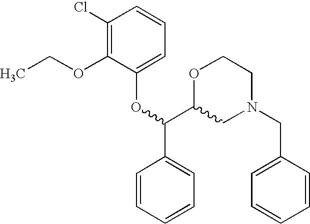

The title compound was prepared from the products of 16 and 66, using a similar method to that of preparation 68, as a colourless oil in 40% yield. ¹HNMR (CDCl₃, 400 MHz) δ: 1.33(t, 3H), 2.18 (m, 1H), 2.32 (m, 1H), 2.64 (m, 1H), 3.06 (m, 1H), 3.46 (m, 1H), 3.60 (m, 2H), 3.80-3.97 (m, 2H) 4.05 (m, 2H), 5.18 (d, 1H), 6.58 (d, 1H), 6.77 (m, 1H), 6.90 (d, 1H), 7.22-7.40 (m, 10H). MS ES⁺ m/z 460 [MNa]⁺.

### Preparation 73

### (2S,3R)-3-(4-Chloro-2-methoxyphenoxy)-3-phenylpropane-1,2-diol

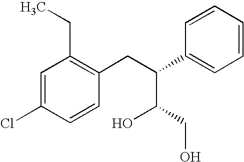

Dichloromethane (30 mL) and tributylmethylammonium chloride (75% in water, 0.5 mL, 5 mol %) were added to a suspension of 4-chloro-2-methoxyphenol (8.1 mL, 66.6 mmol) in 1 M sodium hydroxide solution (30 mL) heated to 60° C. (2S,3S)-3-Phenylglycidol (5 g, 33.3 mmol) in dichloromethane (15 mL) was added dropwise and the mixture was stirred at 40° C. for 2 hours and at 75° C. for 90 minutes. The dichloromethane was distilled off and the reaction mixture was heated at 75° C. for a further 5 hours. The mixture was then diluted with ethyl acetate and washed with 2M sodium hydroxide solution. The organic layer was dried over magnesium sulfate and concentrated in vacuo. Trituration of the residue with a mixture of diethyl ether/pentane afforded the title compound in 61% yield, 6.27 g. ¹HNMR (CDCl₃, 400 MHz) δ: 3.47(m, 2H), 3.70 (m, 1H), 3.89 (s, 3H) 5.22 (d, 1H), 6.52 (d, 1H), 6.67 (d, 1H), 6.86 (s, 1H), 7.30-7.43 (m, 5H). MS APCI⁺ m/z 326 [MNH₄]⁺.

### Preparation 74

### (1S,2R)-2-(4-Chloro-2-methoxyphenoxy)-1-(hydroxymethyl)-2-phenylethyl methanesulfonate

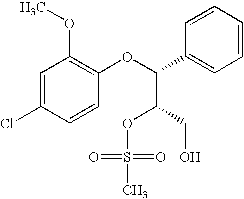

The product of preparation 73 (5.9 g, 19.11 mmol) and triethylamine (3.2 mL, 22.93 mmol) were suspended in ethyl acetate (60 mL) and the mixture was cooled to 0° C. Chlorotrimethylsilane (2.54 mL, 20.07 mmol) was added dropwise and the mixture was stirred at 0° C. for 5 minutes and at room temperature for 25 minutes. The mixture was then re-cooled to 0° C. and methanesulfonyl chloride (1.77 mL, 22.93 mmol) was added dropwise followed by further triethylamine (3.2 mL, 22.93 mmol). The mixture was stirred at 0° C. for 5 minutes and at room temperature for 25 minutes. 1 M Hydrochloric acid was added to the mixture and stirring continued for a further 30 minutes. The mixture was then diluted with ethyl acetate and the organic phase was separated and washed with sodium hydrogen carbonate solution and brine. The organic layer was dried over magnesium sulfate and concentrated in vacuo. The residue was azeotroped with toluene to afford the title compound as a colourless oil in quantitative yield, 7.9 g. ¹HNMR (CDCl₃, 400 MHz) δ: 2.52(s, 3H), 3.75 (s, 3H), 4.00 (m, 2H), 4.82 (m, 1H), 5.19 (d, 1H), 6.42 (d, 1H), 6.58 (d, 1H), 6.75 (s, 1H), 7.20-7.35 (m, 5H). MS APCI⁺ m/z 404 [MNH₄]⁺.

### Preparation 75

### (2R)-2-[(R)-(4-Chloro-2-methoxyphenoxy)(Phenyl)methyl]oxirane

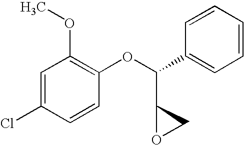

5M Sodium hydroxide solution (17 mL, 85 mmol) and tributylmethylammonium chloride (75% in water, 0.5 mL, 10 mol %) were added to a solution of the product of preparation 74 (7.39 g, 19.11 mmol) in toluene (38 mL) and the mixture was stirred for 30 minutes. The mixture was then diluted with toluene and brine. The organic layer was separated and washed with brine, dried over magnesium sulfate and concentrated in vacuo to afford the title compound as a colourless oil in quantitative yield, 6.7 g. ¹HNMR (CDCl₃, 400 MHz) δ: 2.70(m, 1H), 2.83 (m, 1H), 3.49 (m, 1H), 3.88 (s, 3H), 4.84 (d, 1H), 6.10 (m, 2H), 6.85 (s, 1H), 7.30-7.45 (m, 5H).

### Preparation 76

### (1R,2R)-3-Amino-1-(4-chloro-2-methoxyphenoxy)-1-phenylpropan-2-ol

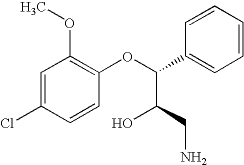

A solution of the product of preparation 75 (6.7 g, 19 mmol) in methanol (45 mL) was added dropwise to concentrated ammonium hydroxide solution over a 10-minute period. The resulting mixture was stirred for 48 hours at room temperature. The mixture was then diluted with a mixture of dichloromethane and methanol (95:5) and loaded onto a column of silica gel. Elution with dichloromethane:ethyl acetate, 100:0 to 0:100, followed by ethyl acetate:methanol:0.88 ammonia, 80:20:2, afforded the title compound as a white solid in 68% yield. ¹HNMR (CDCl₃, 400 MHz) δ: 2.55-2.73 (m, 2H), 3.88 (s, 3H), 3.95 (m, 1H) 4.82 (d, 1H), 6.52 (d, 1H), 6.66 (d, 1H), 6.85 (s, 1H), 7.30-7.42 (m, 5H). MS APCI⁺ m/z 308 [MH]⁺.

### Preparation 77

### 2-Chloro-N-[(2R,3R)-3-(4-chloro-2-methoxyphenoxy)-2-hydroxy-3-phenylpropyl]acetamide

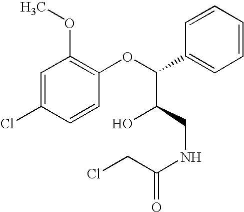

Chloroacetyl chloride (869 μL, 10.91 mmol) in tetrahydrofuran (18 mL) was added dropwise to a solution of the product of preparation 76 (3.8 g, 10.8 mmol) in tetrahydrofuran (36 mL) cooled to −5° C. The mixture was stirred for 20 minutes and was then quenched with water (30 mL) and evaporated under reduced pressure. The residue was taken up in ethyl acetate and washed with water and brine and the organic layer was dried over magnesium sulfate and concentrated in vacuo. The residue was then azeotroped with toluene to afford the title compound in 97% yield, 4.95 g. ¹HNMR (CDCl₃, 400 MHz) δ: 3.25(m, 1H), 3.35 (m, 1H), 3.90 (s, 3H), 4.04 (s, 2H) 4.13 (m, 1H), 4.70 (d, 1H), 6.53 (d, 1H), 6.68 (d, 1H), 6.77 (s, 1H), 7.02 (brs, 1H), 7.32-7.42 (m, 5H). MS APCI⁻ m/z 420 [MCI].

### Preparation 78

### (6R)-6-[(R)-(4-Chloro-2-methoxyphenoxy)(phenyl)methyl]morpholin-3-one

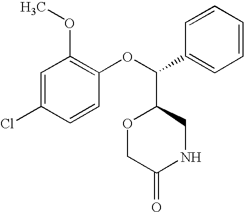

A solution of potassium tert-butoxide (3.24 g, 28.84 mmol) in isopropyl alcohol (30 mL) was added dropwise to an ice-cold solution of the product of preparation 77 (3.96 g, 10.3 mmol) in a mixture of toluene (10 mL) and isopropyl alcohol (20 mL). The reaction mixture was stirred for 1 hour as the temperature rose to room temperature. The mixture was then acidified to pH 6 with 2M hydrochloric acid and the solvent was evaporated under reduced pressure. The aqueous residue was then diluted with toluene (100 mL) and washed with sodium hydrogen carbonate solution and brine. The organic layer was dried over magnesium sulfate and concentrated in vacuo to afford the title compound as a pale brown foam in 88% yield. ¹HNMR (CDCl₃, 400 MHz) δ: 3.00(m, 1H), 3.35 (m, 1H), 3.84 (s, 3H), 4.15-4.22 (m, 1H) 4.31 (m, 2H), 5.18 (d, 1H), 6.30 (brs, 1H), 6.66 (m, 2H), 6.81 (s, 1H), 7.28-7.40 (m, 5H). MS APCI⁺ m/z 348 [MH]⁺.

### Preparation 79

### tert-Butyl 2-benzoylmorpholine-4-carboxylate

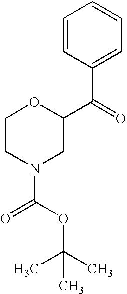

Acetonitrile (50 mL) and 4-methylmorpholine N-oxide (9 g, 76.70 mmol) were added to a solution of the product of preparation 24 (15 g, 51.13 mmol) in dichloromethane (150 mL). Molecular sieves (4 Å, 25 g) were added and the reaction mixture was cooled to 0° C. Tetrapropylammonium perruthenate (720 mg, 4 mol %) was then added portionwise and the mixture was stirred at room temperature for 18 hours. The reaction mixture was filtered twice through a pad of silica, washing through with ethyl acetate, and the combined filtrates were concentrated in vacuo to afford the title compound as a white solid in 96% yield, 14.35 g. ¹HNMR (CDCl₃, 400 MHz) δ: 1.45(s, 9H), 3.07 (m, 2H), 3.70 (m, 1H), 3.87 (d, 1H), 4.03 (m, 1H) 4.22(m, 1H), 4.76 (d, 1H), 7.45 (m, 2H), 7.68 (m, 1H), 8.00 (d, 2H). MS APCI⁺ m/z 314 [MNa]⁺.

### EXAMPLE 1

### (2R*)-2-[(1R*)-(4-Chloro-2-methoxyphenoxy)(phenyl)methyl]morpholine hydrochloride

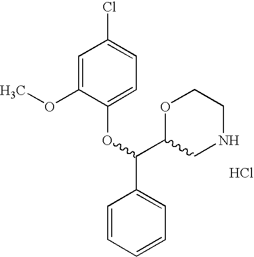

Hydrochloric acid (4M in dioxan, 25 mL) was added to a solution of the product of preparation 26 (2.1 g, 4.84 mmol) in dichloromethane (25 mL) and the mixture was stirred for 18 hours at room temperature. The reaction mixture was then concentrated in vacuo to give a white foam in quantitative yield.

¹HNMR (CD₃OD, 400 MHz) δ: 3.05-3.20 (m, 3H), 3.25 (d, 1H), 3.78-3.87 (m, 4H), 4.08-4.20 (m, 2H), 5.31 (d, 1H), 6.70 (m, 2H), 6.95 (s, 1H), 7.28-7.44 (m, 5H). MS APCI⁺ m/z 334 [MH]⁺.

### EXAMPLES 2 TO 21

The following compounds of general formula shown below were prepared from the appropriate BOC protected starting material, using a similar method to example 1. Table 7 represents compounds with (1R*,2R*) relative stereochemistry and Table 8 represents compounds with (1R*,2S*) relative stereochemistry.

TABLE 7
[(1R^{*}, 2R^{*}) isomers]

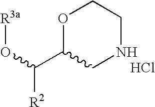

| No | R² | R^{3a} | Data | Yield |
|---|---|---|---|---|
| 2 | 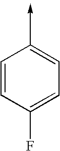 | 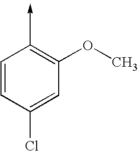 | ¹HNMR(CD₃OD, 400 MHz) δ: 3.09(m,2 H), 3.23(m, 2 H), 3.77(m, 1 H), 3.87(s,3 H), 4.12(m, 2 H), 5.34(d, 1 H), 6.73(m,2 H), 6.96(d, 1 H), 7.09(m, 2 H), 7.42(m,2 H)MS APCl⁺ m/z 352 [MH]⁺Micro analysis found (%); C(54.84),H(5.45), N(3.38);C₁₈H₁₉ClFNO₃.HCl.0.50H₂O requires(%); C(54.45), H(5.33), N(3.53) | 68% |
| 3 | 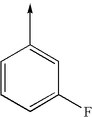 | 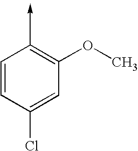 | ¹HNMR(CD₃OD, 400 MHz) δ: 3.13(m,2 H), 3.27(m, 3 H), 3.86(s, 3 H), 4.10-4.16(m, 2 H), 5.36(d, 1 H), 6.74(m, 2 H),6.99(d, 1 H), 7.06(m, 1 H), 7.20(m, 1 H),7.37(m, 2 H)MS APCl⁺ m/z 352 [MH]⁺ | 77% |
| 4 | 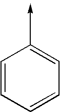 | 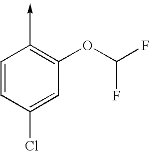 | ¹HNMR(CDCl₃, 400 MHz) δ: 3.00-3.15(m, 3 H), 3.23-3.32(m, 1 H), 3.84(m,1 H), 4.08-4.21(m, 2 H), 5.49(d, 1 H),6.88(m, 2 H), 7.01-7.12(m, 2 H), 7.33-7.48(m, 5 H)MS ES⁺ m/z 370 [MH]⁺ | Quant. |
| 5 | 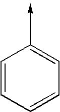 | 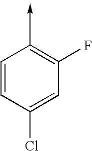 | ¹HNMR(CD₃OD, 400 MHz) δ: 3.00-3.20(m, 3 H), 3.22-3.33(m, 1 H), 3.85(m,1 H), 4.18(m, 2 H), 5.38(d, 1 H), 6.96(m,2 H), 7.18(m, 1 H), 7.30-7.42(m, 5 H)MS ES⁺ m/z 322 [MH]⁺ | 96% |
| 6 | 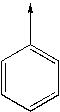 | 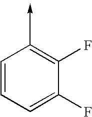 | ¹HNMR(CD₃OD, 400 MHz) δ: 3.03-3.20(m, 3 H), 3.35(m, 1 H), 3.83(m, 1 H),4.10-4.28(m, 2 H), 5.42(d, 1 H), 6.73-6.93(m, 3 H), 7.31-7.48(m, 5 H)MS ES⁺ m/z 306 [MH]⁺Micro analysis found (%); C(57.99),H(5.34), N(3.96);C₁₇H₁₇F₂NO₂.HCl.0.50 H₂O requires(%); C(58.21), H(5.46), N(3.99) | Quant. |
| 7 | 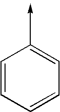 | 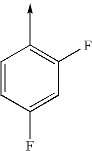 | ¹HNMR(CD₃OD, 400 MHz) δ: 3.00-3.20(m, 3 H), 3.22-3.35(m, 1 H), 3.88(m,1 H), 4.10-4.23(m, 2 H), 5.34(d, 1 H),6.70(m, 1 H), 6.91(m, 2 H), 7.30-7.44(m,5 H)MS ES⁺ m/z 306 [MH]⁺ | Quant. |
| 8 | 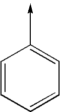 | 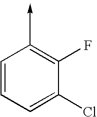 | ¹HNMR(CD₃OD, 400 MHz) δ: 3.01-3.21(m, 3 H), 3.29(m, 1 H), 3.89(m, 1 H),4.17(m, 1 H), 4.23(m, 1 H), 5.42(d, 1 H),6.85-7.00(m, 3 H), 7.30-7.45(m, 5 H)MS ES⁺ m/z 322 [MH]⁺ | 99% |
| 9 | 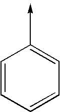 | 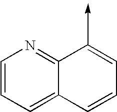 | ¹HNMR(CD₃OD, 400 MHz) δ: 3.08(m,1 H), 3.10-3.28(m, 2 H), 3.35(m, 1 H),4.03(m, 1 H), 4.17(m, 1 H), 4.62(m, 1 H),5.82(d, 1 H), 7.42(m, 3 H), 7.58(d, 1 H),7.63(d, 2 H), 7.78(m, 1 H), 7.89(d, 1 H),8.19(m, 1 H), 9.21(d, 1 H), 9.26(d, 1 H)MS APCl⁺ m/z 321 [MH]⁺ | 99% |
| 10 | 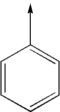 | 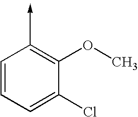 | ¹HNMR(CD₃OD, 400 MHz) δ: 3.02-3.18(m, 3 H), 3.28(m, 1 H), 3.85(m, 1 H),3.91(s, 3 H), 4.14(m, 1 H), 4.22(m, 1 H),5.39(d, 1 H), 6.75(d, 1 H), 6.85(m, 1 H),6.95(d, 1 H), 7.30-7.50(m, 5 H)MS APCl⁺ m/z 334 [MH]⁺ | 90% |
| 11 | 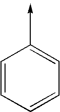 | 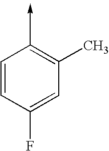 | ¹HNMR(CD₃OD, 400 MHz) δ: 2.30(s,3 H), 2.99(m, 1 H), 3.10(m, 2 H), 3.22(d,1 H), 3.82(m, 1 H), 4.15(m, 2 H), 5.28(d,1 H), 6.64(m, 2 H), 6.83(d, 1 H), 7.37(m,5 H)Micro analysis found (%); C(62.61),H(6.38), N(4.31); C₁₈H₂₀FNO₂.HCl.0.50 H₂O requires(%); C(62.34),H(6.39), N(4.04) | Quant. |
| 12 | 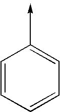 | 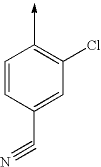 | ¹HNMR(CD₃OD, 400 MHz) δ: 3.10(m,3 H), 3.25(s, 1 H), 3.82(m, 1 H), 4.17(m,1 H), 4.22(m, 1 H), 5.62(d, 1 H), 7.04(d,1 H), 7.38(m, 5 H), 7.48(d, 1 H), 7.80(s,1 H)Micro analysis found (%); C(57.88),H(5.15), N(7.31);C₁₈H₁₇ClN₂O₂.HCl.0.50 H₂O requires(%); C(57.77),H(5.12), N(7.48) | Quant. |
| 13 | 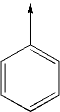 | 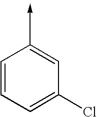 | ¹HNMR(CD₃OD, 400 MHz) δ: 3.09(m,3 H), 3.25(d, 1 H), 3.80(m, 1 H), 4.12(m,2 H), 5.38(d, 1 H), 6.61(d, 1 H), 6.63(m,2 H), 7.14(m, 1 H), 7.38(m, 5 H)Micro analysis found (%); C(58.78),H(5.74), N(4.07);C₁₇H₁₇ClNO₂.HCl.0.50 H₂O requires(%); C(58.63), H(5.50), N(4.02) | Quant. |
| 14 | 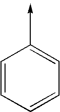 | 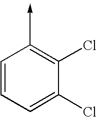 | ¹HNMR(CD₃OD, 400 MHz) δ: 3.02-3.38(m, 4 H), 3.85(m, 1 H), 4.19(m, 2 H),5.52(d, 1 H), 6.85(d, 1 H), 7.06(m, 2 H),7.30-7.42(m, 5 H)MS APCl⁺ m/z 339 [MH]⁺Micro analysis found (%); C(54.20),H(4.99), N(3.78); C₁₇H₁₇Cl₂NO₂.HCl.requires (%); C(54.49), H(4.84),N(3.74) | Quant. |
| 15 | 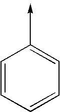 | 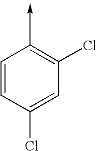 | ¹HNMR(CD₃OD, 400 MHz) δ: 3.01-3.30(m, 4 H), 3.82(m, 1 H), 4.19(m, 2 H),5.50(d, 1 H), 6.89(d, 1 H), 7.09(m, 2 H),7.32-7.42(m, 5 H)MS APCl⁺ m/z 339 [MH]⁺ | Quant. |
| 16 | 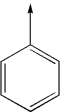 | 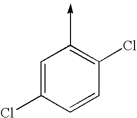 | ¹HNMR(CD₃OD, 400 MHz) δ: 3.09(m,3 H), 3.25(d, 1 H), 3.82(m, 1 H), 4.17(m,2 H), 5.54(d, 1 H), 6.92(m, 2 H), 7.38(m,6 H)Micro analysis found (%); C(54.01),H(5.06), N(3.57); C₁₇H₁₇Cl₂NO₂.HCl0.25 H₂O requires (%); C(53.85),H(4.92), N(3.69) | Quant. |
| 17 | 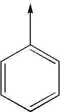 | 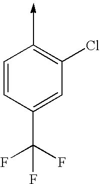 | ¹HNMR(CD₃OD, 400 MHz) δ: 3.10(m,3 H), 3.26(d, 1 H), 3.64(m, 1 H), 4.15(m,1 H), 4.23(m, 1 H), 5.62(d, 1 H), 7.05(d,1 H), 7.40(m, 6 H), 7.68(s, 1 H)Micro analysis found (%); C(52.45),H(4.50), N(3.38);C₁₈H₁₇ClF₃NO₂.HCl.0.25 H₂Orequires(%); C(52.38), H(4.52), N(3.39) | 80% |
| 18 | 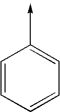 | 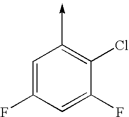 | ¹HNMR(CD₃OD, 400 MHz) δ: 3.08(m,3 H), 3.26(d, 1 H), 3.63(m, 1 H), 4.17(m,2 H), 5.53(d, 1 H), 6.62(m, 1 H), 6.70(m,1 H), 7.41(m, 5 H)Micro analysis found (%); C(54.18),H(4.57), N(3.67); C₁₇H₁₆ClF₂NO₂.HClrequires (%); C(54.27), H(4.55),N(3.72) | Quant. |

TABLE 8
[(1R^{*}, 2S^{*}) isomers]

| No | R² | R^{3a} | Data | Yield |
|---|---|---|---|---|
| 19 | 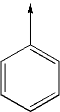 | 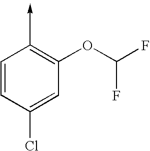 | ¹HNMR(CD₃OD, 400 MHz) δ: 3.10-3.20(m, 1 H), 3.23-3.38(m, 2 H), 3.48(d,1 H), 3.80(m, 1 H), 4.10(m, 2 H), 5.42(d,1 H), 6.87(m, 2 H), 7.08(m, 1 H), 7.20(s,1 H), 7.30-7.46(m, 5 H)MS ES⁺ m/z 370[MH]⁺ | 45% |
| 20 | 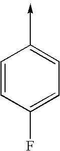 | 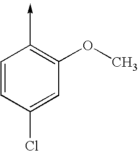 | ¹HNMR(CD₃OD, 400 MHz) δ: 3.20(m,3 H), 3.63(d, 1 H), 3.72(m, 1 H), 3.83(s,3 H), 4.03(m, 2 H), 5.16(d, 1 H), 6.67(m,2 H), 6.97(s, 1 H), 7.06(m, 2 H), 7.40(m,2 H)MS APCl⁺ m/z 352 [MH]⁺Micro analysis found (%); C(54.95),H(5.43), N(3.35); C₁₈H₁₉ClFNO₃.HCl.0.25 H₂Orequires (%); C(55.04),H(5.26), N(3.57) | 54% |
| 21 | 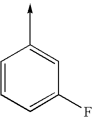 | 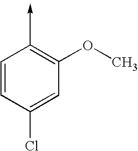 | ¹HNMR(CD₃OD, 400 MHz) δ: 3.14-3.26(m, 3 H), 3.63(d, 1 H), 3.75(m, 1 H),3.87(s, 3 H), 4.01-4.20(m, 2 H), 5.22(d,1 H), 6.72(m, 2 H), 6.98(d, 1 H), 7.05(m,1 H), 7.19(m, 1 H), 7.36(m, 2 H)MS APCl⁺ m/z 352 [MH]⁺ | Quant. |

### EXAMPLES 22 AND 23

The product of example 1 was purified by chiral HPLC on a Chiralpak AS-H™ column, eluting with isopropyl alcohol:hexane:diethylamine, 20:80:0.1. The relevant fraction was evaporated under reduced pressure and the residue was purified by column chromatography on silica gel, eluting with dichloromethane:methanol:0.88 ammonia, 90:10:1. Hydrochloric acid (10 mL in diethyl ether) was added to a solution of the crude compound in dichloromethane and the reaction mixture was concentrated in vacuo. The residue was then azeotroped with diethyl ether to afford compound 22.

Further elution of the chiral HPLC column afforded a second compound that was purified in a similar manner to compound 22, to afford compound 23.

### EXAMPLE 22

### (2S)-2-[(1S)-(4-Chloro-2-methoxyphenoxy)(phenyl)methyl]morpholine hydrochloride

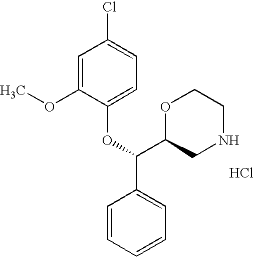

¹HNMR(CD₃OD, 400 MHz) δ: 3.05-3.20 (m, 3H), 3.25 (d, 1H), 3.78-3.87 (m, 4H), 4.08-4.20 (m, 2H), 5.31 (d, 1H), 6.70 (m, 2H), 6.95 (s, 1H), 7.28-7.44 (m, 5H). MS APCI⁺ m/z 334 [MH]⁺. [α]_{D}=+14.4 (c=0.20 in MeOH). Yield: 298 mg (19%) (>99.5% ee by chiral HPLC).

### EXAMPLE 23

### (2R)-2-[(1R)-(4-Chloro-2-methoxyphenoxy)(phenyl)methyl]morpholine hydrochloride

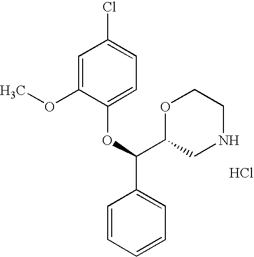

¹HNMR (CD₃OD, 400 MHz) δ: 3.05-3.20 (m, 3H), 3.25 (d, 1H), 3.78-3.87 (m, 4H), 4.08-4.20 (m, 2H), 5.31 (d, 1H), 6.70 (m, 2H), 6.95 (s, 1H), 7.28-7.44 (m, 5H). MS APCI⁺ m/z 334 [MH]⁺. [α]_{D}=−14.8 (c=0.20 in MeOH). Yield: 216 mg (13%) (96.4% ee by chiral HPLC).

### Alternative Method

A solution of the product of preparation 78 (3.37 g, 8.77 mmol) in toluene (20 mL) was added dropwise to an ice cooled solution of Red AI™ (65% wt in toluene, 15 mL) and the mixture was stirred at 5° C. for 1 hour. 2M Sodium hydroxide solution was then carefully added to the reaction mixture, allowing the temperature to rise to 45° C. The mixture was diluted with toluene (50 mL) and the organic phase was separated, washed with 10% potassium carbonate solution and concentrated in vacuo. The residue was purified by column chromatography on silica gel, eluting with ethyl acetate:methanol:0.88 ammonia, 100:0:0 to 90:10:1, followed by dichloromethane:methanol:0.88 ammonia, 90:10:1, to afford the title compound as a gum 1.86 g (58% yield) (>99.5% ee by chiral HPLC). ¹HNMR (CDCl₃, 400 MHz) δ: 2.54-2.68 (m, 2H), 2.75-2.91 (m, 2H), 3.68 (m, 1H), 3.82 (s, 3H), 3.90-4.01 (m, 2H), 5.05 (d, 1H), 6.65 (m, 2H), 6.78 (s, 1H), 7.24-7.35 (m, 5H). MS APCI⁺ m/z 334 [MH]⁺

### EXAMPLE 24

### 5-Chloro-2-[(1R*)-(2R*)-morpholin-2-yl(phenyl)methoxy]benzonitrile hydrochloride

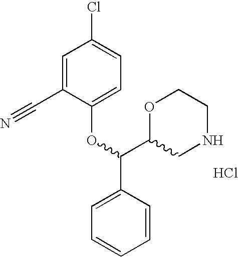

The product of preparation 45 (600 mg, 1.40 mmol) was dissolved in a mixture of trifluoroacetic acid (8 mL) and dichloromethane (4 mL) and the mixture was stirred at room temperature for 4 hours. The reaction mixture was then evaporated under reduced pressure and the residue was dissolved in dichloromethane, washed with sodium hydrogen carbonate solution (×2) and concentrated in vacuo to give a colourless oil. This oil was purified by column chromatography on silica gel, eluting with dichloromethane:methanol:0.88 ammonia, 100:0:0 to 90:10:1. The relevant fractions were evaporated under reduced pressure and the residue was dissolved in dichloromethane. 1M Hydrochloric acid (10 mL in diethyl ether) was added and the solution was concentrated in vacuo to afford the title compound as a white solid in 42% yield.

¹HNMR (CD₃OD, 400 MHz) δ: 3.07-3.20 (m, 3H), 3.29 (m, 1H), 3.88 (m, 1H), 4.17 (m, 1H), 4.25 (m, 1H), 5.58 (d, 1H), 7.01 (d, 1H), 7.30-7.53 (m, 6H), 7.63 (s, 1H). MS ES⁺ m/z 329 [MH]⁺

### EXAMPLES 25 TO 31

The following compounds of the general formula shown below were prepared from the appropriate BOC protected starting material, using a similar method to example 24. Table 9 contains compounds that display (1R*,2R*) relative stereochemistry and Table 10 contains compounds that display (1R*,2S*) relative stereochemistry.

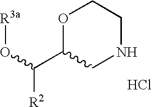

TABLE 9
(1R^{*}, 2R^{*})

| No | R² | R^{3a} | Data | Yield |
|---|---|---|---|---|
| 25 | 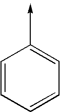 | 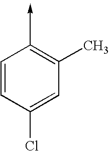 | ¹HNMR(CD₃OD, 400 MHz) δ: 2.30(s,3 H), 2.98-3.19(m, 3 H), 3.21-3.37(m, 1 H),3.88(m, 1 H), 4.09-4.21(m, 2 H), 5.35(d,1 H), 6.65(d, 1 H), 6.91(d, 1 H), 7.11(s,1 H), 7.30-7.42(m, 5 H)MS ES⁺ m/z 318 [MH]⁺ | 34% |
| 26 | 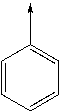 | 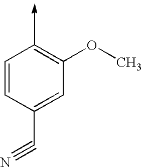 | ¹HNMR(CD₃OD, 400 MHz) δ: 3.01-3.19(m, 2 H), 3.22-3.32(m, 2 H), 3.79-3.90(m, 4 H), 4.18(m, 1 H), 4.21(m, 1 H),5.45(d, 1 H), 6.90(d, 1 H), 7.12(m, 1 H),7.27(m, 1 H), 7.28-7.42(m, 5 H)MS APCl⁺ m/z 325 [MH]⁺ | 96% |
| 27 | 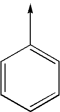 | 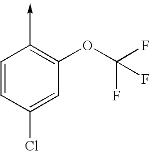 | ¹HNMR(CD₃OD, 400 MHz) δ: 2.77(m,1 H), 2.89(m, 1 H), 3.08(m, 1 H), 3.23(m,1 H), 3.82(m, 1 H), 4.07(m, 1 H), 4.32(m,1 H), 5.34(d, 1 H), 7.30(m, 1 H), 7.39-7.52(m, 7 H)MS APCl⁺ m/z 388 [MH]⁺ | 47% |
| 28 | 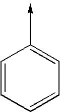 | 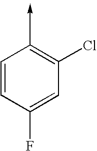 | ¹HNMR(CD₃OD, 400 MHz) δ: 3.01-3.20(m, 4 H), 3.83(m, 1 H), 4.18(m, 2 H),5.45(d, 1 H), 6.89(m, 2 H), 7.19(m, 1 H),7.30-7.45(m, 5 H)MS APCl⁺ m/z 322 [MH]⁺ | 60% |
| 29 | 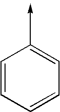 | 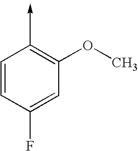 | ¹HNMR(CD₃OD, 400 MHz) δ: 3.09-3.21(m, 4 H), 3.78-3.88(m, 4 H), 4.14(m,2 H), 5.25(d, 1 H), 6.42(m, 1 H), 6.75(m,2 H), 7.30-7.42(m, 5 H)MS APCl⁺ m/z 318 [MH]⁺Micro analysis found (%); C(61.03),H(6.03), N(3.90); C₁₈H₂₀FNO₃.HClrequires (%); C(61.10), H(5.98), N(3.96) | 82% |
| 30 | 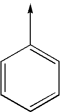 | 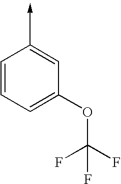 | ¹HNMR(CDCl₃, 400 MHz) δ: 3.01-3.20(m,3 H), 3.30(m, 1 H), 3.83(m, 1 H), 4.12(m,2 H), 5.40(d, 1 H), 6.79(m, 2 H), 6.88(d,1 H), 7.25(m, 1 H), 7.30-7.45(m, 5 H)MS APCl⁺ m/z 354 [MH]⁺Micro analysis found (%); C(55.36),H(5.08), N(3.53); C₁₈H₁₈F₃NO₃.HClrequires(%); C(55.46), H(4.91), N(3.59)MS APCl⁺ m/z 354 [MH]⁺ | Quant. |

Examples 29 and 30: Free base was purified (column chromatography on silica gel, eluting with dichloromethane:methanol:0.88 ammonia, 95:5:0.5) before preparing hydrochloride salt.

TABLE 10
(1R^{*}, 2S^{*})

| No | R¹ | R³ | Data | Yield |
|---|---|---|---|---|
| 31 | 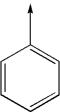 | 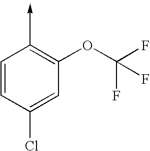 | ¹HNMR(CDCl₃, 400 MHz) δ: 3.29(m, 3 H),3.48(d, 1 H), 3.79(m, 1 H), 4.10(m, 2 H),5.45(d, 1 H), 6.94(d, 1 H), 7.18(d, 1 H),7.30-7.45(m, 6 H)MS APCl⁺ m/z 488 [MH]⁺ | Quant. |

### EXAMPLE 32

### (2R*)-2-[(1R*)-(4-Chloro-2-ethoxyphenoxy)(phenyl)methyl]morpholine hydrochloride

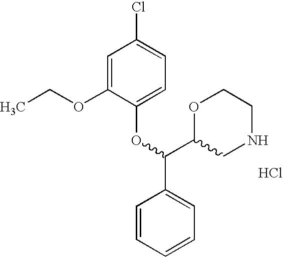

Chloroethyl chloroformate (0.20 mL, 1.85 mmol) was added to a solution of the product of preparation 68 (400 mg, 0.92 mmol) and Proton Sponge® (198 mg, 0.92 mmol) in dichloromethane (20 mL), and the mixture was stirred at room temperature for 18 hours. The mixture was then diluted with dichloromethane and washed with 5% citric acid. The aqueous layer was separated and re-extracted with dichloromethane and the combined organic extracts were dried over sodium sulfate and evaporated under reduced pressure. The residue was purified by column chromatography on silica gel, eluting with dichloromethane:methanol:0.88 ammonia, 95:5:0.5 to 90:10:1. The relevant fractions were concentrated in vacuo and the residue was dissolved in methanol (5 mL). Hydrochloric acid (1 M in diethyl ether) was added and the solvent was evaporated under reduced pressure. The residue was then azeotroped with dichloromethane (×3), diethyl ether (×3) and di-isopropyl ether to afford the title compound as a white solid in 50% yield, 178 mg. ¹HNMR (CDCl₃, 400 MHz) δ: 1.43(t, 3H), 3.02-3.27 (m, 4H), 3.81 (m, 1H), 4.08 (q, 2H), 4.18 (m, 2H), 5.30 (d, 1H), 6.69 (m, 1H), 6.75 (d, 1H), 6.95 (m, 1H), 7.28-7.45 (m, 5H). MS APCI⁺ m/z 348 [MH]⁺. Micro analysis found (%); C(59.25), H(6.29), N(3.53); C₁₉H₂₂ClNO₃.HCl. requires (%); C(59.38), H(6.03), N(3.64).

### EXAMPLE 33

### (2S*)-2-[(1R*)-(4-chloro-2-ethoxyphenoxy)(Phenyl)methyl]morpholine hydrochloride

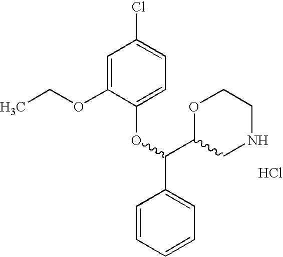

Chloroethyl chloroformate (0.25 mL, 2.28 mmol) was added to a solution of the product of preparation 71 (500 mg, 1.14 mmol) and Proton Sponge® (245 mg, 1.14 mmol) in dichloromethane (20 mL), and the mixture was stirred at room temperature for 18 hours. The mixture was then diluted with dichloromethane and washed with 5% citric acid. The aqueous layer was separated, extracted with dichloromethane and the combined organic solutions were dried over sodium sulfate and evaporated under reduced pressure. The residue was then dissolved in methanol and heated under reflux for 3 hours. The solvent was evaporated under reduced pressure and the residue was taken up in 1 M sodium hydroxide solution and extracted with dichloromethane. The aqueous layer was separated and re-extracted with dichloromethane and the combined organic extracts were dried over sodium sulfate and concentrated in vacuo. The residue was then purified by column chromatography on silica gel, eluting with dichloromethane:methanol:0.88 ammonia, 95:5:0.5 to 90:10:1. The relevant fractions were concentrated in vacuo and the residue was dissolved in methanol (5 mL). Hydrochloric acid (1 M in diethyl ether) was added and the solvent was evaporated under reduced pressure. The residue was then azeotroped with dichloromethane (×3), diethyl ether (×3) and di-isopropyl ether to afford the title compound as a white solid in 54% yield, 214 mg. ¹HNMR (CD₃OD, 400 MHz) δ: 1.45(t, 3H), 3.10-3.28 (m, 3H), 3.64 (m, 1H), 3.76 (m, 1H), 4.06 (m, 4H), 5.19 (d, 1H), 6.67 (m, 2H), 6.93 (s, 1H), 7.22-7.42 (m, 5H). MS APCI⁺ m/z 348 [MH]⁺ Micro analysis found (%); C(59.38), H(6.13), N(3.55); C₁₉H₂₂ClNO₃.HCl. requires (%); C(59.38), H(6.03), N(3.64).

### EXAMPLES 34 TO 36

The following compounds of general formula shown below were prepared from the appropriate benzyl protected starting material, using a similar method to example 33. All compounds display (1R*,2S*) relative stereochemistry and are represented by Table 11.

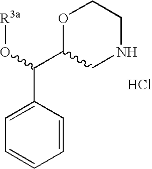

TABLE 11
(1R^{*}, 2S^{*})

| No | R^{3a} | Data | Yield |
|---|---|---|---|
| 34 | 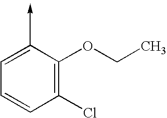 | ¹HNMR(CD₃OD, 400 MHz) δ: 1.45(t, 3 H),3.16(m, 1 H), 3.30(m, 1 H), 3.49(m, 2 H),3.82(m, 1 H), 4.04-4.20(m, 4 H), 5.19(d,1 H), 6.73(d, 1 H), 6.82(m, 1 H),6.96(s, 1 H), 7.30-7.50(m, 5 H)MS APCl⁺ m/z 348 [MH]⁺Micro analysis found (%); C(59.12),H(6.03), N(3.64); C₁₉H₂₂ClNO₃.HCl.requires (%); C(59.38), H(6.03), N(3.64) | Quant. |
| 35 | 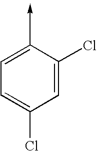 | ¹HNMR(CD₃OD, 400 MHz) δ:3.20(m, 1 H), 3.30(m, 2 H), 3.62(d, 1 H),3.80(m, 1 H), 4.10(m, 2 H), 5.48(d, 1 H),6.85(d, 1 H), 7.10(d, 1 H), 7.30-7.42(m,6 H)MS ES⁺ m/z 338 [MH]⁺ | 69% |
| 36 | 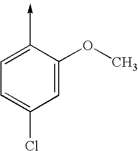 | ¹HNMR(CD₃OD, 400 MHz) δ:3.00-3.32(m, 3 H), 3.64(d, 1 H), 3.84(s,3 H), 4.00(m, 2 H), 4.25(m, 1 H), 5.07(d,1 H), 6.50(d, 1 H), 6.63(d, 1 H), 6.80(s,1 H), 7.21-7.39(m, 5 H), 10.03(brs, 2 H)MS ES⁺ m/z 334 [MH]⁺ | 34% |

### EXAMPLE 37

The NRI Kᵢ; and SRI Kᵢ; values of the compounds of Examples 1-36 were determined as follows. All of the compounds exhibited a Ki value less than 200 nM at the serotonin transporter and a Ki value less than 200 nM at the noradrenaline transporter.

### Biological Activity

The compounds were tested for biological activity by their ability to compete with and inhibit the binding of [³H]Nisoxetine to the human noradrenaline transporter, [³H]Citalopram to the human serotonin transporter and [³H]WIN-35428 to the human dopamine transporter as follows.

### (i) Membrane Preparation

Human embryonic kidney cells (HEK-293) stably transfected with either the human serotonin transporter (hSERT), noradrenaline transporter (hNET) or dopamine transporter (hDAT) were cultured under standard cell culture techniques (cells were grown at 37° C. and 5% CO₂ in either Dulbecco's Modified Eagle's Medium (DMEM) culture media supplemented with 10% dialysed foetal calf serum (FCS), 2 mM L-glutamine and 250 μg/ml geneticin (hSERT and hNET cells) or DMEM-culture media supplemented with 5% FCS, 5% new-born calf serum, 2 mM L-glutamine and 2.5 mg/ml puromycin (hDAT cells)). Cells were harvested, pelleted by centrifugation and re-suspended in ice-cold membrane prep buffer. The cell suspension was then homogenized, large particulate matter removed by low speed centrifugation and the supernatant re-centrifuged (35,000×g, 30 minutes at 4° C.). The pelleted membranes were re-suspended in membrane prep buffer, protein concentrations measured (Sigma protein kit) and the membrane suspension stored frozen in aliquots.

### (i) Determination of Inhibitor Potency

Prior to assay, membranes containing the respective human transporter protein were pre-coupled to the appropriate scintillation-proximity assay (SPA) bead, i.e., PVT WGA SPA beads (Amersham) for hNET and hDAT and YSi WGA SPA beads (Amersham) for hSERT, so as to minimise ligand depletion and maximise the assay window for the corresponding [³H] ligand. SPA beads re-suspended (−50 mg/ml) in assay buffer (1.5×) were pre-coupled with membranes (typically 5-40 μg membrane per mg of bead) by incubating with gentle shaking for 2 hours at 4° C. After coupling, the beads/membranes were collected by centrifugation and washed and re-suspended in assay buffer (1.5×) with gentle stirring at the required concentration for the assay (typically 5-40 mg beads/ml).

Also prior to assay, each [³H] ligand was diluted in assay buffer (1.5×) to give a stock concentration of 3× the final assay concentration (typical final concentrations=12 nM [³H]Nisoxetine (Amersham), 2.5 nM [³H]Citalopram (Amersham) and 10 nM [³H]WIN-35428 (Perkin Elmer), which were confirmed by scintillation counting). Finally, all test compounds were dissolved in 100% DMSO at 4 mM and diluted down in 1% DMSO in water to give appropriate test concentrations.

Assays were carried out in 384-well NBS plates (Costar). For each assay, 20 μl of the appropriate dilution of either test compound, a standard inhibitor (positive control) or compound vehicle (DMSO in water; final DMSO concentration was 0.25% in each assay well) was added to 20 μl of the appropriate stock of [³H] ligand. 20 μl of the corresponding bead/membrane preparation was then added and the plate sealed prior to incubation with shaking for 1 hour. The assay plates were then incubated at room temperature for at least a further 6 hours (to attain equilibrium) with dark adaptation, before direct scintillation counting.

Potency of test compounds was quantified as IC₅₀ values (concentration of test compound required to inhibit the specific binding of radio-labelled ligand to the respective transporter protein by 50% relative to maximum (compound vehicle only) and minimum (complete inhibition by standard inhibitor) responses). The Ki value was derived for each compound by conversion of the IC₅₀ value using the Cheng-Prusoff equation and the experimentally measured free ligand concentration and Kd for the batch of membrane used in assay (typical Kd values: ˜30 nM Nisoxetine, ˜8 nM Citalopram and ˜15 nM WIN-35428).

(iii) Membrane Prep Buffer

HEPES (20 mM) HEPES

1 complete protease inhibitor tablet (Roche)/50 ml

pH 7.4 at room temperature, store at 4° C.

Assay Buffer (1.5× assay concentration)

HEPES (30 mM)

NaCl (180 mM)

pH 7.4 at room temperature, store at 4° C.

### (iv) Summary of Assay Parameters

| | hNET assay | hSERT assay | hDAT assay |
|---|---|---|---|
| Transporter | hNET/PVT | hSERT/YSi | hDAT/PVT WGA |
| membrane/SPA | WGA | WGA | |
| bead type | | | |
| Ligand/ | ³H-Nisoxetine | ³H-citalopram | ³H-WIN-35428 |
| concentration | (12 nM) | (2.5 nM) | (10 nM) |
| Incubation time | 7 | 7 | 7 |
| (hrs) | | | |

### EXAMPLE 37

### (2S)-2-[(1S)-(2-chloro-4-fluorophenoxy)-(3-fluorophenyl)methyl]morpholine hydrochloride

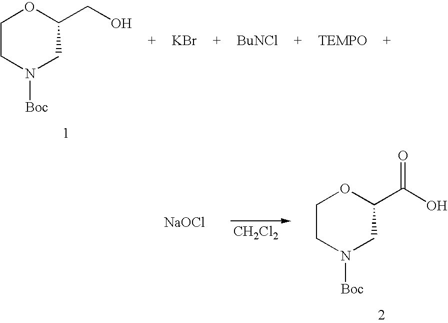

A 500 ml flask was charged with 5.0 g (23 mmol) of (S)-2-hydroxymethyl-morpholine-4-carboxylic acid tert-butyl ester (1) (Beard Research), 0.219 g KBr (1.84 mmol), 0.3518 g (1.27 mmol) Bu₄NCl, 54 mg (0.35 mmol) TEMPO (2,2,6,6-tetramethyl-1-piperidinyloxy free radical), 150 ml dichloromethane, and 50 ml 1M sodium bicarbonate solution. The biphasic solution was stirred and cooled in a 0° C. bath. To this biphasic solution was added dropwise a mixture of 50 ml 10% sodium hypochlorite, 50 ml saturated NaCl solution, and 25 ml 1 M sodium bicarbonate over about 45 minutes. The solution was stirred overnight. The layers were separated, and the aqueous layer was washed with dichloromethane. The aqueous layer was then acidified (with concentrated HCl) slowly to a pH of 2. The aqueous layer was extracted twice with dichloromethane, and the combined organic layers were dried with sodium sulfate. The drying agent was removed by filtration, and the solvent was removed under reduced pressure yielding 1.60 g (6.92 mmol) of (S)-morpholine-2,4-dicarboxylic acid 4-tert-butyl ester 2 as a white/yellow solid. The acid was carried on with no further purification.

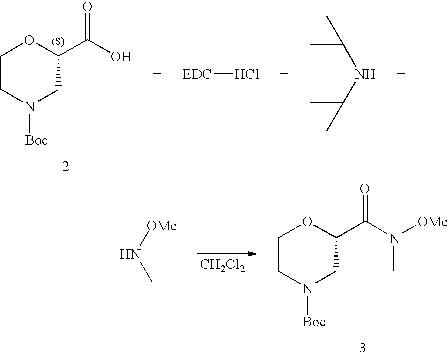

1.60 g (6.92 mmol) of (S)-morpholine-2,4-dicarboxylic acid 4-tert-butyl ester was placed in a 100 ml flask. To this flask was added 30 ml dry dichloromethane, 1.18 ml (6.78 mmol) diisopropylethylamine, 662 mg (6.78 mmol) N,O-dimethylhydroxylamine hydrochloride, and 1.37 g (7.12 mmol) 1-[3-(dimethylamino)propyl]-3-ethylcarbodiimide hydrochloride (EDC-HCl). The mixture was stirred for 5 hours. The reaction mixture was diluted with dichloromethane, washed three times with water, once with saturated aqueous NH₄Cl, and then once with brine. The dichloromethane layers were dried over sodium sulfate. The drying agent was removed by filtration, and the solvent was removed under reduced pressure. The crude material was purified by column chromatography using 1:1 hexanes/ethyl acetate as the eluent. This procedure provided 1.08 g (3.94 mmol) (S)-2-(methoxy-methyl-carbamoyl)-morpholine-4-carboxylic acid tert-butyl ester 3 as a clear oil.

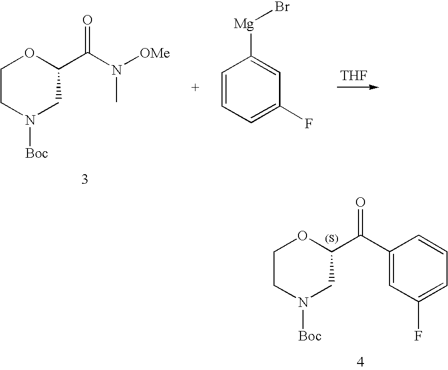

A 250 ml flask was charged with 4.14 g (15.1 mmol) of (S)-2-(methoxy-methyl-carbamoyl)-morpholine-4-carboxylic acid tert-butyl ester and 40 ml dry THF (tetrahydrofuran). The mixture was cooled to −78° C., and 30 ml 1 M (30 mmol) 3-fluorophenylmagnesium bromide was added slowly. The mixture was allowed to stir at −78° C. for 30 min, and then was transferred to a −20° C. bath and stirred for 1 hour. The reaction mixture was cooled back to −78° C. and quenched with saturated aqueous NH₄Cl. The reaction mixture was then allowed to warm to 20° C., and the THF was removed under reduced pressure. The resulting crude material was partitioned between water and dichloromethane. The dichloromethane layer was collected and the aqueous layer was extracted washed three times with dichloromethane. The combined dichloromethane layers were dried over MgSO₄. The drying agent was removed by filtration, and the solvent was removed under reduced pressure. The resulting crude material was purified by column chromatography using 3:1 hexanes/ethyl acetate as the eluent. This procedure provided 3.83 g (12.4 mmol) of (S)-2-(3-fluoro-benzoyl)-morpholine-4-carboxylic acid tert-butyl ester 4 as a white solid.

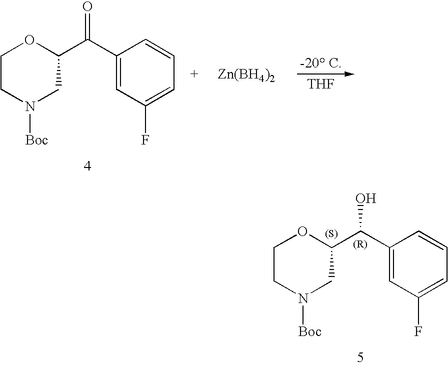

A 250 ml flask was charged with 2.13 g (6.89 mmol) of the ketone 4 and 40 ml dry THF. The resulting solution was cooled in a −20° C. bath. Slowly 15 ml of 0.5 M (7.5 mmol) zinc borohydride (Zn(BH₄)₂)solution in THF was added, and the resulting mixture was stirred for 1 hour. The reaction was quenched by the addition of saturated aqueous ammonium chloride. The reaction mixture was allowed to warm to 20° C., and the THF was removed under reduced pressure. The resulting crude material was partitioned between water and dichloromethane. The dichloromethane layer was collected, and the aqueous layer was extracted twice with dichloromethane. The combined dichloromethane layers were dried with MgSO₄. The drying agent was removed by filtration, and the dichloromethane was removed under reduced pressure. The resulting crude oil was purified by column chromatography using a 4:1 mixture of 25% dichloromethane-hexanes/ethyl acetate. This procedure provided 1.46 g (1.66 mmol) of (2S)-2-[(1R)-(3-fluoro-phenyl)-hydroxy-methyl]-morpholine-4-carboxylic acid tert-butyl ester 5 as a white solid.

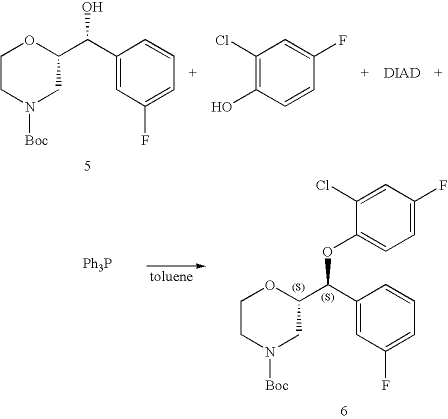

A 50 ml flask was charged with 400 mg (1.29 mmol) (2S)-2-[(1R)-(3-fluoro-phenyl)-hydroxy-methyl]-morpholine-4-carboxylic acid tert-butyl ester 5 and 15 ml toluene. To the solution was added 543 μl (5.14 mmol) 2-chloro-4-fluorophenol and 876 mg (3.34 mmol) triphenyl phosphine. This mixture was cooled in a 0° C. bath, and 622 μl (3.21 mmol) diisopropyl azodicarboxylate (DIAD) was added slowly. The mixture was allowed to warm slowly to room temperature overnight (by the melting of the ice). The mixture was stirred until the chiral alcohol 5 was no longer detectable by thin layer chromatography. The toluene was removed under reduced pressure, and the resulting crude oil was purified directly using column chromatography and 9:1 Hexanes/Ethyl Acetate as the eluent. This procedure provided 351 mg of 6 (2S)-2-[(1S)-(2-chloro-4-fluorophenoxy)-(3-fluorophenyl)methyl]morpholine-4-carboxylic acid tert-butyl ester) as a foam.

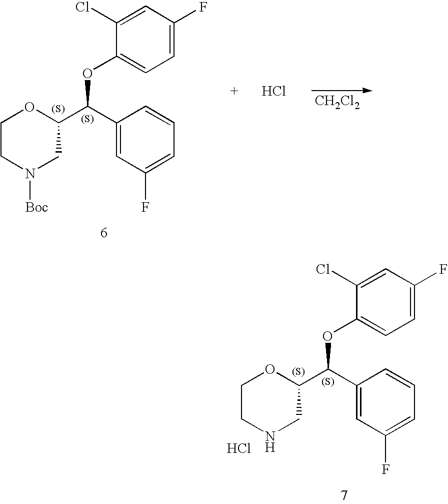

A 50 ml flask containing 340 mg (0.77 mmol) of (2S)-2-[(1S)-(2-chloro-4-fluorophenoxy)-(3-fluorophenyl)methyl]morpholine-4-carboxylic acid tert-butyl ester (6) was charged with 15 ml dichloromethane and 1.55 ml (3.1 mmol) 2 M HCl in diethyl ether. The flask was capped and stirred overnight. The solvent was then removed under reduced pressure leaving 308 mg (0.82 mmol) of the hydrochloride salt 7 ((2S)-2-[(1S)-(2-chloro-4-fluorophenoxy)-(3-fluorophenyl)methyl]morpholine hydrochloride) as a yellowish solid.

### EXAMPLES 38-79

The compounds of Examples 38-79 were made in a manner analogous to the synthesis of the compound of Example 37.

| Ex. | |
|---|---|
| No. | Compound |
| 38 | (2S)-2-[(1S)-(4-chloro-2-methoxyphenoxy) (phenyl) methyl] morpholine hydrochloride |
| 39 | (2S)-2-[(1S)-(2,3-Difluorophenoxy)(3-fluorophenyl)methyl] morpholine hydrochloride |
| 40 | (2S)-2-[(1S)-(2-Methoxy-4-methylphenoxy) phenylmethyl] morpholine hydrochloride |
| 41 | (2S)-2-[(1S)-(2-Chloro-5-fluorophenoxy)(3-fluorophenyl)methyl] morpholine hydrochloride |
| 42 | (2S)-2-[(1S)-(2-methoxy-4-methylphenoxy)4-fluorophenyl) methyl] morpholine hydrochloride |
| 43 | (2R)-2-[(1R)-(2-Methoxy-4-methylphenoxy)4-Fluorophenyl) methyl] morpholine hydrochloride |
| 44 | (2S)-2-[(1S)-(4-Chloro-2-fluorophenoxy)(3-fluorophenyl)methyl] morpholine hydrochloride |
| 45 | (2S)-2-[(1S)-(4-Chloro-2-methoxyphenoxy)(3-fluorophenyl)methyl] morpholine hydrochloride |
| 46 | (2S)-2-[(1S)-(4-Fluoro-2-methoxyphenoxy)(3-fluorophenyl)methyl] morpholine hydrochloride |
| 47 | (2S)-2-[(1S)-(2,6-Difluorophenoxy)-(3-fluorophenyl)methyl] morpholine hydrochloride |
| 48 | (2S)-2-[(1S)-(2-Chloro-3,5-difluorophenoxy)(3-fluorophenyl)methyl] morpholine hydrochloride |
| 49 | (2S)-2-[(1S)-(3-Fluorophenyl)-o-tolyloxy-methyl] morpholine hydrochloride |
| 50 | (2S)-2-[(1S)-(2-Fluoro-6-methoxyphenoxy)(3-fluorophenyl)methyl] morpholine hydrochloride |
| 51 | (2S)-2-[(1S)-(3-Fluorophenyl)-(2-methoxy-5-methylphenoxy)methyl] morpholine hydrochloride |
| 52 | (2S)-2-[(1S)-(3-Chlorophenyl) (4-fluoro-2-methoxyphenoxy) methyl] morpholine hydrochloride |
| 53 | (2S)-2-[(1S)-(2-Chloro-5-fluorophenoxy) (3-chlorophenyl) methyl] morpholine hydrochloride |
| 54 | (2S)-2-[(1S)-(4-Chloro-2-methoxy phenoxy)-m-tolyl-methyl] morpholine hydrochloride |
| 55 | (2S)-2-[(1S)-(2-Methoxy-4-methyl phenoxy)-m-tolyl-methyl] morpholine hydrochloride |
| 56 | (2S)-2-[(1S)-(2-Chloro-4-fluorophenoxy)-m-tolyl-methyl] morpholine hydrochloride |
| 57 | (2S)-2-[(1S)-(4-Fluoro-2-methoxy phenoxy)-m-tolyl-methyl] morpholine hydrochloride |
| 58 | (2S)-2-[(1S)-(2,4-Dimethoxy phenoxy)-m-tolyl-methyl] morpholine hydrochloride |
| 59 | (2S)-2-[(1S)-(2-Chloro-5-fluorophenoxy)-m-tolyl-methyl] morpholine hydrochloride |
| 60 | (2S)-2-[(1S)-(2-Chloro-6-fluorophenoxy) (3-fluorophenyl) methyl] morpholine hydrochloride |
| 61 | (2S)-2-[(1S)-(4-Chloro-2-methoxyphenoxy) (3-methoxyphenyl) methyl] morpholine |
| | hydrochloride |
| 62 | (2S)-2-[(1S)-(2-Methoxy-4-methylphenoxy) (3-methoxyphenyl) methyl] morpholine |
| | hydrochloride |
| 63 | (2S)-2-[(1S)-(2-Chloro-4-fluorophenoxy)-(3-methoxyphenyl) methyl] morpholine hydrochloride |
| 64 | (2S)-2-[(1S)-(2,4-Difluorophenoxy) (3-fluoro-phenyl) methyl] morpholine hydrochloride |
| 65 | (2S)-2-[(1S)-(3-Fluorophenyl) (2,4,6-trifluorophenoxy) methyl] morpholine hydrochloride |
| 66 | (2S)-2-[(1S)-(3-Fluorophenyl) (2-propylphenoxy) methyl] morpholine hydrochloride |
| 67 | (2S)-2-[(1S)-(3-Fluorophenyl) (4-trifluoromethyl phenoxy) methyl] morpholine hydrochloride |
| 68 | (2S)-2-[(1S)-(4-Fluoro-2-methoxyphenoxy) (3-methoxyphenyl) methyl] morpholine |
| | hydrochloride |
| 69 | (2S)-2-[(1S)-(2-Chloro-5-fluorophenoxy) (3-methoxyphenyl) methyl] morpholine hydrochloride |
| 70 | (2S)-2-[(1S)-(2-Bromo-4-fluorophenoxy) (3-methoxyphenyl) methyl] morpholine hydrochloride |
| 71 | (2S)-2-[(1S)-(4-Chloro-phenyl) (4-fluoro-2-methoxyphenoxy) methyl] morpholine hydrochloride |
| 72 | (2S)-2-[(1S)-(2-Chloro-4-fluorophenoxy) (4-chlorophenyl) methyl] morpholine hydrochloride |
| 73 | (2S)-2-[(1S)-(4-Chloro-2-methoxyphenoxy) (4-chlorophenyl) methyl] morpholine hydrochloride |
| 74 | (2S)-2-[(1S)-(2-Chloro-4-fluorophenoxy) (4-fluorophenyl) methyl] morpholine hydrochloride |
| 75 | (2S)-2-[(1S)-(4-Chlorophenyl)-(2-methoxy-4-methylphenoxy) methyl] morpholine hydrochloride |
| 76 | (2S)-2-[(1S)-(4-Chloro-2-fluorophenoxy) (4-chlorophenyl) methyl] morpholine hydrochloride |
| 77 | (2S)-2-[(1S)-(2-Bromo-4-chlorophenoxy) (4-chlorophenyl) methyl] morpholine hydrochloride |
| 78 | 2-[(1S)-(3-Fluorophenyl) [(2S)-morpholin-2-yl] methoxy] benzonitrile hydrochloride |
| 79 | (2S)-2-[(1S)-(3-Fluorophenyl)(2-methoxy-4-methylphenoxy)-methyl]morpholine hydrochloride |

### EXAMPLE 80

### (2S)-2-[(1S)-(3-chloro-2-fluoro-phenoxy)-phenyl-methyl]-morpholine fumarate salt

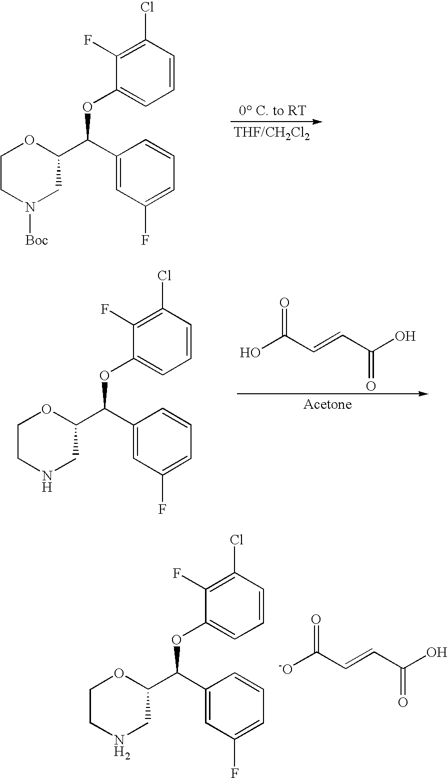

The (2S)-2-[(1S)-(3-chloro-2-fluoro-phenoxy)-phenyl-methyl]-morpholine-4-carboxylic acid tert-butyl ester was prepared in a manner analogous to that used in the preparation of (2S)-2-[(1S)-(2-chloro-4-fluorophenoxy)-(3-fluorophenyl)methyl]morpholine-4-carboxylic acid tert-butyl ester in the synthesis of the compound of Example 37. (2S)-2-[(1S)-(3-Chloro-2-fluoro-phenoxy)-phenyl-methyl]-morpholine-4-carboxylic acid tert-butyl ester (0.54 g, 1.28 mmol) was taken up in 10 ml dichloromethane, cooled to 0° C., and 4 ml trifluoroacetic acid (TFA) was added. The ice bath was removed, and the reaction mixture was stirred at room temperature for 1 hour. The solvent and acid were removed under reduced pressure. To the residual oil was added 15 ml H₂O and 15 ml CH₂Cl₂. The biphasic mixture was shaken, and the aqueous layer collected. The pH value of the mixture was adjusted to 13 by adding 1.0 M NaOH solution. The aqueous phase was extracted using 15 ml CH₂Cl₂. The organic phase was washed with 20 ml H₂O and dried over Na₂SO₄. The solvent was removed under reduced pressure providing 0.41 g (1.24 mmol) (2S)-2-[(1S)-(3-chloro-2-fluoro-phenoxy)-phenyl-methyl]-morpholine as an oil. The (2S)-2-[(1S)-(3-chloro-2-fluoro-phenoxy)-phenyl-methyl]-morpholine was then dissolved in 5 ml acetone. The resulting solution was added to a solution of 144 mg (1.24 mmol) fumaric acid in 30 ml acetone and stirred at room temperature. A white precipitate gradually appeared. The precipitate was collected by filtration, washed by four times with 5 ml of acetone, and dried under vacuum for at least 24 hours to give 0.46 g (1.05 mmol) of (2S)-2-[(1S)-(3-chloro-2-fluoro-phenoxy)-phenyl-methyl]-morpholine fumarate salt.

### EXAMPLES 81-102

The compounds of Examples 81-102 were made in a manner analogous to the synthesis of the compound of Example 80.

| Ex. | |
|---|---|
| No. | Compound |
| 81 | (2S)-2-[(1S)-(2,3-Dichlorophenoxy)phenylmethyl] morpholine fumarate |
| 82 | (2S)-2-[(1S)-(3-Chloro-2-methylphenoxy)phenylmethyl] morpholine fumarate |
| 83 | (2S)-2-[(1S)-(2-Chloro-3,5-difluorophenoxy)phenyl methyl]morpholine fumarate |
| 84 | (2S)-2-[(1S)-(5-Chloro-2-methoxyphenoxy)phenylmethyl]morpholine fumarate |
| 85 | (2S)-2-[(1S)-(Pentafluorophenyloxy) (phenyl) methyl] morpholine fumarate |
| 86 | (2S)-2-[(1S)-Phenyl-(2,4,6-trifluorophenoxy) methyl] morpholine fumarate |
| 87 | (2S)-2-[(1S)-(2-Chloro-5-methylphenoxy) phenyl methyl] morpholine fumarate |
| 88 | (2S)-2-[(1S)-(2-Chloro-5-trifluoromethyl phenoxy) phenyl methyl] morpholine fumarate |
| 89 | (2S)-2-[(1S)-(2,5-Dichloro phenoxy) phenyl methyl] morpholine fumarate |
| 90 | (2S)-2-[(1S)-(3-Chloro-2-fluorophenoxy) phenyl methyl] morpholine fumarate |
| 91 | (2S)-2-[(1S)-Phenyl-(3,4,6-trichloro-2-methoxyphenoxy) methyl] morpholine fumarate |
| 92 | (2S)-2-[(1S)-(3-Chloro-2-methoxy phenoxy) phenyl methyl] morpholine fumarate |
| 93 | (2S)-2-[(1S)-(4,5-Dichloro-2-methoxy phenoxy) phenyl methyl] morpholine fumarate |
| 94 | (2S)-2-[(1S)-(4-Bromo-2-methoxy phenoxy) phenyl methyl] morpholine fumarate |
| 95 | (2S)-2-[(1S)-Pentachlorophenyloxy phenyl methyl] morpholine fumarate |
| 96 | (2S)-2-[(1S)-(2-Chloro-4-methoxy phenoxy) phenyl methyl] morpholine fumarate |
| 97 | (2S)-2-[(1S)-(2-Chloro-5-methoxy phenoxy) phenyl methyl] morpholine fumarate |
| 98 | (2S)-2-[(1S)-Phenyl-(2,4,6-trichlorophenoxy) methyl] morpholine fumarate |
| 99 | (2S)-2-[(1S)-(2-Methoxy-4-trifluoromethyl phenoxy) phenyl methyl] morpholine fumarate |
| 100 | (2S)-2-[(1S)-(4-Chloro-2-methoxy phenoxy) (3-chloro phenyl) methyl] morpholine fumarate |
| 101 | (2S)-2-[(1S)-(3-Chlorophenyl) (2-methoxy-4-methyl phenoxy) methyl] morpholine fumarate |
| 102 | (2S)-2-[(1S)-(2-Chloro-4-fluorophenoxy) (3-chlorophenyl) methyl] morpholine fumarate |

### EXAMPLE 103

### (2S)-2-[(1S)-(4-chloro-2-methoxyphenoxy)(pyridin-2-yl)methyl]morpholine fumarate

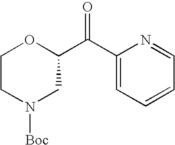

To a solution of 2-iodopyridine (6.73 g, 32.8 mmol) in THF (tetrahydrofuran) (150 ml) was added a 2.0M solution of ethylmagnesium chloride in THF (15.9 ml, 31.9 mmol) over 15 minutes. The solution was stirred at room temperature for 30 minutes. This mixture was added dropwise over 60 minutes to a cold (−40° C. bath) solution of tert-butyl (2S)-2-{[methoxy(methyl)amino]carbonyl}morpholine-4-carboxylate in THF (100 ml). The mixture was stirred an additional 30 minutes at −40° C. Saturated aqueous NH₄Cl (150 ml) was added to the cold solution, the cold bath removed and the reaction warmed to room temperature. The layers were separated and the organic layer was washed with saturated aqueous NaHCO₃ (100 ml). The organic layer was dried over Na₂SO₄, filtered and concentrated under reduced pressure. The residue was purified by silica gel chromatography eluting with 20-50% EtOAc in hexanes to provide tert-butyl (2S)-2-(pyridin-2-ylcarbonyl)morpholine-4-carboxylate as a white solid (4.38 g). ¹H NMR (400 MHz, CHLOROFORM-D) 8 ppm 1.4 (s, 9H) 2.9 (bs, 1H) 3.1 (ddd, J=13.4, 10.9, 3.5 Hz, 1H) 3.7 (td, J=11.2, 2.9 Hz, 1H) 3.9 (d, J=11.4 Hz, 1H) 4.1 (d, J=11.3 Hz, 1H) 4.5 (d, J=12.6 Hz, 1H) 5.4 (d, J=7.7 Hz, 1H) 7.5 (ddd, J=7.6, 4.8, 1.1 Hz, 1H) 7.9 (td, J=7.7, 1.5 Hz, 1H) 8.1 (d, J=7.9 Hz, 1H) 8.7 (d, J=4.1 Hz, 1H). MS (APCI) 293.1 (M+1).

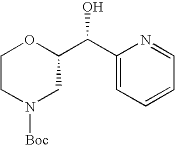

In a glove box, tert-butyl (2S)-2-(pyridin-2-ylcarbonyl)morpholine-4-carboxylate (4.3 g, 15 mmol), K₂CO₃ (0.508 g) and dichloro[(S)-(−)-2,2′-bis(diphenylphosphino)-1,1′-binaphthyl][(2S)-(+)-1,1-bis(4-methoxyphenyl)-3-methyl-1,2-butanediamine]ruthenium (II) (0.033 g) were combined in isopropyl alcohol (IPA) (80 ml) and THF (20 ml). The mixture was stirred under an atmosphere of H₂ (50 psi) for 16 hours then filtered and concentrated under reduced pressure. The residue was purified by silica gel chromatography eluting with 40-75% EtOAc in hexanes to provide tert-butyl (2S)-2-[(1R)-hydroxy(pyridin-2-yl)methyl]morpholine-4-carboxylate as a white solid (4.1 g). ¹H NMR (400 MHz, METHANOL-D4) δ ppm 1.4 (s, 9H) 2.9 (bs, 2H) 3.4 (td, J=11.7, 3.0 Hz, 1H) 3.6 (ddd, J=10.5, 5.9, 2.5 Hz, 1H) 3.8 (m, 2H) 3.9 (dt, J=13.2, 2.1 Hz, 1H) 4.7 (d, J=5.8 Hz, 1H) 7.3 (ddd, J=7.6, 4.9, 1.2 Hz, 1H) 7.5 (d, J=7.9 Hz, 1H) 7.8 (td, J=7.7, 1.8 Hz, 1H) 8.5 (dt, J=4.9, 0.9 Hz, 1H). MS (APCI) 295.1 (M+1).

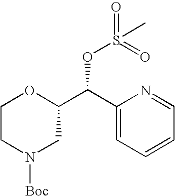

To a cold (−10° C.) solution of triethylamine (2.4 ml, 17.3 mmol) and tert-butyl (2S)-2-[(1R)-hydroxy(pyridin-2-yl)methyl]morpholine-4-carboxylate (4.0 g, 14 mmol) in CH₂Cl₂ (140 ml) was added a solution of methanesulfonyl chloride (1.22 ml, 15.6 mmol) in CH₂Cl₂ (10 ml). The mixture was allowed to warm to room temperature and then stirred until no starting alcohol remained by thin-layer chromatography. Water (100 ml) was added and the mixture was stirred rapidly for 1 minute at which time saturated aqueous NaHCO₃ (5 ml) was added and the mixture stirred for an additional minute. The layers were separated and the aqueous was extracted with CH₂Cl₂ (100 ml). The combined organic layers were washed with brine, dried over Na₂SO₄ and concentrated to an oil which solidified on standing to give tert-butyl (2S)-2-[(1R)-[(methylsulfonyl)oxy](pyridin-2-yl)methyl]morpholine-4-carboxylate (5.0 g). ¹H NMR (400 MHz, METHANOL-D4) δ ppm 1.4 (s, 9H) 3.0 (s, 2H) 3.1 (s, 3H) 3.5 (td, J=11.6, 2.9 Hz, 1H) 3.8 (m, J=13.4, 2.9, 1.4, 1.4 Hz, 1H) 3.9 (m, 1H) 4.0 (m, 1H) 5.6 (d, J=5.0 Hz, 1H) 7.4 (ddd, J=7.6, 4.9, 1.1 Hz, 1H) 7.6 (dt, J=7.9, 1.0 Hz, 1H) 7.9 (td, J=7.8, 1.8 Hz, 1H) 8.6 (ddd, J=4.9, 1.7, 0.9 Hz, 1H). MS (APCI) 373.1 (M+1).

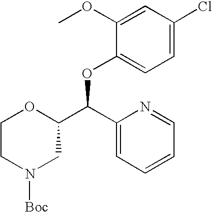

Tert-butyl (2S)-2-[(1R)-[(methylsulfonyl)oxy](pyridin-2-yl)methyl]morpholine-4-carboxylate (0.375 g, 1.0 mmol), 4-chloro-2-methoxyphenol (0.216 g, 1.35 mmol), K₂CO₃ (0.56 g, 4.0 mmol) and tert-butanol (0.10 ml, 1.0 mmol) were combined in toluene (10 ml) and heated to 105° C. After 24 hours, additional 4-chloro-2-methoxyphenol (0.100 g), K₂CO₃ (0.56 g) and tert-butanol (0.20 ml) were added. The mixture was heated for another 24 hr (48 hours total) then cooled to room temperature and filtered. Silica gel chromatography eluting with 15-50% EtOAc in hexanes to provided tert-butyl (2S)-2-[(1S)-(4-chloro-2-methoxyphenoxy)(pyridin-2-yl)methyl]morpholine-4-carboxylate as an oil (0.245 g). ¹H NMR (400 MHz, CHLOROFORM-D) δ ppm 1.4 (s, 9H) 3.0 (t, J=12.4 Hz, 1H) 3.5 (t, J=11.7 Hz, 1H) 3.8 (d, J=15.5 Hz, 5 H) 3.9 (d, J=11.5 Hz, 2H) 5.2 (d, J=4.4 Hz, 1H) 6.6 (d, J=8.7 Hz, 1H) 6.7 (m, 1H) 6.8 (s, 1H) 7.2 (m, 1 H) 7.5 (d, J=7.9 Hz, 1H) 7.6 (t, J=7.1 Hz, 1H) 8.6 (d, J=5.0 Hz, 1H). MS (APCI) 435.1 (M+1).

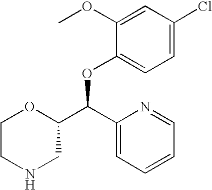

To a solution of tert-butyl (2S)-2-[(1S)-(4-chloro-2-methoxyphenoxy)(pyridin-2-yl)methyl]morpholine-4-carboxylate (0.223 g, 0.51 mmol) in CH₂Cl₂ (5 ml) was added 2.0M HCl in diethyl ether (2.0 ml, 4.0 mmol). The mixture was stirred at room temperature for 18 h then concentrated under reduced pressure. The residue was partitioned between 5% aqueous NaOH (10 ml) and CH₂Cl₂ (50 ml). The organic layer was dried over Na₂SO₄, filtered and concentrated under reduced pressure. The residue was dissolved in IPA (3 ml) and treated with 0.2M fumaric acid in IPA (2.3 ml, 0.9 equiv). This solution was stirred at room temperature for 15 minutes then concentrated under reduced pressure. The residue was suspended in acetonitrile (10 ml), warmed to reflux then cooled to room temperature. The resulting solid was filtered and washed with cold acetonitrile to provide (2S)-2-[(1S)-(4-chloro-2-methoxyphenoxy)(pyridin-2-yl)methyl]morpholine as the fumaric acid salt (0.160 g). ¹H NMR (400 MHz, METHANOL-D4) δ ppm 3.1 (td, J=12.4, 3.8 Hz, 1H) 3.2 (m, 1H) 3.2 (m, 2H) 3.7 (m, 4H) 4.0 (ddd, J=12.7, 4.0, 1.1 Hz, 1H) 4.2 (dt, J=8.8, 4.5 Hz, 1H) 5.3 (d, J=4.4 Hz, 1H) 6.6 (dd, J=9.1, 2.9 Hz, 1H) 6.6 (s, 2H) 6.7 (m, 1H) 6.9 (d, J=2.8 Hz, 1H) 7.3 (ddd, J=7.6, 4.9, 1.1 Hz, 1H) 7.5 (dt, J=7.9, 0.9 Hz, 1H) 7.8 (td, J=7.7, 1.9 Hz, 1H) 8.5 (ddd, J=4.9, 1.7, 0.9 Hz, 1H). MS (APCI) 335.1 (M+1).

### EXAMPLES 104-106

The compounds of Examples 104-106 were made in a manner analogous to the synthesis of the compound of Example 103 ((2S)-2-[(1S)-(4-chloro-2-methoxyphenoxy)(pyridin-2-yl)methyl]morpholine as the fumaric acid salt).

| Ex. | |
|---|---|
| No. | Compound |
| 104 | (2S)-2-[(1S)-(4-chloro-2-fluorophenoxy)(pyridin-2- |
| | yl)methyl]morpholine fumarate |
| 105 | (2S)-2-[(1S)-(2-chloro-4-fluorophenoxy)(pyridin-2- |
| | yl)methyl]morpholine fumarate |
| 106 | (2S)-2-[(1S)-(2-chloro-4-methoxyphenoxy)(pyridin-2- |
| | yl)methyl]morpholine fumarate |

### EXAMPLE 107

### (2S)-2-[(1R)-(4-chloro-2-methoxyphenoxy) (phenyl)methyl]morpholine hydrochloride

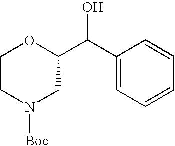

Tert-butyl (2S)-2-benzoylmorpholine-4-carboxylate (1.4 g, 4.8 mmol) was dissolved in EtOH (50 ml) and cooled in an ice bath. Then NaBH₄ (0.41 g, 10.8 mmol) was added in one portion and stirred the mixture at 0° C. for 30 minutes then quenched with saturated aqueous NH₄Cl (50 ml). The mixture was stirred for 5 minutes then warmed to room temperature. The mixture was then extracted three times with 100 ml of diethylether. The combined organic layers were dried over Na₂SO₄, filtered and concentrated under reduced pressure to provide tert-butyl (2S)-[(2R)-[hydroxy(phenyl)methyl]]morpholine-4-carboxylate and tert-butyl (2S)-[(2S)-[hydroxy(phenyl)methyl]]morpholine-4-carboxylate in a 2.5 to 1 ratio.

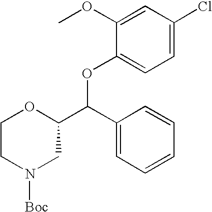

Tert-butyl (2S)-2-[hydroxy(phenyl)methyl]morpholine-4-carboxylate from above (1.4 g, 4.8 mmol) was combined with triphenylphosphine (3.3 g, 12 mmol) and 4-chloro-2-methoxyphenol (3.0 g, 19 mmol) in 45 ml toluene and cooled in an ice bath. Diisopropylazodicarboxylate (2.3 ml, 12 mmol) was added dropwise and then the mixture was warmed slowly to room temperature and stirred for 18 hours. The mixture was concentrated under reduced pressure and the residue purified by silica gel chromatography eluting with 5%-30% EtOAc in hexanes, providing tert-butyl (2S)-2-[(R)-(4-chloro-2-methoxyphenoxy)(phenyl)methyl]morpholine-4-carboxylate and tert-butyl (2S)-2-[(S)-(4-chloro-2-methoxyphenoxy)(phenyl)methyl]morpholine-4-carboxylate separately as clear oils.

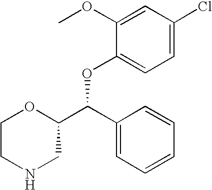

Tert-butyl (2S)-2-[(R)-(4-chloro-2-methoxyphenoxy)(phenyl)methyl]morpholine-4-carboxylate from above (1.0 g, 2.3 mmol) was dissolved in CH₂Cl₂ (10 ml) and treated with 2M HCl in ether (3 ml, 6 mmol), and then stirred at room temperature for 18 hours. Concentration under reduced pressure and recrystallization from EtOAc/MeOH provided (2S)-2-[(1R)-(4-chloro-2-methoxyphenoxy)(phenyl)methyl]morpholine hydrochloride as a white solid. ¹H NMR (400 MHz, METHANOL-D4) δ ppm 3.2 (m, 3H) 3.6 (m, 1H) 3.7 (td, J=12.6, 3.4 Hz, 1H) 3.9 (s, 3H) 4.1 (m, 2H) 5.2 (d, J=6.2 Hz, 1H) 6.7 (m, 2H) 7.0 (d, J=2.0 Hz, 1H) 7.3 (m, 5H). MS (APCI) 334.1 (M+1).

### EXAMPLE 108

### (2R)-2-[(1S)-(4-chloro-2-methoxyphenoxy) (phenyl)methyl]morpholine hydrochloride

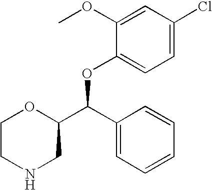

(2R)-2-[(1S)-(4-chloro-2-methoxyphenoxy)(phenyl)methyl]morpholine hydrochloride was prepared in a manner similar to the preparation of the compound of Example 107 using tert-butyl (2R)-2-benzoylmorpholine-4-carboxylate. ¹H NMR (400 MHz, METHANOL-D4) δ ppm 3.2 (m, 3H) 3.6 (m, 1H) 3.7 (td, J=12.6, 3.4 Hz, 1H) 3.9 (s, 3H) 4.1 (m, 2H) 5.2 (d, J=6.2 Hz, 1H) 6.7 (m, 2H) 7.0 (d, J=2.0 Hz, 1H) 7.3 (m, 5H). MS (APCI) 334.1 (M+1).

### EXAMPLE 109

### (2R)-2-[(1R)-(4-chloro-2-methoxyphenoxy) (phenyl)methyl]morpholine succinate

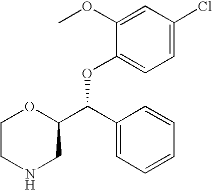

Tert-butyl (2R)-2-[(1R)-(4-Chloro-2-methoxy-phenoxy)-phenyl-methyl]-morpholine-4-carboxylate was prepared in a manner similar to the preparation of tert-butyl (2S)-2-[(R)-(4-chloro-2-methoxyphenoxy)(phenyl)methyl]morpholine-4-carboxylate in Example 107 using tert-butyl (2R)-2-benzoylmorpholine-4-carboxylate. Tert-butyl (2R)-2-[(1R)-(4-Chloro-2-methoxy-phenoxy)-phenyl-methyl]-morpholine-4-carboxylate was dissolved in CH₂Cl₂. 2M HCl in Et₂O was added to the solution and stirred overnight at room temperature. The reaction was diluted with CH₂Cl₂ and neutralized with 5% NaOH. Silica gel chromatography (5% MeOH:CH₂Cl₂. 1000 mL) of the material afforded (2R)-2-[(1R)-(4-chloro-2-methoxyphenoxy) (phenyl)methyl]morpholine as a clear oil (230 mg). The oil was dissolved in about 5 ml of diethylether. A 1 ml solution of succinic acid (81 mg) was added, and the mixture was stirred at room temperature. A precipitate formed after about 5 minutes. The precipitate was filtered and washed with diethylether and dried in a vacuum oven to provide 256 mg of(2R)-2-[(1R)-(4-chloro-2-methoxyphenoxy) (phenyl)methyl]morpholine succinate as a white solid. ¹H NMR (400 MHz, METHANOL-D4) δ ppm 3.1 (m, 3H) 3.2 (m, 1H) 3.8 (ddd, J=13.0, 12.0, 2.5 Hz, 1H) 3.9 (s, 3H) 4.1 (ddd, J=10.8, 5.0, 2.5 Hz, 2H) 5.3 (d, J=5.1 Hz, 1H) 6.7 (m, 1H) 6.7 (m, 1H) 7.0 (d, J=2.3 Hz, 1H) 7.4 (m, 5H). MS (APCI) 334.1 (M+1).

### EXAMPLES 37-109

| | MS and | |
|---|---|---|
| | Combustion | |
| | analysis (CHN) | |
| Ex. | (Calculated, | |
| No. | Experimental) | NMR |
| 37 | [M + 1] = 340 | ¹H NMR (400 MHz, CHLOROFORM-D) □ ppm 3.00 (s, 2H) 3.29 (d, |
| | | J = 12.28 Hz, 1H) 3.40 (d, J = 11.89 Hz, 1H) 4.06 (m, 2H) 4.41 (m, 1H) |
| | | 5.17 (d, J = 3.70 Hz, 1H) 6.63 (m, 1H) 6.75 (m, 1H) 7.03 (m, 1H) 7.09 (m, 3H) |
| | | 7.33 (m, 1H) 10.17 (s, 2H). |
| 38 | MS (APCI) | ¹H NMR (400 MHz, METHANOL-D4) d ppm 3.1 (m, 3H) 3.2 (m, 1H) 3.8 (m, |
| | M + 1 = 334.1 | 1H) 3.8 (s, 3H) 4.1 (m, J = 10.9, 5.3, 3.1, 2.8 Hz, 2H) 5.3 (d, J = 4.9 Hz, 1H) |
| | | 6.7 (m, 2H) 6.9 (d, J = 2.2 Hz, 1H) 7.3 (m, 5H) |
| 39 | [M + 1] = 324 | ¹H NMR (400 MHz, CHLOROFORM-D) □ ppm 3.00 (m, 2H) 3.34 (m, 2H) |
| | | 4.08 (m, 2H) 4.41 (d, J = 7.80 Hz, 1H) 5.22 (d, J = 2.53 Hz, 1H) 6.54 (t, |
| | | J = 7.60 Hz, 1H) 6.80 (m, 2H) 7.03 (m, 1H) 7.11 (m, 2H) 7.33 (m, 1H) |
| | | 10.17 (bs, 2H) |
| 40 | MS (APCI) | ¹H NMR (400 MHz, DMSO-D6) d ppm 2.2 (s, 3H) 2.9 (m, 3H) 3.2 (d, |
| | M + 1 = 314.2 | J = 12.5 Hz, 1H) 3.7 (ddd, J = 12.3, 2.3 Hz, 1H) 3.8 (s, 3H) 4.0 (dd, J = 13.2, |
| | | 3.0 Hz, 1H) 4.1 (m, 1H) 5.3 (d, J = 5.1 Hz, 1H) 6.5 (ddd, J = 8.2, 2.0, 0.8 Hz, |
| | | 1H) 6.7 (d, J = 8.2 Hz, 1H) 6.8 (d, J = 1.8 Hz, 1H) 7.3 (m, 5H) 9.1 (bs, 2H) |
| 41 | [M + 1] = 340 | ¹H NMR (400 MHz, CHLOROFORM-D) □ ppm 3.00 (d, J = 1.76 Hz, 2H) |
| | | 3.30 (d, J = 11.91 Hz, 1H) 3.40 (d, J = 11.91 Hz, 1H) 4.06 (m, 2H) 4.41 (dd, |
| | | J = 9.96, 2.93 Hz, 1H) 5.21 (d, J = 3.90 Hz, 1H) 6.41 (dd, J = 9.96, 2.73 Hz, 1H) |
| | | 6.61 (ddd, J = 8.78, 7.81, 2.73 Hz, 1H) 7.06 (m, 3H) 7.31 (m, 2H) |
| | | 10.16 (s, 2H) |
| 42 | M + 1 (332) | ¹H NMR (400 MHz, CHLOROFORM-D) d ppm 2.2 (s, 3H) 3.1 (m, 1H) |
| | C (62.04, 61.85), | 3.2 (t, J = 10.1 Hz, 1H) 3.3 (d, J = 10.2 Hz, 1H) 3.4 (d, J = 12.1 Hz, 1H) 3.8 (s, 3H) |
| | H (6.30, 6.21), | 4.0 (t, J = 12.0 Hz, 1H) 4.1 (m, 1H) 4.3 (d, J = 10.3 Hz, 1H) 5.1 (d, J = 3.7 Hz, |
| | N (3.81, 3.66) | 1H) 6.5 (m, 2H) 6.7 (s, 1H) 7.0 (t, J = 8.6 Hz, 2H) 7.3 (m, 2H) |
| 43 | M + 1 (332) | ¹H NMR (400 MHz, CHLOROFORM-D) d ppm 2.2 (s, 3H) 3.1 (d, J = 12.1 Hz, |
| | C (62.04, 61.93) | 1H) 3.2 (m, 1H) 3.3 (d, J = 12.3 Hz, 1H) 3.4 (d, J = 12.1 Hz, 1H) 3.8 (s, 3H) |
| | H (6.30, 6.6.22), | 4.0 (t, J = 12.1 Hz, 1H) 4.1 (m, 1H) 4.3 (d, J = 10.5 Hz, 1H) 5.1 (d, J = 3.9 Hz, |
| | N (3.81, 3.74), | 1H) 6.5 (m, 2H) 6.7 (s, 1H) 7.0 (t, J = 8.6 Hz, 2H) 7.3 (m, 2H) |
| | Cl (9.64, 9.66) | |
| 44 | [M + 1] = 340. | ¹H NMR (400 MHz, CHLOROFORM-D) □ ppm 3.00 (m, 2H) 3.32 (m, 2H) |
| | | 4.06 (m, 2H) 4.38 (d, J = 7.42 Hz, 1H) 5.15 (d, J = 3.51 Hz, 1H) 6.69 (t, |
| | | J = 8.78 Hz, 1H) 6.88 (dt, J = 8.88, 1.90 Hz, 1H) 7.06 (m, 4H) 7.32 (m, 1H) |
| | | 10.13 (s, 2H) |
| 45 | [M + 1] = 352 | ¹H NMR (400 MHz, CHLOROFORM-D) □ ppm 3.08 (m, 2H) 3.29 (m, 1H) |
| | | 3.37 (m, 1H) 3.84 (s, 3H) 4.05 (m, 2H) 4.32 (m, 1H) 5.09 (s, 1H) 6.59 (d, |
| | | J = 8.59 Hz, 1H) 6.68 (m, 1H) 6.82 (d, J = 2.34 Hz, 1H) 7.00 (m, 1H) 7.09 (d, |
| | | J = 8.39 Hz, 2H) 7.30 (td, J = 7.81, 5.86 Hz, 1H) 10.11 (s, 2H) |
| 46 | [M + 1] = 336 | ¹H NMR (400 MHz, CHLOROFORM-D) □ ppm 3.06 (m, 1H) 3.15 (m, 1H) |
| | | 3.29 (m, 1H) 3.38 (m, 1H) 3.84 (s, 3H) 4.06 (m, 2H) 4.32 (m, 1H) 5.05 (s, |
| | | 1H) 6.40 (td, J = 8.35, 2.83 Hz, 1H) 6.61 (m, 2H) 7.00 (td, J = 8.35, 2.05 Hz, |
| | | 1H) 7.11 (t, J = 7.13 Hz, 2H) 7.29 (m, 1H) 10.12 (m, 2H) |
| 47 | [M + 1] = 324. | ¹H NMR (400 MHz, METHANOL-D4) d ppm 3.14 (m, 3H) 3.26 (d, J = 12.88 Hz, |
| | | 1H) 3.82 (td, J = 12.59, 2.73 Hz, 1H) 4.16 (m, 2H) 5.36 (d, J = 5.27 Hz, 1H) |
| | | 6.90 (m, 2H) 7.00 (m, 1H) 7.08 (m, 1H) 7.24 (m, 2H) 7.36 (m, 1H) |
| | | (NH-proton obscured by solvent peak.) |
| 48 | [M + 1] = 358 | ¹H NMR (400 MHz, CHLOROFORM-D) □ ppm 2.98 (m, 2H) 3.33 (m, 2H) |
| | | 4.07 (m, 2H) 4.42 (m, 1H) 5.21 (d, J = 4.10 Hz, 1H) 6.25 (dt, J = 9.86, 2.10 Hz, |
| | | 1H) 6.54 (td, J = 8.64, 2.64 Hz, 1H) 7.08 (m, 3H) 7.36 (ddd, J = 8.98, |
| | | 7.81, 5.66 Hz, 1H) 10.20 (m, 2H) |
| 49 | [M + 1] = 302 | ¹H NMR (400 MHz, CHLOROFORM-D) □ ppm 2.31 (s, 3H) 2.95 (m, 2H) |
| | | 3.27 (d, J = 12.10 Hz, 1H) 3.34 (m, 1H) 4.04 (d, J = 9.76 Hz, 2H) 4.36 (m, 1H) |
| | | 5.21 (d, J = 3.90 Hz, 1H) 6.51 (d, J = 8.20 Hz, 1H) 6.83 (m, 1H) 6.98 (m, 2H) |
| | | 7.05 (d, J = 9.37 Hz, 1H) 7.11 (t, J = 6.05 Hz, 2H) 7.30 (m, 1H) 10.13 (m, |
| | | 2H) |
| 50 | [M + 1] = 336 | ¹H NMR (400 MHz, CHLOROFORM-D) □ ppm 3.05 (m, 2H) 3.28 (d, |
| | | J = 12.88 Hz, 1H) 3.39 (m, 1H) 3.80 (s, 3H) 4.08 (m, 2H) 4.35 (dd, |
| | | J = 10.35, 3.12 Hz, 1H) 5.27 (d, J = 4.49 Hz, 1H) 6.61 (m, 2H) 6.90 (td, |
| | | J = 8.39, 6.05 Hz, 1H) 6.98 (td, J = 8.30, 2.15 Hz, 1H) 7.15 (d, J = 7.61 Hz, 1H) |
| | | 7.25 (m, 2H) 10.11 (m, 2H) |
| 51 | [M + 1] = 332 | ¹H NMR (400 MHz, CHLOROFORM-D) □ ppm 2.13 (s, 3H) 3.08 (m, 1H) |
| | | 3.15 (m, 1H) 3.29 (d, J = 12.49 Hz, 1H) 3.42 (d, J = 11.91 Hz, 1H) 3.82 (s, 3H) |
| | | 4.03 (m, 2H) 4.31 (d, J = 8.78 Hz, 1H) 5.14 (d, J = 3.32 Hz, 1H) 6.50 (s, 1H) |
| | | 6.72 (m, 2H) 6.99 (m, 1H) 7.14 (m, 2H) 7.30 (m, 1H) 10.02 (bs, 1H) |
| | | 10.19 (bs, 1H) |
| 52 | [M + 1] = 352.1 | ¹H NMR (400 MHz, METHANOL-D4) □ ppm 3.15 (m, 2H) 3.24 (m, 1H) |
| | | 3.30 (m, 1H) 3.78 (m, 1H) 3.85 (s, 3H) 4.12 (m, 2H) 4.83 (b, 2H) 5.27 (d, |
| | | J = 4.29 Hz, 1H) 6.45 (td, J = 8.43, 2.83 Hz, 1H) 6.77 (m, 2H) 7.32 (m, 3H) |
| | | 7.46 (s, 1H) |
| 53 | [M + 1] = 356.0 | ¹H NMR (400 MHz, METHANOL-D4) □ ppm 3.11 (m, 2H) 3.25 (m, 1H) |
| | | 3.48 (m, 1H) 3.82 (td, J = 12.62, 2.44 Hz, 1H) 4.17 (m, 2H) 4.84 (b, 2H) |
| | | 5.55 (d, J = 4.87 Hz, 1H) 6.70 (m, 2H) 7.37 (m, 3H) 7.45 (m, 2H) |
| 54 | [M + 1] = 348.1 | ¹H NMR (400 MHz, METHANOL-D4) □ ppm 2.31 (s, 3H) 3.10 (m, 2H) |
| | | 3.24 (m, 1H) 3.48 (m, 1H) 3.81 (m, 1H) 3.85 (s, 3H) 4.13 (ddd, J = 10.62, 5.07, |
| | | 2.63 Hz, 2H) 4.84 (b, 2H) 5.25 (d, J = 5.26 Hz, 1H) 6.70 (m, 2H) 6.95 (d, |
| | | J = 2.14 Hz, 1H) 7.18 (m, 4H) |
| 55 | [M + 1] = 328.2 | ¹H NMR (400 MHz, METHANOL-D4) □ ppm 2.20 (s, 3H) 2.31 (s, 3H) |
| | | 3.10 (m, 2H) 3.21 (m, 2H) 3.78 (dd, J = 12.96, 2.44 Hz, 1H) 3.83 (s, 3H) |
| | | 4.11 (m, 2H) 4.84 (b, 2H) 5.20 (d, J = 5.07 Hz, 1H) 6.51 (m, 1H) 6.62 (d, J = 8.38 Hz, |
| | | 1H) 6.77 (s, 1H) 7.11 (m, 1H) 7.19 (m, 3H) |
| 56 | [M + 1] = 336.1 | ¹H NMR (400 MHz, METHANOL-D4) □ ppm 2.32 (s, 3H) 3.05 (m, 1H) |
| | | 3.12 (m, 2H) 3.26 (m, 1H) 3.83 (td, J = 12.62, 2.63 Hz, 1H) 4.17 (m, 2H) 4.84 (b, |
| | | 2H) 5.37 (d, J = 5.46 Hz, 1H) 6.86 (m, 2H) 7.17 (td, J = 8.14, 5.36 Hz, 3H) |
| | | 7.25 (m, 2H) |
| 57 | [M + 1] = 332.1 | ¹H NMR (400 MHz, METHANOL-D4) □ppm 2.31 (s, 3H) 3.12 (m, 3H) |
| | | 3.24 (m, 1H) 3.81 (m, 1H) 3.84 (s, 3H) 4.12 (ddd, J = 10.38, 5.02, 2.73 Hz, 2H) |
| | | 4.84 (b, 2H) 5.19 (d, J = 5.07 Hz, 1H) 6.42 (td, J = 8.48, 2.92 Hz, 1H) |
| | | 6.74 (m, 2H) 7.13 (d, J = 7.21 Hz, 1H) 7.21 (m, 3H) |
| 58 | [M + 1] = 344.1 | ¹H NMR (400 MHz, METHANOL-D4) □ ppm 2.31 (s, 3H) 3.13 (m, 3H) |
| | | 3.21 (m, 1H) 3.67 (s, 3H) 3.80 (m, 1H) 3.82 (s, 3H) 4.12 (m, 2H) 4.83 (b, 2H) |
| | | 5.12 (d, J = 5.07 Hz, 1H) 6.24 (dd, J = 8.77, 2.92 Hz, 1H) 6.52 (d, J = 2.92 Hz, |
| | | 1H) 6.67 (d, J = 8.77 Hz, 1H) 7.11 (d, J = 7.21 Hz, 1H) 7.20 (m, 3H) |
| 59 | [M + 1] = 336.1 | ¹H NMR (400 MHz, METHANOL-D4) □ppm 2.33 (s, 3H) 3.09 (m, 3H) |
| | | 3.23 (m, 1H) 3.83 (td, J = 12.62, 2.24 Hz, 1H) 4.17 (m, 2H) 4.84 (b, 2H) 5.43 (d, |
| | | J = 5.46 Hz, 1H) 6.66 (m, 2H) 7.21 (m, 2H) 7.27 (m, 2H) 7.34 (m, 1H) |
| 60 | [M + 1] = 340, | ¹H NMR (400 MHz, CHLOROFORM-D) d ppm 2.88 (m, 1H) 2.98 (m, 1H) |
| | 342 | 3.25 (d, J = 12.30 Hz, 2H) 4.06 (d, J = 7.42 Hz, 2H) 4.42 (dd, J = 10.64, 4.98 Hz, |
| | | 1H) 5.34 (d, J = 5.27 Hz, 1H) 6.89 (m, 2H) 7.00 (td, J = 8.35, 2.44 Hz, 1H) |
| | | 7.07 (m, 1H) 7.16 (m, 2H) 7.27 (td, J = 8.05, 5.76 Hz, 1H) 10.14 (bs, 2H). |
| 61 | [M + 1] = 364.1 | ¹H NMR (400 MHz, METHANOL-D4) □ ppm 3.11 (m, 3H) 3.22 (m, 1H) |
| | | 3.76 (s, 3H) 3.81 (m, 1H) 3.85 (s, 3H) 4.13 (m, 2H) 4.83 (b, 2H) 5.27 (d, |
| | | J = 5.07 Hz, 1H) 6.72 (m, 2H) 6.88 (dd, J = 8.48, 2.44 Hz, 1H) 6.96 (m, 3H) |
| | | 7.26 (t, J = 7.80 Hz, 1H) |
| 62 | [M + 1] = 344.2 | ¹H NMR (400 MHz, METHANOL-D4) □ ppm 2.21 (s, 3H) 3.12 (m, 3H) |
| | | 3.22 (m, 1H) 3.76 (s, 3H) 3.81 (m, 1H) 3.84 (s, 3H) 4.13 (m, 2H) 4.83 (b, 2H) |
| | | 5.23 (d, J = 4.87 Hz, 1H) 6.52 (d, J = 8.19 Hz, 1H) 6.65 (d, J = 8.19 Hz, 1H) |
| | | 6.77 (d, J = 1.56 Hz, 1H) 6.85 (dd, J = 8.19, 2.34 Hz, 1H) 6.97 (m, 2H) |
| | | 7.24 (t, J = 7.90 Hz, 1H) |
| 63 | [M + 1] = 352.1 | ¹H NMR (400 MHz, METHANOL-D4) □ ppm 3.05 (m, 1H) 3.13 (m, 2H) |
| | | 3.26 (m, 1H) 3.77 (s, 3H) 3.84 (m, 1H) 4.17 (m, 2H) 4.84 (b, 2H) 5.40 (d, |
| | | J = 5.26 Hz, 1H) 6.88 (m, 3H) 6.97 (dd, J = 3.90, 2.14 Hz, 2H) 7.18 (dt, |
| | | J = 8.14, 1.39 Hz, 1H) 7.27 (m, 1H) |
| 64 | [M + 1] = 324 | ¹H NMR (400 MHz, CHLOROFORM-D) d ppm 3.01 (m, 2H) 3.32 (m, 2H) |
| | | 4.07 (m, 2H) 4.38 (m, 1H) 5.10 (d, J = 3.90 Hz, 1H) 6.63 (m, 1H) 6.73 (m, 1H) |
| | | 6.81 (ddd, J = 11.06, 8.24, 2.92 Hz, 1H) 7.03 (td, J = 8.29, 2.34 Hz, 1H) |
| | | 7.10 (m, 2H) 7.32 (td, J = 7.99, 5.85 Hz, 1H) 10.16 (s, 2H) |
| 65 | [M + 1] = 342 | ¹H NMR (400 MHz, CHLOROFORM-D) d ppm 3.00 (m, 2H) 3.25 (m, 2H) |
| | | 4.10 (m, 2H) 4.38 (m, 1H) 5.13 (d, J = 4.68 Hz, 1H) 6.59 (m, 2H) 7.04 (m, 1H) |
| | | 7.14 (m, 2H) 7.30 (m, 1H) 10.18 (s, 2H). |
| 66 | [M + 1] = 330 | ¹H NMR (400 MHz, CHLOROFORM-D) d ppm 0.99 (t, J = 5.56 Hz, 3H) |
| | | 1.67 (m, 2H) 2.67 (m, 2H) 2.95 (m, 2H) 3.26 (m, 2H) 4.07 (m, 2H) 4.38 (dd, |
| | | J = 2.44, 1.46 Hz, 1H) 5.19 (s, 1H) 6.52 (d, J = 7.81 Hz, 1H) 6.85 (t, J = 7.32 Hz, |
| | | 1H) 6.98 (m, 2H) 7.05 (d, J = 8.39 Hz, 1H) 7.12 (d, J = 7.22 Hz, 2H) |
| | | 7.30 (m, 1H) 10.12 (s, 2H) |
| 67 | [M + 1] = 356 | ¹H NMR (400 MHz, CHLOROFORM-D) d ppm 2.98 (m, 2H) 3.32 (m, 2H) |
| | | 4.07 (m, 2H) 4.37 (m, 1H) 5.24 (s, 1H) 6.87 (d, J = 8.39 Hz, 2H) 7.06 (m, 3H) |
| | | 7.33 (m, 1H) 7.46 (d, J = 8.00 Hz, 2H) 10.18 (s, 2H) |
| 68 | [M + 1] = 348.1 | ¹H NMR (400 MHz, METHANOL-D4) □ ppm 3.11 (m, 2H) 3.23 (m, 1H) |
| | | 3.76 (s, 3H) 3.80 (dd, J = 12.96, 2.63 Hz, 2H) 3.85 (s, 3H) 4.13 (m, 2H) |
| | | 4.83 (b, 2H) 5.21 (d, J = 5.07 Hz, 1H) 6.43 (m, 1H) 6.76 (m, 2H) 6.87 (m, 1H) |
| | | 6.97 (m, 2H) 7.25 (t, J = 7.90 Hz, 1H) |
| 69 | [M + 1] = 352.1 | ¹H NMR (400 MHz, METHANOL-D4) □ ppm 3.10 (m, 3H) 3.26 (m, 1H) |
| | | 3.77 (s, 3H) 3.84 (td, J = 12.67, 2.53 Hz, 1H) 4.17 (m, 2H) 4.83 (b, 2H) |
| | | 5.46 (d, J = 5.46 Hz, 1H) 6.65 (m, 1H) 6.73 (dd, J = 10.43, 2.83 Hz, 1H) |
| | | 6.91 (ddd, J = 8.29, 2.53, 0.88 Hz, 1H) 6.98 (m, 2H) 7.32 (m, 2H) |
| 70 | [M + 1] = 396.0 | ¹H NMR (400 MHz, METHANOL-D4) □ ppm 3.09 (m, 2H) 3.20 (s, 1H) |
| | | 3.26 (d, J = 12.87 Hz, 1H) 3.76 (s, 3H) 3.84 (td, J = 12.67, 2.53 Hz, 1H) 4.18 (m, 2H) |
| | | 4.83 (b, 2H) 5.42 (d, J = 5.26 Hz, 1H) 6.90 (m, 3H) 6.97 (dd, J = 4.48, |
| | | 2.92 Hz, 2H) 7.31 (m, 2H) |
| 71 | [M + 1] = 352.1 | ¹H NMR (400 MHz, METHANOL-D4) □ ppm 3.13 (m, 2H) 3.25 (m, 2H) |
| | | 3.78 (m, 1H) 3.83 (s, 3H) 4.11 (m, 2H) 4.83 (b, 2H) 5.25 (d, J = 3.70 Hz, 1H) |
| | | 6.43 (td, J = 8.53, 2.83 Hz, 1H) 6.75 (m, 2H) 7.37 (m, 4H) |
| 72 | [M + 1] = 356.0 | 1H NMR (400 MHz, METHANOL-D4) □ ppm 3.10 (m, 2H) 3.24 (m, 2H) |
| | | 3.83 (m, 1H) 4.10 (d, J = 0.78 Hz, 1H) 4.21 (m, 1H) 4.83 (b, 2H) 5.47 (s, 1H) |
| | | 6.88 (m, 2H) 7.17 (d, J = 7.80 Hz, 1H) 7.37 (t, J = 7.51 Hz, 4H) |
| 73 | [M + 1] = 368.0 | ¹H NMR (400 MHz, METHANOL-D4) □ ppm 3.11 (m, 2H) 3.22 (m, 2H) |
| | | 3.78 (m, 1H) 3.84 (s, 3H) 4.10 (m, 2H) 4.83 (b, 2H) 5.32 (s, 1H) 6.70 (m, |
| | | 2H) 6.95 (d, J = 1.17 Hz, 1H) 7.36 (m, 4H) |
| 74 | [M + 1] = 340 | ¹H NMR (400 MHz, CHLOROFORM-D) d ppm 3.00 (m, 2H) 3.34 (m, 2H) |
| | | 4.06 (m, 2H) 4.39 (d, J = 5.85 Hz, 1H) 5.16 (s, 1H) 6.62 (dd, J = 9.06, 4.78 Hz, |
| | | 1H) 6.74 (m, 1H) 7.06 (m, 3H) 7.31 (dd, J = 8.09, 5.17 Hz, 2H) |
| | | 10.14 (s, 2H) |
| 75 | [M + 1] = 348.1 | ¹H NMR (400 MHz, METHANOL-D4) □ ppm 2.21 (s, 3H) 3.12 (m, 2H) |
| | | 3.23 (m, 1H) 3.78 (m, 2H) 3.83 (s, 3H) 4.11 (m, 2H) 4.83 (b, 2H) 5.27 (d, |
| | | J = 4.48 Hz, 1H) 6.52 (dt, J = 8.14, 1.00 Hz, 1H) 6.63 (d, J = 7.99 Hz, 1H) |
| | | 6.78 (d, J = 1.75 Hz, 1H) 7.36 (m, 4H) |
| 76 | [M + 1] = 356.0 | ¹H NMR (400 MHz, METHANOL-D4) □ ppm 3.13 (m, 4H) 3.81 (m, 1H) |
| | | 4.14 (m, 2H) 4.83 (b, 2H) 5.41 (d, J = 4.87 Hz, 1H) 6.92 (m, 2H) 7.17 (dd, |
| | | J = 11.01, 2.24 Hz, 1H) 7.39 (m, 4H) |
| 77 | [M + 1] = 417.9 | ¹H NMR (400 MHz, METHANOL-D4) □ ppm 3.09 (m, 2H) 3.26 (m, 2H) |
| | | 3.82 (td, J = 12.62, 2.44 Hz, 1H) 4.12 (dd, J = 13.06, 3.51 Hz, 1H) 4.18 (ddd, |
| | | J = 11.26, 4.73, 2.14 Hz, 1H) 4.84 (s, 2H) 5.53 (d, J = 4.87 Hz, 1H) 6.83 (d, |
| | | J = 8.97 Hz, 1H) 7.15 (dd, J = 8.97, 2.53 Hz, 1H) 7.39 (s, 4H) 7.56 (d, J = 2.53 Hz, |
| | | 1H) |
| 78 | [M + 1] = 313. | ¹H NMR (400 MHz, CHLOROFORM-D) d ppm 3.00 (m, 2H) 3.35 (d, |
| | | J = 11.31 Hz, 1H) 3.54 (d, J = 7.80 Hz, 1H) 4.07 (m, 2H) 4.49 (d, J = 7.60 Hz, |
| | | 1H) 5.40 (s, 1H) 6.73 (d, J = 8.58 Hz, 1H) 7.02 (m, 2H) 7.11 (d, J = 8.38 Hz, |
| | | 1H) 7.17 (d, J = 7.41 Hz, 1H) 7.35 (m, 2H) 7.53 (dd, J = 7.70, 1.27 Hz, 1H) |
| | | 10.05 (bs, 1H) 10.25 (bs, 1H) |
| 79 | [M + 1] = 332 | ¹H NMR (400 MHz, CHLOROFORM-D) □ ppm 2.23 (s, 3H) 3.07 (m, 1H) |
| | | 3.19 (m, 1H) 3.29 (d, J = 12.30 Hz, 1H) 3.42 (d, J = 12.30 Hz, 1H) 3.84 (m, 3H) |
| | | 4.00 (t, J = 11.71 Hz, 1H) 4.08 (m, 1H) 4.30 (d, J = 9.37 Hz, 1H) 5.08 (d, |
| | | J = 2.93 Hz, 1H) 6.50 (dd, J = 8.20, 1.17 Hz, 1H) 6.56 (m, 1H) 6.66 (d, |
| | | J = 1.56 Hz, 1H) 6.98 (m, 1H) 7.13 (m, 2H) 7.29 (m, 1H) 10.03 (m, 1H) |
| | | 10.20 (m, 1H) |
| 80 | [M + 1] = 322.1 | ¹H NMR (400 MHz, METHANOL-D4) □ ppm 3.03 (m, 2H) 3.11 (dd, |
| | | J = 12.20, 4.00 Hz, 1H) 3.22 (dt, J = 12.93, 1.24 Hz, 1H) 3.82 (td, J = 12.54, |
| | | 2.64 Hz, 1H) 4.11 (dd, J = 13.08, 3.12 Hz, 1H) 4.17 (ddd, J = 9.66, 5.66, 3.81 Hz, |
| | | 1H) 4.86 (b, 3H) 5.39 (d, J = 5.47 Hz, 1H) 6.67 (s, 2H) 6.88 (m, 2H) |
| | | 6.96 (m, 1H) 7.37 (m, 5H) |
| 81 | [M + 1] = 338.0 | ¹H NMR (400 MHz, METHANOL-D4) □ ppm 3.04 (m, 2H) 3.12 (m, 1H) |
| | | 3.21 (m, 1H) 3.82 (td, J = 12.54, 2.44 Hz, 1H) 4.10 (dd, J = 12.88, 3.51 Hz, 1H) |
| | | 4.18 (ddd, J = 11.13, 5.27, 2.34 Hz, 1H) 4.85 (b, 3H) 5.49 (d, J = 5.27 Hz, |
| | | 1H) 6.68 (s, 2H) 6.84 (dd, J = 6.15, 3.61 Hz, 1H) 7.04 (m, 2H) 7.35 (m, 5H) |
| 82 | [M + 1] = 318.1 | ¹H NMR (400 MHz, METHANOL-D4) □ ppm 2.36 (s, 3H) 2.96 (dd, J = 12.59, |
| | | 11.22 Hz, 1H) 3.08 (m, 2H) 3.21 (m, 1H) 3.82 (td, J = 12.49, 2.54 Hz, 1H) |
| | | 4.11 (m, 1H) 4.16 (ddd, J = 8.35, 5.51, 2.73 Hz, 1H) 4.85 (b, 3H) 5.35 (d, |
| | | J = 5.47 Hz, 1H) 6.65 (m, 1H) 6.68 (s, 2H) 6.90 (m, 2H) 7.34 (m, 5H) |
| 83 | [M + 1] = 340.1 | ¹H NMR (400 MHz, METHANOL-D4) □ ppm 3.06 (m, 3H) 3.22 (m, 1H) |
| | | 3.82 (td, J = 12.59, 2.54 Hz, 1H) 4.11 (dd, J = 12.88, 3.32 Hz, 1H) 4.19 (ddd, |
| | | J = 10.59, 5.61, 2.93 Hz, 1H) 4.86 (b, 3H) 5.50 (d, J = 5.66 Hz, 1H) 6.61 (m, |
| | | 4H) 7.37 (m, 5H) |
| 84 | [M + 1] = 334.1 | ¹H NMR (400 MHz, METHANOL-D4) □ ppm 3.08 (m, 3H) 3.21 (m, 1H) |
| | | 3.79 (dd, J = 12.79, 2.64 Hz, 1H) 3.84 (s, 3H) 4.13 (m, 2H) 4.85 (b, 3H) |
| | | 5.32 (d, J = 5.08 Hz, 1H) 6.67 (s, 2H) 6.77 (d, J = 2.34 Hz, 1H) 6.87 (m, 2H) |
| | | 7.36 (m, 5H) |
| 85 | [M + 1] = 360.1 | ¹H NMR (400 MHz, METHANOL-D4) □ ppm 2.79 (m, 1H) 2.94 (m, 1H) |
| | | 3.08 (td, J = 12.49, 3.90 Hz, 1H) 3.19 (m, 1H) 3.82 (td, J = 12.40, 2.54 Hz, 1H) |
| | | 4.11 (d, J = 15.81 Hz, 1H) 4.22 (ddd, J = 10.98, 6.78, 2.34 Hz, 1H) 4.85 (b, |
| | | 3H) 5.29 (d, J = 6.83 Hz, 1H) 6.68 (s, 2H) 7.42 (m, 5H) |
| 86 | [M + 1] = 324.1 | ¹H NMR (400 MHz, METHANOL-D4) □ ppm 2.89 (m, 1H) 2.96 (m, 1H) |
| | | 3.09 (td, J = 12.49, 3.90 Hz, 1H) 3.21 (dt, J = 12.83, 1.20 Hz, 1H) 3.82 (td, |
| | | J = 12.44, 2.64 Hz, 1H) 4.11 (dd, J = 12.88, 3.32 Hz, 1H) 4.19 (ddd, J = 10.93, |
| | | 6.25, 2.54 Hz, 1H) 4.85 (b, 3H) 5.22 (d, J = 6.25 Hz, 1H) 6.67 (s, 2H) |
| | | 6.78 (m, 2H) 7.38 (m, 5H) |
| 87 | [M + 1] = 318.1 | ¹H NMR (400 MHz, METHANOL-D4) □ ppm 2.14 (s, 3H) 3.05 (m, 2H) |
| | | 3.18 (m, 2H) 3.81 (td, J = 12.49, 2.54 Hz, 1H) 4.10 (dd, J = 12.88, 3.51 Hz, 1H) |
| | | 4.17 (ddd, J = 11.13, 5.17, 2.24 Hz, 1H) 4.85 (b, 3H) 5.46 (d, J = 5.27 Hz, 1H) |
| | | 6.69 (m, 4H) 7.18 (d, J = 8.00 Hz, 1H) 7.36 (m, 5H) |
| 88 | [M + 1] = 372.1 | ¹H NMR (400 MHz, METHANOL-D4) □ ppm 3.08 (m, 3H) 3.22 (m, 1H) |
| | | 3.83 (td, J = 12.49, 2.54 Hz, 1H) 4.12 (dd, J = 12.88, 3.12 Hz, 1H) 4.21 (ddd, |
| | | J = 9.61, 5.61, 3.90 Hz, 1H) 4.85 (b, 3H) 5.56 (d, J = 5.47 Hz, 1H) 6.68 (s, 2H) |
| | | 7.17 (m, 2H) 7.39 (m, 5H) 7.54 (d, J = 8.20 Hz, 1H) |
| 89 | [M + 1] = 338.0 | ¹H NMR (400 MHz, METHANOL-D4) □ ppm 3.06 (m, 3H) 3.22 (m, 1H) |
| | | 3.81 (td, J = 12.49, 2.54 Hz, 1H) 4.16 (m, 2H) 4.85 (b, 3H) 5.48 (d, J = 5.47 Hz, |
| | | 1H) 6.68 (s, 2H) 6.90 (m, 2H) 7.37 (m, 6H) |
| 90 | [M + 1] = 322.1 | ¹H NMR (400 MHz, METHANOL-D4) d ppm 3.03 (m, 2H) 3.11 (dd, J = 12.20, |
| | | 4.00 Hz, 1H) 3.22 (dt, J = 12.93, 1.24 Hz, 1H) 3.82 (td, J = 12.54, 2.64 Hz, 1H) |
| | | 4.11 (dd, J = 13.08, 3.12 Hz, 1H) 4.17 (ddd, J = 9.66, 5.66, 3.81 Hz, 1H) |
| | | 4.86 (b, 3H) 5.39 (d, J = 5.47 Hz, 1H) 6.67 (s, 2H) 6.88 (m, 2H) 6.96 (m, 1H) |
| | | 7.37 (m, 5H) |
| 91 | [M + 1] = 402.0 | ¹H NMR (400 MHz, METHANOL-D4) □ ppm 2.87 (d, J = 7.03 Hz, 2H) |
| | | 3.04 (td, J = 12.59, 3.90 Hz, 1H) 3.16 (s, 1H) 3.80 (m, 4H) 4.05 (dd, J = 12.98, |
| | | 3.03 Hz, 1H) 4.24 (q, J = 6.64 Hz, 1H) 4.85 (b, 3H) 5.52 (d, J = 6.64 Hz, 1H) |
| | | 6.68 (s, 2H) 7.09 (s, 1H) 7.35 (m, 3H) 7.42 (m, 2H) |
| 92 | [M + 1] = 334.1 | ¹H NMR (400 MHz, METHANOL-D4) □ ppm 2.87 (d, J = 7.42 Hz, 2H) |
| | | 3.00 (dd, J = 12.01, 3.81 Hz, 1H) 3.06 (m, 1H) 3.76 (td, J = 12.15, 2.83 Hz, 1H) |
| | | 3.90 (s, 3H) 4.07 (m, 2H) 4.84 (b, 3H) 5.30 (d, J = 5.86 Hz, 1H) 6.65 (s, 2H) |
| | | 6.78 (m, 2H) 6.91 (m, 1H) 7.35 (m, 3H) 7.43 (m, 2H) |
| 93 | [M + 1] = 368.0 | ¹H NMR (400 MHz, METHANOL-D4) □ ppm 3.02 (m, 2H) 3.10 (m, 1H) |
| | | 3.17 (m, 1H) 3.77 (m, 1H) 3.85 (s, 3H) 4.11 (t, J = 9.66 Hz, 2H) 4.84 (b, 3H) |
| | | 5.32 (d, J = 5.47 Hz, 1H) 6.68 (s, 2H) 6.90 (s, 1H) 7.07 (s, 1H) 7.36 (m, |
| | | 5H) |
| 94 | [M + 1] = 378.0 | ¹H NMR (400 MHz, METHANOL-D4) □ ppm 3.07 (m, 3H) 3.20 (m, 1H) |
| | | 3.78 (dd, J = 12.79, 2.64 Hz, 1H) 3.83 (s, 3H) 4.12 (m, 2H) 4.85 (b, 3H) |
| | | 5.29 (d, J = 5.27 Hz, 1H) 6.67 (m, 3H) 6.83 (dd, J = 8.59, 2.34 Hz, 1H) |
| | | 7.06 (d, J = 2.15 Hz, 1H) 7.35 (m, 5H) |
| 95 | [M + 1] = 441.9 | ¹H NMR (400 MHz, METHANOL-D4) □ ppm 2.61 (m, 1H) 2.82 (m, 1H) |
| | | 3.00 (m, 1H) 3.12 (m, 1H) 3.74 (m, 1H) 3.92 (m, 1H) 4.37 (m, 1H) |
| | | 4.84 (b, 3H) 5.49 (d, J = 7.81 Hz, 1H) 6.69 (s, 2H) 7.44 (m, 3H) 7.53 (m, 2H) |
| 96 | [M + 1] = 334.1 | ¹H NMR (400 MHz, METHANOL-D4) □ ppm 3.05 (m, 2H) 3.17 (m, 2H) |
| | | 3.67 (s, 3H) 3.82 (td, J = 12.59, 2.54 Hz, 1H) 4.10 (dd, J = 12.88, 3.12 Hz, 1H) |
| | | 4.17 (ddd, J = 11.08, 5.12, 2.15 Hz, 1H) 4.85 (b, 3H) 5.33 (d, J = 5.08 Hz, |
| | | 1H) 6.63 (dd, J = 9.18, 2.93 Hz, 1H) 6.67 (s, 2H) 6.80 (d, J = 8.98 Hz, 1H) |
| | | 6.91 (d, J = 3.12 Hz, 1H) 7.35 (m, 5H) |
| 97 | [M + 1] = 334.1 | ¹H NMR (400 MHz, METHANOL-D4) □ ppm 3.05 (m, 2H) 3.20 (m, 2H) |
| | | 3.61 (s, 3H) 3.82 (td, J = 12.49, 2.54 Hz, 1H) 4.11 (dd, J = 12.79, 3.22 Hz, 1H) |
| | | 4.17 (ddd, J = 11.13, 5.08, 2.34 Hz, 1H) 4.85 (b, 3H) 5.44 (d, J = 5.27 Hz, |
| | | 1H) 6.43 (td, J = 7.91, 2.73 Hz, 2H) 6.68 (s, 2H) 7.20 (d, J = 8.59 Hz, 1H) |
| | | 7.37 (m, 5H) |
| 98 | [M + 1] = 372.0 | ¹H NMR (400 MHz, METHANOL-D4) □ ppm 2.71 (d, J = 1.56 Hz, 1H) |
| | | 2.82 (m, 1H) 3.02 (td, J = 12.59, 3.90 Hz, 1H) 3.17 (m, 1H) 3.80 (td, J = 12.59, |
| | | 2.54 Hz, 1H) 3.98 (dd, J = 12.88, 3.71 Hz, 1H) 4.37 (ddd, J = 11.22, 7.22, |
| | | 2.44 Hz, 1H) 4.86 (b, 3H) 5.45 (d, J = 7.22 Hz, 1H) 6.67 (s, 2H) 7.40 (m, 5H) |
| | | 7.50 (m, 2H) |
| 99 | [M + 1] = 368.1 | ¹H NMR (400 MHz, METHANOL-D4) □ ppm 3.08 (m, 3H) 3.19 (m, 1H) |
| | | 3.82 (td, J = 12.43, 2.63 Hz, 1H) 3.91 (s, 3H) 4.15 (m, 2H) 4.85 (b, 3H) |
| | | 5.43 (d, J = 5.46 Hz, 1H) 6.68 (s, 2H) 6.89 (d, J = 8.38 Hz, 1H) 7.01 (m, 1H) |
| | | 7.16 (d, J = 1.75 Hz, 1H) 7.37 (m, 5H) |
| 100 | [M + 1] = 368.0 | ¹H NMR (400 MHz, METHANOL-D4) □ ppm 3.07 (m, 2H) 3.21 (m, 2H) |
| | | 3.77 (m, 1H) 3.85 (s, 3H) 4.10 (d, J = 12.48 Hz, 2H) 4.84 (b, 3H) 5.31 (d, |
| | | J = 4.48 Hz, 1H) 6.68 (d, J = 0.97 Hz, 2H) 6.74 (s, 2H) 6.97 (m, 1H) 7.32 (m, |
| | | 3H) 7.44 (m, 1H) |
| 101 | [M + 1] = 348.1 | ¹H NMR (400 MHz, METHANOL-D4) □ ppm 2.21 (s, 3H) 3.12 (m, 2H) |
| | | 3.23 (m, 1H) 3.78 (m, 2H) 3.83 (s, 3H) 4.10 (m, 2H) 4.85 (b, 3H) 5.26 (d, |
| | | J = 4.48 Hz, 1H) 6.53 (dd, J = 8.19, 1.36 Hz, 1H) 6.65 (d, J = 7.99 Hz, 1H) |
| | | 6.68 (s, 2H) 6.78 (d, J = 1.75 Hz, 1H) 7.30 (m, 3H) 7.45 (d, J = 1.36 Hz, 1H) |
| 102 | [M + 1] = 356.0 | ¹H NMR (400 MHz, METHANOL-D4) □ ppm 3.07 (td, J = 12.57, 4.09 Hz, 2H) |
| | | 3.21 (d, J = 12.67 Hz, 2H) 3.80 (m, 1H) 4.14 (m, 2H) 4.85 (b, 3H) 5.44 (d, |
| | | J = 4.87 Hz, 1H) 6.68 (s, 2H) 6.88 (m, 2H) 7.20 (m, 1H) 7.34 (m, 3H) |
| | | 7.44 (s, 1H) |
| 103 | MS(APCI) | ¹H NMR (400 MHz, METHANOL-D4) d ppm 3.1 (m, 2H) 3.2 (m, 2H) |
| | M + 1 = 335.1 | 3.7 (ddd, J = 12.9, 11.5, 2.8 Hz, 1H) 3.8 (s, 3H) 4.0 (ddd, J = 12.7, 3.8, 1.6 Hz, 1H) |
| | | 4.2 (ddd, J = 10.2, 4.2, 3.1 Hz, 1H) 5.3 (d, J = 4.2 Hz, 1H) 6.7 (s, 2H) |
| | | 6.7 (m, 2H) 7.0 (dd, J = 1.6, 0.6 Hz, 1H) 7.3 (ddd, J = 7.6, 4.9, 1.2 Hz, 1H) 7.5 (d, |
| | | J = 8.0 Hz, 1H) 7.8 (td, J = 7.7, 1.7 Hz, 1H) 8.5 (ddd, J = 4.9, 1.7, 0.9 Hz, 1H) |
| 104 | MS(APCI) | ¹H NMR (400 MHz, METHANOL-D4) d ppm 3.1 (m, 4H) 3.7 (ddd, J = 12.8, |
| | M + 1 = 323.0 | 11.6, 2.9 Hz, 1H) 4.1 (ddd, J = 12.9, 3.7, 1.2 Hz, 1H) 4.2 (dt, J = 8.4, 4.8 Hz, |
| | | 1H) 5.4 (d, J = 4.7 Hz, 1H) 6.7 (s, 2H) 6.8 (t, J = 8.8 Hz, 1H) 7.0 (ddd, J = 8.8, |
| | | 2.5, 1.6 Hz, 1H) 7.2 (dd, J = 11.0, 2.5 Hz, 1H) 7.4 (ddd, J = 7.6, 4.9, 1.1 Hz, 1H) |
| | | 7.5 (dt, J = 7.8, 1.0 Hz, 1H) 7.8 (td, J = 7.8, 1.8 Hz, 1H) 8.6 (ddd, J = 4.9, |
| | | 1.6, 0.9 Hz, 1H) |
| 105 | MS(APCI) | ¹H NMR (400 MHz, METHANOL-D4) d ppm 3.1 (m, 1H) 3.2 (m, 1H) 3.2 (m, |
| | M + 1 = 323.0 | 2H) 3.7 (m, 1H) 4.0 (ddd, J = 12.8, 3.8, 0.9 Hz, 1H) 4.2 (m, J = 13.2, 7.3, 7.1, |
| | | 4.5 Hz, 1H) 5.4 (d, J = 4.5 Hz, 1H) 6.6 (s, 2H) 6.8 (dd, J = 9.2, 4.9 Hz, 1H) |
| | | 6.8 (ddd, J = 9.2, 7.9, 3.0 Hz, 1H) 7.2 (dd, J = 8.2, 2.9 Hz, 1H) 7.3 (ddd, |
| | | J = 7.6, 4.9, 1.1 Hz, 1H) 7.5 (dt, J = 7.9, 0.9 Hz, 1H) 7.8 (td, J = 7.8, 1.7 Hz, 1H) |
| | | 8.5 (ddd, J = 4.9, 1.7, 1.0 Hz, 1H) |
| 106 | MS(APCI) | ¹H NMR (400 MHz, METHANOL-D4) d ppm 3.1 (td, J = 12.4, 3.8 Hz, 1H) |
| | M + 1 = 335.1 | 3.2 (m, 1H) 3.2 (m, 2H) 3.7 (m, 4H) 4.0 (ddd, J = 12.7, 4.0, 1.1 Hz, 1H) 4.2 (dt, |
| | | J = 8.8, 4.5 Hz, 1H) 5.3 (d, J = 4.4 Hz, 1H) 6.6 (dd, J = 9.1, 2.9 Hz, 1H) 6.6 (s, |
| | | 2H) 6.7 (m, 1H) 6.9 (d, J = 2.8 Hz, 1H) 7.3 (ddd, J = 7.6, 4.9, 1.1 Hz, 1H) |
| | | 7.5 (dt, J = 7.9, 0.9 Hz, 1H) 7.8 (td, J = 7.7, 1.9 Hz, 1H) 8.5 (ddd, J = 4.9, 1.7, 0.9 Hz, |
| | | 1H) |
| 107 | MS(APCI) | ¹H NMR (400 MHz, METHANOL-D4) d ppm 3.2 (m, 3H) 3.6 (m, 1H) 3.7 (td, |
| | M + 1 = 334.1 | J = 12.6, 3.4 Hz, 1H) 3.9 (s, 3H) 4.1 (m, 2H) 5.2 (d, J = 6.2 Hz, 1H) 6.7 (m, 2H) |
| | | 7.0 (d, J = 2.0 Hz, 1H) 7.3 (m, 5H) |
| 108 | MS(APCI) | ¹H NMR (400 MHz, METHANOL-D4) d ppm 3.2 (m, 3H) 3.6 (m, 1H) 3.7 (td, |
| | M + 1 = 334.1 | J = 12.6, 3.4 Hz, 1H) 3.9 (s, 3H) 4.1 (m, 2H) 5.2 (d, J = 6.2 Hz, 1H) 6.7 (m, 2H) |
| | | 7.0 (d, J = 2.0 Hz, 1H) 7.3 (m, 5H) |
| 109 | MS(APCI) | ¹H NMR (400 MHz, METHANOL-D4) d ppm 2.9 (d, J = 6.6 Hz, 1H) 2.9 (s, 1H) |
| | M + 1 = 334.1 | 3.0 (m, 1H) 3.1 (m, 1H) 3.7 (m, 1H) 3.8 (s, 3H) 4.0 (m, 2H) 5.2 (d, |
| | | J = 5.4 Hz, 1H) 6.7 (dd, J = 8.7, 2.3 Hz, 1H) 6.7 (m, 1H) 6.9 (d, J = 2.4 Hz, 1H) |
| | | 7.3 (m, 5H) |

### EXAMPLE 110

The compounds of Examples 37-74 and 79-109 were tested as follows for there NET and SERT binding activity.

hNET Receptor Binding:

Cell pastes of HEK-293 cells transfected with a human norepinephrine transporter cDNA were prepared. The cell pastes were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 seconds. Aliquots of membranes (5 mg/ml protein) were stored in liquid nitrogen until used.

The binding assay was set up in Beckman deep-well polypropylene plates with a total volume of 250 μl containing: drug (10⁻⁵M to 10⁻¹²M), cell membranes, and 50 μM [¹²⁵I]-RTI-55 (Perkin Elmer, NEX-272; specific activity 2200 Ci/mmol). The reaction was incubated by gentle agitation for 90 minutes at room temperature and was terminated by filtration through Whatman GF/C filter plates using a Brandel 96-well plate harvester. Scintillation fluid (100 μl) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM desipramine.

Excel and GraphPad Prism software were used for data calculation and analysis. IC₅₀ values were converted to Kᵢ values using the Cheng-Prusoff equation. The Kᵢ values (nM) for the hNET are reported below in Table 1.

hSERT Receptor Binding

Cell pastes of HEK-293 cells transfected with a human serotonin transporter cDNA were prepared. The cell pastes were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at Setting 7 for 30 seconds. Aliquots of membranes (−2.5 mg/ml protein) were stored in liquid nitrogen until used.

Assays were set up in FlashPlates pre-coated with 0.1% PEI in a total volume of 250 μl containing: drug (10⁻⁵M to 10⁻¹²M), cell membranes, and 50 μM [¹²⁵I]-RTI-55 (Perkin Elmer, NEX-272; specific activity 2200 Ci/mmol). The reaction was incubated and gently agitated for 90 minutes at room temperature, and terminated by removal of assay volume. Plates were covered, and bound [¹²⁵I]-RTI-55 was determined using a Wallac Trilux Beta Plate Counter. Test compounds were run in duplicate, and specific binding was defined as the difference between binding in the presence and absence of 10 μM citalopram. Excel and GraphPad Prism software were used for data calculation and analysis. IC₅₀ values were converted to Kᵢ values using the Cheng-Prusoff equation. The Kᵢ values (nM) for the hSERT are reported below in Table 1.

TABLE 1

| | NET Kᵢ | SERT Kᵢ | | NET Kᵢ | SERT Kᵢ | | NET Kᵢ | SERT Kᵢ |
|---|---|---|---|---|---|---|---|---|
| Ex. No. | (nM) | (nM) | Ex. No. | (nM) | (nM) | Ex. No. | (nM) | (nM) |
| 37 | 13.1 | 52.2 | 60 | 1.91 | 313.6 | 87 | 18.0 | 184.2 |
| 38 | 7.6 | 38.7 | 61 | 20.14 | 38.05 | 88 | 420.0 | 759.9 |
| 39 | 3.77 | 142.5 | 62 | 15.27 | 152.8 | 89 | 20.3 | 171.0 |
| 40 | 4.3 | 157.8 | 63 | 3.21 | 124.4 | 90 | 4.4 | 127.2 |
| 41 | 7.3 | 125.8 | 64 | 13.83 | 510.5 | 91 | 228.1 | 26.7 |
| 42 | 11.4 | 69.2 | 65 | 15.69 | 511.3 | 92 | 86.8 | 71.7 |
| 43 | 1696.0 | 4.7 | 66 | 19.88 | 1035 | 93 | 164.2 | 70.0 |
| 44 | 33.3 | 74.8 | 67 | 390.4 | 35.55 | 94 | 12.1 | 22.3 |
| 45 | 10.5 | 30.0 | 68 | 20.05 | 408 | 95 | 587.0 | 39.6 |
| 46 | 15.1 | 222.0 | 69 | 7.92 | 687.1 | 96 | 25.7 | 47.0 |
| 47 | 6.9 | 967.2 | 70 | 11.85 | 110.6 | 97 | 42.5 | 133.9 |
| 48 | 16.5 | 80.0 | 71 | 28.55 | 77.68 | 98 | 755.2 | 49.6 |
| 49 | 6.8 | 193.7 | 72 | 91.65 | 78.9 | 99 | 187.9 | 35.8 |
| 50 | 8.0 | 1068.0 | 73 | 75.74 | 14 | 100 | 32.97 | 32.9 |
| 51 | 26.6 | 1036.0 | 74 | 18.63 | 138.3 | 101 | 19.4 | 67.1 |
| 52 | 549 | 193.6 | 79 | 12.6 | 100.2 | 102 | 48.28 | 221.8 |
| 53 | 26.57 | 344.4 | 80 | 4.4 | 127.2 | 103 | 12.8 | 101 |
| 54 | 23.73 | 41.74 | 81 | 7.1 | 26.4 | 104 | 21.1 | 486 |
| 55 | 20.38 | 109 | 82 | 11.9 | 12.1 | 105 | 3.5 | 621 |
| 56 | 13.59 | 151.5 | 83 | 8.9 | 222.5 | 106 | 13.8 | 216 |
| 57 | 19.67 | 544.5 | 84 | 10.1 | 436.8 | 107 | 182.6 | 10.3 |
| 58 | 42.89 | 474.3 | 85 | 10.9 | 385.3 | 108 | 2022.0 | 14.6 |
| 59 | 10.35 | 524 | 86 | 14.3 | 652.4 | 109 | 97.9 | 0.92 |

### EXAMPLE 111

### (2S)-2-[(1S)-(4-chloro-2-methoxyphenoxy)(phenyl)methyl]morpholine benzene sulfonate

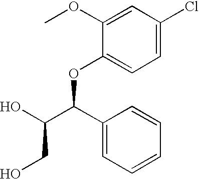

### (2R,3S)-3-(4-chloro-2-methoxyphenoxy)-3-phenylpropane-1,2-diol

Sodium hydroxide (1.44 g, 36 mmol) was dissolved in water (75 ml). 4-Chloro-2-methoxyphenol (12 g, 76 mmol) was added and the mixture was warmed to 70° C. To this solution was added (2R,3R)-phenylglycidol (5.4 g, 36 mmol). The mixture was stirred at 70° C. for 2.5 hours, then cooled to room temperature and poured into 5% aqueous NaOH (100 ml). The solution was extracted three times with 100 ml of CH₂Cl₂. The combined organic layers were washed with 5% aqueous NaOH (100 ml) and brine (100 ml) then dried over Na₂SO₄. Filtration and concentration under reduced pressure provided an oily solid that was suspended in toluene (75 ml) and stirred for 5 minutes at 60° C. The suspension was cooled in an ice bath and then filtered, providing (2R,3S)-3-(4-chloro-2-methoxyphenoxy)-3-phenylpropane-1,2-diol (8.4 g) as a white solid. ¹H NMR (400 MHz, CHLOROFORM-D) δ ppm 1.6 (s, 2 H) 2.8 (dd, J=9.4, 3.7 Hz, 1H) 3.0 (ddd, J=7.4, 2.0, 1.9 Hz, 1H) 3.7 (m, 1H) 3.9 (s, 3H) 3.9 (m, 2H) 5.2 (d, J=4.3 Hz, 1H) 6.5 (d, J=8.6 Hz, 1H) 6.7 (dd, J=8.7, 2.4 Hz, 1H) 6.9 (d, J=2.3 Hz, 1H) 7.3 (m, 5H)

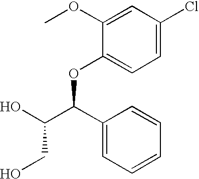

### (1S,2S)-3-amino-1-(4-chloro-2-methoxyphenoxy)-1-phenylpropan-2-ol

(2R,3S)-3-(4-chloro-2-methoxyphenoxy)-3-phenylpropane-1,2-diol (23 g, 74 mmol) was suspended in CH₂Cl₂ (250 ml). Triethylamine (12.5 ml, 89 mmol) was added and the slightly cloudy solution was cooled to −30° C. (internal). A solution of chlorotrimethylsilane (9.9 ml, 78 mmol) in CH₂Cl₂ (40 ml) was added dropwise over 45 minutes. The mixture was stirred at −30° C. for an additional 10 minutes, at which time no starting diol remained by TLC (thin-layer chromatography), to yield the silyl ether ((1S,2R)-1-(4-chloro-2-methoxyphenoxy)-1-phenyl-3-[(trimethylsilyl)oxy]propan-2-ol).

To the cold solution of silylether was added triethylamine (12.5 ml, 89 mmol). A solution of methanesulfonyl chloride (6.9 ml, 89 mmol) in CH₂Cl₂ (30 ml) was then added dropwise over 15 minutes. The mixture was stirred at −30° C. for an additional 45 minutes, at which time no starting silylether remained by TLC, to yield the mesylate ((1R,2S)-2-(4-chloro-2-methoxyphenoxy)-2-phenyl-1-{[(trimethylsilyl)oxy]methyl}ethyl methanesulfonate).

To the cold solution of mesylate was added 1 M HCl (75 ml). The mixture was warmed to room temperature and stirred for an additional 1 hour. The organic layer was separated and washed with 10% aqueous NaHCO₃ and then concentrated under reduced pressure to an oil ((1R,2S)-2-(4-chloro-2-methoxyphenoxy)-1-(hydroxymethyl)-2-phenylethyl methanesulfonate).

To a toluene (150 ml) solution of the oil to yield ((2R)-2-[(S)-(4-chloro-2-methoxyphenoxy)(phenyl)methyl]oxirane) was added tetrabutylammonium chloride (1 g, 3.7 mmol), water (50 ml) and 50% aqueous NaOH (20 g, 250 mmol). The biphasic mixture was stirred rapidly at room temperature for 18 hours. The organic layer was separated and washed with brine. The solution was concentrated under reduced pressure to one-quarter of its original volume. MeOH (300 ml) was added and the solution was again concentrated under reduced pressure to one-quarter of its original volume.

The solution above was diluted with MeOH (250 ml) and treated with concentrated NH₄OH (250 ml). The heterogenous mixture was warmed to 40° C. and stirred at that temperature for 3 hours during which time the mixture became homogenous. The solution was cooled to room temperature and stirred for an additional 18 hours. CH₂Cl₂ (200 ml) was added and the layers separated. The aqueous layer was extracted twice with 300 ml of CH₂Cl₂. The combined organic layers were concentrated under reduced pressure to a paste that was suspended in ether (300 ml). The suspension was treated with aqueous HCl (500 ml, pH 4) and stirred rapidly at room temperature until all solids dissolved. The layers were separated and the aqueous layer was made basic with 5% aqueous NaOH. The resulting precipitate was extracted twice into 300 ml of CH₂Cl₂. The organic solution was concentrated under reduced pressure to a gelatinous solid that was suspended in toluene (150 ml) and reconcentrated to provide (1S,2S)-3-amino-1-(4-chloro-2-methoxyphenoxy)-1-phenylpropan-2-ol (20 g) as a white solid. ¹H NMR (400 MHz, CHLOROFORM-D) δ ppm 2.7 (dd, J=13.0, 6.7 Hz, 1H) 2.8 (m, 1H) 3.9 (s, 3H) 4.0 (td, J=6.8, 3.7 Hz, 1 H) 4.8 (d, J=7.2 Hz, 1H) 6.5 (d, J=8.6 Hz, 1H) 6.7 (dd, J=8.6, 2.5 Hz, 1H) 6.8 (d, J=2.3 Hz, 1H) 7.3 (m, 5H). MS (APCI) 308.1 (M+1).

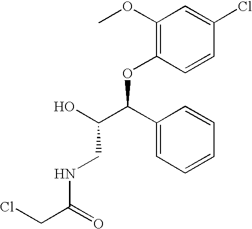

### 2-chloro-N-[(2S,3S)-3-(4-chloro-2-methoxyphenoxy)-2-hydroxy-3-phenylpropyl]acetamide

(1S,2S)-3-amino-1-(4-chloro-2-methoxyphenoxy)-1-phenylpropan-2-ol (20 g, 65 mmol) was suspended in toluene (200 ml). Aqueous Na₂CO₃ solution (11 g in 150 ml water) was added to the mixture. The rapidly stirred mixture was cooled in an ice bath. A solution of chloroacetylchloride (5.4 ml, 67 mmol) in toluene (30 ml) was added dropwise over 10-15 minutes. The mixture was stirred for an additional 10 minutes at 0° C., then warmed to room temperature and stirred for an additional 1.5 hours. The layers were separated and the organic layer was washed with water and brine. The combined aqueous layers were washed with toluene. The combined organic layers were dried over Na₂SO₄, filtered and concentrated under reduced pressure to provide 2-chloro-N-[(2S,3S)-3-(4-chloro-2-methoxyphenoxy)-2-hydroxy-3-phenylpropyl]acetamide as a thick oil (25 g). ¹H NMR (400 MHz, CHLOROFORM-D) δ ppm 3.2 (ddd, J=13.8, 6.9, 5.3 Hz, 1H) 3.4 (ddd, J=13.8, 5.8, 3.9 Hz, 1H) 3.9 (s, 3H) 4.0 (s, 2H) 4.1 (m, 1H) 4.7 (d, J=7.8 Hz, 1H) 6.5 (d, J=8.6 Hz, 1H) 6.7 (dd, J=8.5, 2.4 Hz, 1H) 6.9 (d, J=2.3 Hz, 1H) 7.0 (m, 1H) 7.4 (m, 5H). MS (APCI) 420.0(M+36(HCl) 382.1 (M−2).

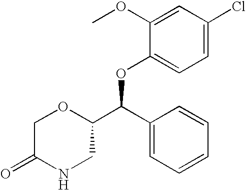

### (6S)-6-[(S)-(4-chloro-2-methoxyphenoxy)(phenyl)methyl]morpholin-3-one

2-Chloro-N-[(2S,3S)-3-(4-chloro-2-methoxyphenoxy)-2-hydroxy-3-phenylpropyl]acetamide (25 g, 65 mmol) from above was dissolved in isopropanol (200 ml). To this was added a solution of potassium tert-butoxide (15 g, 130 mmol) isopropanol (200 ml) dropwise over 1 hour. The mixture was stirred at room temperature for an additional 1.5 hours then acidified with 10% aqueous HCl. The solution was concentrated under reduced pressure and the residue partitioned between water 250 ml and 1:1 EtOAc:CH₂Cl₂ (500 ml). The aqueous layer was extracted with EtOAc (200 ml) and the combined organics were dried over Na₂SO₄, filtered and concentrated under reduced pressure to provide (6S)-6-[(S)-(4-chloro-2-methoxyphenoxy)(phenyl)methyl]morpholin-3-one as a thick oil (22 g). ¹H NMR (400 MHz, CHLOROFORM-D) δ ppm 3.0 (dt, J=11.8, 3.5 Hz, 1H) 3.3 (m, 1H) 3.8 (s, 3H) 4.2 (ddd, J=10.4, 6.4, 3.2 Hz, 1H) 4.3 (d, J=17.0 Hz, 1H) 4.4 (m, 1H) 5.2 (d, J=6.2 Hz, 1H) 6.3 (s, 1H) 6.7 (m, 2H) 6.8 (d, J=2.1 Hz, 1H) 7.3 (m, 5H). MS (APCI) 348.1 (M+1).

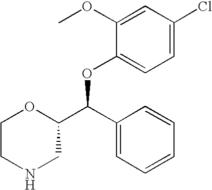

### (2S)-2-[(S)-(4-chloro-2-methoxyphenoxy)(phenyl)methyl]morpholine

(6S)-6-[(S)-(4-Chloro-2-methoxyphenoxy)(phenyl)methyl]morpholin-3-one (1.7 g, 4.9 mmol) prepared as above was dissolved in toluene (75 ml). To this was added a toluene solution of Red-AI (sodium bis(2-methoxyethoxy)aluminum hydride, Aldrich) (4.5 ml 65% solution diluted to 15 ml, 14.7 mmol) dropwise over 15 minutes. The mixture was stirred at room temperature for 2 hours then quenched with 5% aqueous NaOH (15 ml). The layers were separated and the aqueous washed with toluene (50 ml). The combined organics were dried over Na₂SO₄, filtered and concentrated under reduced pressure. The residue was purified by silica gel chromatography eluting with 5%-15% isopropanol in CH₂Cl₂, providing (2S)-2-[(S)-(4-chloro-2-methoxyphenoxy)(phenyl)methyl]morpholine (1.13 g) as a clear viscous oil. ¹H NMR (400 MHz, CHLOROFORM-D) δ ppm 2.0 (s, 2H) 2.7 (m, 2H) 2.9 (m, 2H) 3.7 (td, J=11.2, 3.2 Hz, 1 H) 3.8 (s, 3H) 4.0 (m, 2H) 5.1 (d, J=6.2 Hz, 1H) 6.6 (m, 2H) 6.8 (d, J=1.4 Hz, 1H) 7.3 (m, 5H). MS (APCI) 334.1 (M+1).

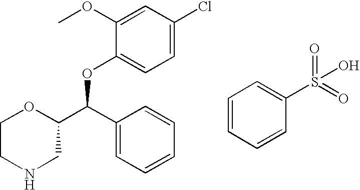

### (2S)-2-[(1S)-(4-chloro-2-methoxyphenoxy)(phenyl)methyl]morpholine benzene sulfonate

(2S)-2-[(S)-(4-Chloro-2-methoxyphenoxy)(phenyl)methyl]morpholine (7 g, 21 mmol) prepared as above was dissolved in isopropanol (50 ml), and then diluted with tert-butylmethylether (100 ml). A isopropanol solution of benzenesulfonic acid (3.5 g, 22 mmol, 20 ml) was then added and the mixture stirred at room temperature. The resulting precipitate was filtered and recrystallized from acetonitrile to provide (2S)-2-[(1S)-(4-chloro-2-methoxyphenoxy)(phenyl)methyl]morpholine benzene sulfonate (6.25 g) as fine needles. ¹H NMR (400 MHz, CHLOROFORM-D) 8 ppm 2.0 (s, 2H) 2.7 (m, 2H) 2.9 (m, 2H) 3.7 (td, J=11.2, 3.2 Hz, 1H) 3.8 (s, 3H) 4.0 (m, 2H) 5.1 (d, J=6.2 Hz, 1H) 6.6 (m, 2H) 6.8 (d, J=1.4 Hz, 1H) 7.3 (m, 5H). MS (APCI) 334.1 (M+1).

### EXAMPLE 112

### (2S)-2-[(1S)-(4-chloro-2-methoxyphenoxy) (phenyl)methyl]morpholine fumarate

(2S)-2-[(1S)(4-Chloro-2-methoxy-phenoxy)-phenyl-methyl]-morpholine-4-carboxylic acid tert-butyl ester was prepared in a manner analogous to that used in the preparation of (2S)-2-[(1S)-(2-chloro-4-fluorophenoxy)-(3-fluorophenyl)methyl]morpholine-4-carboxylic acid tert-butyl ester in the synthesis of Example 38. (2S)-2-[(1S)(4-Chloro-2-methoxy-phenoxy)-phenyl-methyl]-morpholine-4-carboxylic acid tert-butyl ester (0.09 g, 0.21 mmol) was taken up in 5 ml dichloromethane, cooled to 0° C., and 2 ml trifluoroacetic acid (TFA) was added. The ice bath was removed, and the reaction mixture was stirred at room temperature for 1 hour. The solvent and acid were removed under reduced pressure. To the residual oil was added 10 ml H₂O and 10 ml CH₂Cl₂. The biphasic mixture was shaken, and the aqueous layer collected. The pH value of the mixture was adjusted to 13 by adding 1-2 ml 1.0 M NaOH solution. The aqueous phase was extracted using 10 ml CH₂Cl₂. The organic phase was washed with 10 ml H₂O and dried over Na₂SO₄. The solvent was removed under reduced pressure providing 0.068 g (0.20 mmol) 2-[(4-Chloro-2-methoxy-phenoxy)-phenyl-methyl]-morpholine as an oil. The 2-[(4-Chloro-2-methoxy-phenoxy)-phenyl-methyl]-morpholine was then dissolved in 1 ml acetone. The resulting solution was added to a solution of 24 mg (0.20 mmol) fumaric acid in 5 ml acetone and stirred at room temperature. A white gel-like precipitate appeared in about 1 minute. The precipitate was collected by filtration, washed by three times with 1 ml of acetone, and dried under vacuum to give 89 mg (0.20 mmol) of (2S)-2-[(1S)-(4-chloro-2-methoxyphenoxy) (phenyl)methyl]morpholine fumarate salt as a white solid (MP=135-139° C.).

### EXAMPLE 113

### (2S)-2-[(S)-(4-chloro-2-methoxyphenoxy)(phenyl)methyl]morpholine besylate

Approximately 146 mg of benzenesulfonic acid was added to 309 mg of (2S)-2-[(S)-(4-chloro-2-methoxyphenoxy)(phenyl)methyl]morpholine (as a clear oil). Approximately 2 ml of methanol was added and solution was sonicated for less than 1 minute. The solution was placed under stream of N₂ gas until precipitation was observed. The suspension was then placed a 40° C. vacuum oven for approximately 30 minutes (a vacuum was pulled but pressure was not controlled). Approximately 15 ml of isopropyl alcohol was added and suspension was slurried for approximately 2 hours. A solid was collected on a 0.2 μm polypropylene membrane using vacuum filtration. The solid was dried in 40° C. vacuum oven (approximately 1 hour, vacuum was pulled but pressure was not controlled) to give (2S)-2-[(S)-(4-chloro-2-methoxyphenoxy)(phenyl)methyl]morpholine besylate.

### EXAMPLE 114

### (2S)-2-[(S)-(4-chloro-2-methoxyphenoxy)(phenyl)methyl]morpholine hydrochloride

6.05 mg of concentrated HCl was added to 10.25 mg of (2S)-2-[(S)-(4-chloro-2-methoxyphenoxy)(phenyl)methyl]morpholine in 1 ml MeOH. The solution placed under stream of N₂ gas until solvent had evaporated. A mixture of white solid and gel was observed. Approximately 1 ml of methyl tert-butyl ether and approximately 750 μL of isopropyl alcohol were added and solution was capped and stirred overnight. The solid was recovered on a 0.2 μm filter membrane using vacuum filtration and then dried in a vacuum oven at 40° C. for approximately 1 hour to give (2S)-2-[(S)-(4-chloro-2-methoxyphenoxy)(phenyl)methyl]morpholine hydrochloride.

### EXAMPLE 115

### (2S)-2-[(S)-(4-chloro-2-methoxyphenoxy)(Phenyl)methyl]morpholine camsylate

800 μL of (2S)-2-[(S)-(4-chloro-2-methoxyphenoxy)(phenyl)methyl]morpholine in MeOH (concentration=10.25 mg/ml) was added to 5.6 mg of camphorsulfonic acid). The solution was placed under stream of N₂ gas until solvent had evaporated. A clear gel remained. Approximately 1 ml of methyl tert-butyl ether and 200 μL of isopropyl alcohol (IPA) was added and solution was sonicated for about 1 minute. A white precipitate was observed. 400 μL more iPA was added and solution was stirred overnight. The solution was placed under stream of N₂ gas until solvent had evaporated and resultant solid was dried in a 40° C. vacuum oven for approximately 2 hours to give (2S)-2-[(S)-(4-chloro-2-methoxyphenoxy)(phenyl)methyl]morpholine camsylate.

### EXAMPLE 116

### (2S)-2-[(S)-(4-chloro-2-methoxyphenoxy)(phenyl)methyl]morpholine citrate. (2S)-2-[(S)-(4-chloro-2-methoxyphenoxy)(phenyl)methyl]morpholine L-tartrate, and (2S)-2-[(S)-(4-chloro-2-methoxyphenoxy)(phenyl)methyl]morpholine fumarate

500 μL aliquots of (2S)-2-[(S)-(4-chloro-2-methoxyphenoxy)(phenyl)methyl]morpholine in MeOH (concentration=31.7 mg/ml) were added to 5.7 mg citric acid, 4.5 mg L-tartaric acid, and 3.5 mg fumaric acid. The solutions were then placed under stream of N₂ gas until the solvent had evaporated. Approximately 2 ml of methyl tert-butyl ether was added each vial. Each vial was then subsequently sonicated for about 1 minute. A white precipitate was observed in all vials. The precipitate in the citric acid solution formed a thick gum. The solutions were again placed under stream of N₂ gas until the solvent had evaporated. Solid was observed in vials with L-tartaric acid and fumaric acid. Approximately 1.5 ml dichloromethane (DCM) was pipetted into all vials and solutions were stirred overnight. Solid was observed in all vials. The solids were recovered with 0.2 μm PTFE (polytetrafluoroethylene) membrane filters using vacuum filtration. The solids were then dried in a vacuum oven at 40° C. for approximately 20 minutes to respectively give (2S)-2-[(S)-(4-chloro-2-methoxyphenoxy)(phenyl)methyl]morpholine citrate, (2S)-2-[(S)-(4-chloro-2-methoxyphenoxy)(phenyl)methyl]morpholine L-tartrate, and (2S)-2-[(S)-(4-chloro-2-methoxyphenoxy)(phenyl)methyl]morpholine fumarate.

### EXAMPLE 117

### (2S)-2-[(S)-(4-chloro-2-methoxyphenoxy)(phenyl)methyl]morpholine L-Tartrate, Phosphate, and Citrate

Equimolar aliquots (820 μL, 790 μL, and 850 μL) of (2S)-2-[(S)-(4-chloro-2-methoxyphenoxy)(phenyl)methyl]morpholine in MeOH (concentration=31.7 mg/ml) were added to 7.36 mg phosphoric acid (MW=98),12.15 mg citric acid (MW=192), and 10.25 mg L-tartaric acid (MW=150), respectively. The solutions were placed under streams of N₂ gas until solvents had evaporated. Approximately 1 ml of methyl tert-butyl ether was added to each and solutions were sonicated for about 5 minutes. Approximately 4 ml of isopropyl alcohol was added to each and solutions were sonicated again (<1 minute). The solutions were stirred overnight, uncapped. Precipitate was observed in all vials. The solids were collected from remaining solvents using vacuum filtration and all were observed to deliquesce upon exposure to air.

### EXAMPLE 118

### (2S)-2-[(S)-(4-chloro-2-methoxyphenoxy)(phenyl)methyl]morpholine hydrobromide

880 μL of (2S)-2-[(S)-(4-chloro-2-methoxyphenoxy)(phenyl)methyl]morpholine in MeOH (concentration=31.7 mg/ml) was added to 11.85 mg of concentrated hydrobromic acid. The solution was placed under stream of N₂ gas until solvent had evaporated. Approximately 1 ml of methyl tert-butyl ether was added and solution was placed in hood uncapped overnight to evaporate the solvent. Approximately 2 ml of isopropyl alcohol was added and suspension was stirred overnight, uncovered. The solvent evaporated to give (2S)-2-[(S)-(4-chloro-2-methoxyphenoxy)(phenyl)methyl]morpholine hydrobromide as a white solid.

### EXAMPLE 119

### (2S)-2-[(S)-(4-chloro-2-methoxyphenoxy)(phenyl)methyl]morpholine edisylate

880 μL of (2S)-2-[(S)-(4-chloro-2-methoxyphenoxy)(phenyl)methyl]morpholine in MeOH (concentration=31.7 mg/ml) was added to 13.4 mg of ethane disulfonic acid (MW=190). The solution placed under stream of N₂ gas until solvent had evaporated. Approximately 1 ml of methyl tert-butyl ether was added and solution was sonicated for about 5 minutes. Approximately 4 ml of isopropyl alcohol was added and solution was sonicated again (<1 minute). The solution was stirred overnight, uncapped. The solid was collected from remaining solvent using vacuum filtration. The solid was dried for approximately 20 minutes in a dessicator chamber attached to a vacuum pump to give (2S)-2-[(S)-(4-chloro-2-methoxyphenoxy)(phenyl)methyl]morpholine edisylate.

### EXAMPLE 120

### (2S)-2-[(S)-(4-chloro-2-methoxyphenoxy)(phenyl)methyl]morpholine succinate

830 μL of (2S)-2-[(S)-(4-chloro-2-methoxyphenoxy)(phenyl)methyl]morpholine in MeOH (concentration 31.7 mg/ml) was added to 7.87 mg of succinic acid. The solution placed under stream of N₂ gas until solvent had evaporated. Approximately 1 ml of dichloromethane was added and vial was left uncapped in hood for approximately 48 hours. Solvent had evaporated and white solid remained ((2S)-2-[(S)-(4-chloro-2-methoxyphenoxy)(phenyl)methyl]morpholine succinate).

### EXAMPLE 121

### Powder X-Ray Diffraction (PXRD)

The experimental powder x-ray diffractions of the compounds of Examples 113-116, 118, and 120 were carried out utilizing a Bruker D8 X-ray powder diffractometer with GADDS (General Area Diffraction Detector System) C2 system with a single Goebel mirror configuration. The scans were run with the detector at 15.0 cm. Theta 1, or the collimator, was at 70 and Theta 2, or the detector, was at 17°. The scan axis was 2-omega with a width of 30. At the end of each scan theta 1 is at 100 and theta 2 is at 140. Samples were run for 60 seconds at 40 kV and 40 mA with CuKα radiation (λ=1.5419 Å). Scans were integrated from 6.4° to 41° 2θ. The samples were run in ASC-6 sample holders purchased from Gem Dugout (State College, Pa.). The samples were placed in the cavity in the middle of the sample holder, and flattened with a spatula to be even with the surface of the holder. All analyses were conducted at room temperature (generally 20° C.-30° C.). Scans were evaluated using DiffracPlus software, release 2003, with Eva version 9.0.0.2.

The experimental powder x-ray diffractions of (2S)-2-[(S)-(4-chloro-2-methoxyphenoxy)(phenyl)methyl]morpholine edisylate (Example 119) was carried out utilizing a Rigaku Ultima+diffractometer with CuKα (40 mA, 40 kV, λ=1.5419 Å) radiation. Diffractometer had an IBM-compatible interface and was equipped with 6 position autosampler. Sample was tapped out of vial and pressed onto zero-background silicon in aluminum holder. Holder was purchased from Gem Dugout (State College, Pa.). Sample width was 5 mm. The scans were run using a continuous 9/20 coupled scan: 3.00° to 45.00° in 2θ, scan rate of 1°/min: 1.2 sec/0.04° step. Slits 1 and 11 were at 0.5°, slit III at 0.6°. Samples were stored and run at room temperature. Samples were spun at 40 rpm around vertical axis during data collection. The scan was evaluated using DiffracPlus software, release 2003, with Eva version 9.0.0.2.

Summaries of the angle (2theta) values and intensity values (as a % of the value of the tallest peak) from the spectra are reported below in Table 2 ((2S)-2-[(S)-(4-chloro-2-methoxyphenoxy)(phenyl)methyl]morpholine besylate); Table 3 ((2S)-2-[(S)-(4-chloro-2-methoxyphenoxy)(phenyl)methyl]morpholine hydrochloride); Table 4 ((2S)-2-[(S)-(4-chloro-2-methoxyphenoxy)(phenyl)methyl]morpholine camsylate); Table 5 ((2S)-2-[(S)-(4-chloro-2-methoxyphenoxy)(phenyl)methyl]morpholine citrate); Table 6 ((2S)-2-[(S)-(4-chloro-2-methoxyphenoxy)(phenyl)methyl]morpholine L-tartrate); Table 7 ((2S)-2-[(S)-(4-chloro-2-methoxyphenoxy)(phenyl)methyl]morpholine fumarate); Table 8 ((2S)-2-[(S)-(4-chloro-2-methoxyphenoxy)(phenyl)methyl]morpholine hydrobromide); Table 9 ((2S)-2-[(S)-(4-chloro-2-methoxyphenoxy)(phenyl)methyl]morpholine edisylate); and Table 10 (2S)-2-[(S)-(4-chloro-2-methoxyphenoxy)(phenyl)methyl]morpholine succinate).

TABLE 2

| Angle (2theta) | Intensity % |
|---|---|
| 8.9° | 11.1 |
| 10.8° | 15.8 |
| 12.0° | 14.9 |
| 13.9° | 19.3 |
| 14.3° | 23.8 |
| 15.1° | 14 |
| 16.6° | 59.1 |
| 17.0° | 40.3 |
| 17.8° | 54 |
| 18.9° | 100 |
| 19.4° | 68.4 |
| 19.9° | 42.4 |
| 20.6° | 45.5 |
| 21.5° | 31.5 |
| 22.4° | 71.2 |
| 22.9° | 60.2 |
| 23.9° | 55.1 |
| 25.7° | 44.9 |
| 27.0° | 40.1 |
| 28.5° | 18.6 |
| 31.0° | 22.2 |

TABLE 3

| Angle (2theta) | Intensity % |
|---|---|
| 8.1° | 31.1 |
| 11.9° | 24.3 |
| 13.9° | 17.8 |
| 16.0° | 40.8 |
| 17.1° | 51.6 |
| 19.0° | 27.5 |
| 19.8° | 57.9 |
| 20.1° | 71.3 |
| 20.9° | 100 |
| 23.5° | 58.2 |
| 24.2° | 64.6 |
| 24.7° | 71.7 |
| 25.6° | 55.3 |
| 27.6° | 43.8 |
| 28.9° | 32.9 |
| 30.4° | 22 |
| 31.5° | 24.2 |
| 32.8° | 44.2 |
| 35.7° | 26.7 |
| 37.4° | 18.6 |

TABLE 4

| Angle (2theta) | Intensity % |
|---|---|
| 12.1° | 49.3 |
| 13.6° | 28.6 |
| 15.1° | 64.9 |
| 16.4° | 49.8 |
| 17.5° | 39.1 |
| 18.1° | 100 |
| 18.9° | 36 |
| 19.7° | 45.1 |
| 20.4° | 39.5 |
| 21.2° | 39.4 |
| 22.5° | 44.9 |
| 24.2° | 26.2 |
| 25.7° | 46.9 |
| 27.1° | 29.8 |
| 29.9° | 16.9 |
| 30.8° | 19.4 |
| 35.6° | 21.5 |
| 38.0° | 19 |

TABLE 5

| Angle (2theta) | Intensity % |
|---|---|
| 11.2° | 36.3 |
| 11.7° | 83.5 |
| 12.6° | 40.5 |
| 14.2° | 34.1 |
| 16.7° | 58.2 |
| 17.6° | 49.9 |
| 18.7° | 58.2 |
| 19.7° | 91.9 |
| 20.9° | 52.2 |
| 22.7° | 100 |
| 24.5° | 92.8 |
| 25.9° | 47.9 |
| 28.1° | 37.2 |

TABLE 6

| Angle (2theta) | Intensity % |
|---|---|
| 8.7° | 22.9 |
| 10.5° | 15.3 |
| 12.4° | 26.6 |
| 13.1° | 100 |
| 14.5° | 36.3 |
| 15.9° | 35.4 |
| 16.9° | 22.6 |
| 17.9° | 41.5 |
| 18.4° | 31.3 |
| 19.3° | 36.7 |
| 20.0° | 50.6 |
| 20.9° | 49.1 |
| 21.9° | 62.4 |
| 20.9° | 49.1 |
| 21.9° | 62.4 |
| 22.9° | 73 |
| 23.9° | 45.6 |
| 24.7° | 25.4 |
| 25.6° | 35.4 |
| 26.6° | 30.4 |
| 27.1° | 25.2 |
| 29.3° | 27.2 |
| 31.0° | 23.3 |
| 32.9° | 17.9 |
| 37.3° | 19.4 |

TABLE 7

| Angle (2theta) | Intensity % |
|---|---|
| 12.0° | 45.8 |
| 13.7° | 32 |
| 15.0° | 31.7 |
| 15.7° | 25.7 |
| 18.4° | 58.7 |
| 19.4° | 100 |
| 20.0° | 82.1 |
| 22.2° | 48.9 |
| 23.9° | 81 |
| 25.1° | 34.5 |
| 26.1° | 34.9 |
| 27.4° | 49.4 |
| 35.4° | 24.6 |

TABLE 8

| Angle (2theta) | Intensity % |
|---|---|
| 10.6° | 15.5 |
| 11.9° | 12.8 |
| 13.8° | 20.5 |
| 14.8° | 11.3 |
| 16.8° | 20.2 |
| 17.5° | 27.4 |
| 19.2° | 23.8 |
| 19.7° | 23.6 |
| 20.5° | 42.1 |
| 21.1° | 100 |
| 23.1° | 79.3 |
| 23.8° | 75.3 |
| 25.4° | 63.9 |
| 27.1° | 23.2 |
| 28.3° | 21.2 |
| 28.7° | 23.5 |
| 29.6° | 32.6 |
| 31.5° | 21.6 |
| 33.8° | 29.5 |
| 35.1° | 18.6 |
| 36.0° | 13.6 |
| 38.3° | 14.3 |

TABLE 9

| Angle (2theta) | Intensity % |
|---|---|
| 3.4° | 100 |
| 4.7° | 53.8 |
| 5.2° | 53.3 |
| 6.6° | 21.6 |
| 8.5° | 22.7 |
| 9.5° | 27 |
| 11.8° | 25.4 |
| 13.8° | 30.9 |
| 15.9° | 12.3 |
| 17.0° | 28.7 |
| 18.5° | 57.9 |
| 19.9° | 60.1 |
| 22.1° | 47.3 |
| 23.1° | 30.6 |
| 25.2° | 32.5 |
| 25.9° | 31.4 |
| 26.7° | 21.3 |
| 28.7° | 18.3 |
| 42.4° | 13.9 |

TABLE 10

| Angle (2theta) | Intensity % |
|---|---|
| 11.8° | 59.1 |
| 13.8° | 20.5 |
| 14.8° | 28.9 |
| 15.7° | 14.8 |
| 18.2° | 57.2 |
| 19.4° | 76.5 |
| 20.0° | 77.5 |
| 22.6° | 41 |
| 23.5° | 100 |
| 24.8° | 27.2 |
| 26.0° | 20.8 |
| 24.8° | 27.2 |
| 26.0° | 20.8 |
| 26.7° | 20.4 |
| 27.4° | 47 |
| 28.9° | 20.8 |
| 29.9° | 16.3 |
| 32.3° | 17.4 |
| 33.5° | 13.8 |
| 35.1° | 20 |
| 37.5° | 12.3 |

### EXAMPLE 122

### Differential Scanning Calorimetry

Differential scanning calorimetry (DSC) was carried out on a TA Instruments DSC Q1000 V8.1 Build 261. Samples were prepared by weighing a sample into an aluminum pan which was then covered with a pierced aluminum lid (TA Instruments' part nos. 900786.901 (bottoms) and 900779.901 (top)). The experiment started at ambient temperature and heated the sample at 10° C./minute to 250° C. under a nitrogen gas purge (flow rate was 50 ml/min). Data was analyzed using Universal Analysis 2000 for Windows 95/98/20001NT/Me/XP version 3.8B, Build 3.8.019. The DSC analyses of the campsylate and HCl salts were carried out as for the besylate salt, except the samples were was scanned from ambient temperature to 200° C. The DSC analyses of the HBr, L-tartrate salt, and citrate salts were carried out as for the besylate salt, except the samples were was scanned from ambient temperature to 175° C. The DSC analyses of the succinate, and fumarate salts were carried out as for the besylate salt, except the samples were was scanned from ambient temperature to 150° C. The DSC analysis of the edisylate salt was carried out as for the besylate salt, except the samples were was scanned from ambient temperature to 300° C. The melting point onset (° C.) for the salts and the amount of the material analyzed are reported in Table 11:

TABLE 11

| | | Melting | |
|---|---|---|---|
| | | Peak | |
| | | Onset | Amount |
| # | Name | (° C.) | (mg) |
| 1 | (2S)-2-[(S)-(4-chloro-2- | 180.97 | 2.95 |
| | methoxyphenoxy)(phenyl)methyl]morpholine | | |
| | besylate | | |
| 2 | (2S)-2-[(S)-(4-chloro-2- | 148.11 | 2.18 |
| | methoxyphenoxy)(phenyl)methyl]morpholine | | |
| | hydrochloride | | |
| 3 | (2S)-2-[(S)-(4-chloro-2- | 162.03 | 2.54 |
| | methoxyphenoxy)(phenyl)methyl]morpholine | | |
| | camsylate | | |
| 4 | (2S)-2-[(S)-(4-chloro-2- | 119.29 | 2.66 |
| | methoxyphenoxy)(phenyl)methyl]morpholine | | |
| | citrate | | |
| 5 | (2S)-2-[(S)-(4-chloro-2- | 92.68 | 1.52 |
| | methoxyphenoxy)(phenyl)methyl]morpholine | | |
| | L-tartrate | | |
| 6 | (2S)-2-[(S)-(4-chloro-2- | 119.96 | 1.24 |
| | methoxyphenoxy)(phenyl)methyl]morpholine | | |
| | fumarate | | |
| 7 | (2S)-2-[(S)-(4-chloro-2- | 106.48 | 2.75 |
| | methoxyphenoxy)(phenyl)methyl]morpholine | | |
| | hydrobromide | | |
| 8 | (2S)-2-[(S)-(4-chloro-2- | 189.23 | 2.68 |
| | methoxyphenoxy)(phenyl)methyl]morpholine | | |
| | edisylate | | |
| 9 | (2S)-2-[(S)-(4-chloro-2- | 98.24 | 1.93 |
| | methoxyphenoxy)(phenyl)methyl]morpholine | | |
| | succinate | | |

### EXAMPLE 123

### Vapor Sorption Analysis of besylate, HCl, edisylate, and fumarate salts of (2S)-2-[(S)-(4-chloro-2-methoxyphenoxy)(phenyl)methyl]morpholine

The propensity of besylate, hydrochloride, edisylate and fumarate salts of (2S)-2-[(S)-(4-chloro-2-methoxyphenoxy)(phenyl)methyl]morpholine to absorb water vapor was studied at various relative humidities (RH). The besylate, hydrochloride, and edisylate salts were analysed using a VTI Corporation SGA-100 Symmetric Vapor Sorption Analyzer equipped with a CI Electronics Limited, CI MK2, 1 Gram Microbalance, an EdgeTech MODEL 2000 DEWPRIME DF DEWPOINT HYGROMETER, and an JULABO USA, Inc F25-HE Refrigerated and Heated Circulator. The following method was used:

| | |
|---|---|
| Drying Temp | 60° C. |
| Heating Rate | 5° C./min |
| Max Drying Time | 60 min |
| Equil Crit | 0.0100 wt % in 2 min |
| Expt Temp | 25° C. |
| Max Equil Time | 180 min |
| Equil Crit | 0.0100 wt % in 5 min |
| RH Steps (Besylate and | 10, 30, 50, 70, 90, 70, 50, 30, 10 |
| Hydrochloride Salts) | |
| RH Steps (Edisylate Salt) | 10, 30, 50, 70, 90, 70, |
| Data Logging Interval | 1.00 min or 0.0100 wt % |

The propensity of (2S)-2-[(S)-(4-chloro-2-methoxyphenoxy)(phenyl)methyl]morpholine edisylate to absorb water was similarly analyzed using a VTI Corporation SGA-100 Symmetric Vapor Sorption Analyzer equipped with a CAHN INSTRUMENTS INC, INC. D-200 Digital Recording Balance, an EdgeTech MODEL 2000 DEWPRIME DF DEWPOINT HYGROMETER, and a JULABO USA, Inc F25-HD Refrigerated and Heated Circulator. The following method was used:

| | |
|---|---|
| Drying Temp | 60° C. |
| Heating Rate | 5° C./min |
| Max Drying Time | 120 min |
| Equil Crit | 0.0100 wt % in 5 min |
| Expt Temp | 25° C. |
| Max Equil Time | 60 min |
| Equil Crit | 0.0100 wt % in 5 min |
| RH Steps | 10 to 90 to 10 by 10 |
| Data Logging Interval | 2.00 min or 0.0100 wt % |

The percent mass change at 90% relative humidity (RH) as compared to the original mass of the sample is reported in Table 12. The calculated moles of water uptake per total moles of the sample is reported in Table 12.

TABLE 12

| | | moles water |
|---|---|---|
| | | uptake per total |
| | % mass | moles of |
| | change at | sample at 90% |
| salt | 90% RH | RH |
| besylate | 0.64 | 0.17 |
| HCl | 3.8 | 0.77 |
| edisylate | 4.6 | 1.32 |
| fumaric | 2.8 | 0.69 |

### EXAMPLE 124

A single crystal structure of (2S)-2-[(S)-(4-chloro-2-methoxyphenoxy)(phenyl)methyl]morpholine besylate was solved from material made as in Example 110. The data were collected at room temperature using an APEX (Bruker-AXS) diffractometer. The structure was solved in the orthorhombic space group P2₁2₁2₁ with Z=4 (a=5.8086(18) Å, b=16.755(5) Å, c=49.587(15) Å. The structure solution contains two free-form besylate counterion pairs in the asymmetric unit. Hydrogen atoms were placed in calculated positions. The crystal structure shows that there is one besylate counter ion per (2S)-2-[(S)-(4-chloro-2-methoxyphenoxy)(phenyl)methyl]morpholine molecule.

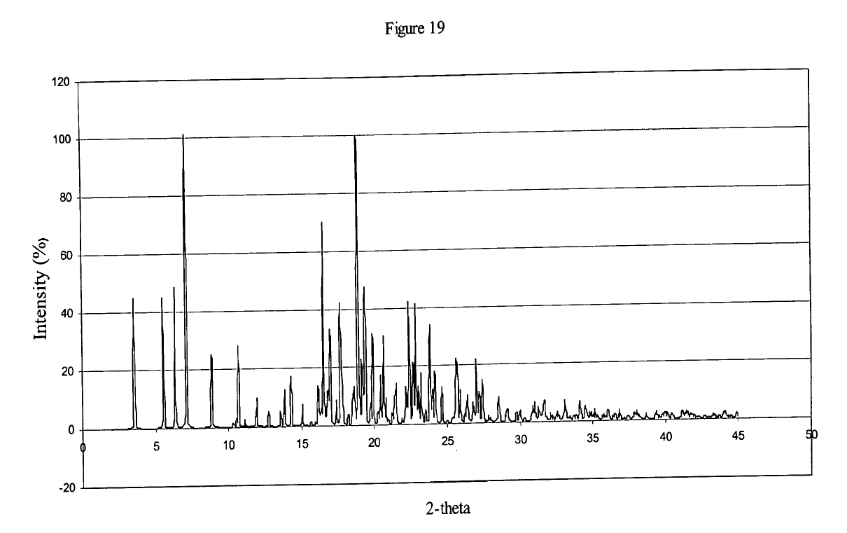

The crystal structure (not shown) is consistent with the molecular formula of (2S)-2-[(S)-(4-chloro-2-methoxyphenoxy)(phenyl)methyl]morpholine. The final model was refined to a goodness fit of 0.959 with R₁=0.0874 (I>2sigma(I)) and wR₂=0.1246(I>2sigma(1)). The absolute configuration of (2S)-2-[(S)-(4-chloro-2-methoxyphenoxy)(phenyl)methyl]morpholine besylate was determined from the flack parameter 0.0108 (esd 0.1279) vs 0.9798 (esd 0.1298) for the inverted structure. A calculated PXRD pattern was obtained from Material Studios software suite (FIG. 19). Summaries of the angle (2theta) values and intensity values (as a % of the value of the tallest peak) from the spectra are reported below in Table 13.

TABLE 13

| Angle (2theta) | Intensity % |
|---|---|
| 8.9° | 20.8 |
| 10.7° | 28.0 |
| 12.0° | 10.0 |
| 13.9° | 12.5 |
| 14.3° | 17.3 |
| 15.1° | 17.6 |
| 16.6° | 70.35 |
| 17.0° | 32.9 |
| 17.7° | 42.0 |
| 18.9° | 100 |
| 19.4° | 47.2 |
| 19.9° | 30.6 |
| 20.6° | 30.7 |
| 21.5° | 14.1 |
| 22.4° | 42.3 |
| 22.9° | 41.2 |
| 23.9° | 33.9 |
| 25.7° | 22.1 |
| 27.0° | 22.0 |
| 28.5° | 8.8 |
| 31.0° | 6.7 |

### EXAMPLE 124

Compounds of the present invention may be assayed for their ability to treat fibromylagia—like pain in a rat model of capsaicin-induced mechanical allodynia (e.g., Sluka, K A, (2002) J of Neuroscience, 22(13): 5687-5693). For example, a rat model of capsaicin-induced mechanical allodynia) was be carried out as follows:

On day 0, male Sprague-Dawley rats (˜150 g) in the dark cycle were placed in suspended wire-bottom cages and allowed to acclimate for 0.5 hour in a darkened, quiet room. The day 0 paw withdrawal threshold (PWT) was determined on the left hind paw by Von Frey hair assessment using the Dixon up and down method. After assessment, the plantar muscle of the right hind paw was injected with 100 μl capsaicin (0.25% (w/v) in 10% ethanol, 10% Tween 80, in sterile saline). On day 6 the PWT of the left hindpaw (contralateral from injection site) was determined for each animal. Animals from the day 6 prereads with PWT≦11.7 g were considered allodynic responders and were regrouped so that each cage had similar mean PWT values. On day 7, responders were dosed subcutaneously with 10 mg compound/kg body weight, or with vehicle alone. The vehicle was phosphate buffered saline containing 2% Cremophor® EL (BASF). The contralateral PWT values were determined at 1 hour after the single dose, with the investigator blinded to the dosing scheme.

For each animal, the day 6 PWT value was subtracted from the 1 hour PWT value to give a delta PWT value that represents the change in PWT due to the 1 hour drug treatment. In addition, the day 6 PWT was subtracted from the day 0 PWT to give the baseline window of allodynia present in each animal. To determine % inhibition of allodynia of each animal normalized for vehicle controls, the following formula was used: % Inhibition of Allodynia=100×[(Delta PWT(drug)−mean Delta PWT(vehicle))/(Baseline−mean Delta PWT(vehicle))].

The mean percent inhibition of allodynia values (for eight animals assayed for each compound) are shown in table 14. Values above 30% inhibition were found to be significant when compared to vehicle controls (evaluated by ANOVA and Dunnetts tests).

TABLE 14

| | % |
|---|---|
| Example Number | Inhibition |
| 37 | 80.3 |
| 38 | 59 |
| 41 | 21.1 |
| 45 | 54.6 |
| 46 | 46.1 |
| 48 | 6.5 |
| 52 | 7 |
| 56 | 35.6 |
| 62 | 40.5 |
| 70 | 27.3 |
| 79 | 99.5 |
| 80 | 40 |
| 87 | 9.3 |
| 89 | 27.8 |
| 93 | 39 |
| 102 | 59.5 |
| 105 | 13.7 |
| 110 | 71.1 |
| (2S)-2-[(1S)-(2- | 17 |
| ethoxyphenoxy)(phenyl)methyl]morpholine | |

1-23. (canceled)

24. A compound (2S)-2-[(1S)-(4-chloro-2-methoxyphenoxy)(pyridin-2-yl)methyl]morpholine, or a pharmaceutically acceptable salt thereof.

25. A compound of claim 24 which is (2S)-2-[(1S)-(4-chloro-2-methoxyphenoxy)(pyridin-2-yl)methyl]morpholine.

26. A compound of claim 24 which is (2S)-2-[(1S)-(4-chloro-2-methoxyphenoxy)(pyridin-2-yl)methyl]morpholine hydrochloride.

27. A method of treating a disorder in a mammal, wherein the disorder is selected from urinary disorders, genuine stress incontinence, stress urinary incontinence, pain, premature ejaculation, depression, generalised anxiety disorder, ADHD and fibromyalgia, which method comprises administering to a mammal in need of such treatment a therapeutically effective amount of a compound (2S)-2-[(1S)-(4-chloro-2-methoxyphenoxy)(pyridin-2-yl)methyl]morpholine, or a pharmaceutically acceptable salt thereof.

28. A method of claim 27 wherein the compound is (2S)-2-[(1S)-(4-chloro-2-methoxyphenoxy)(pyridin-2-yl)methyl]morpholine hydrochloride.

29. The method of treating pain of claim 27 wherein the pain is selected from the group of neuropathic pain, inflammatory pain, musculo-skeletal pain, central pain, heart and vascular pain, visceral pain, head pain or orofacial pain.

30. The method of treating pain of claim 27 wherein the pain is inflammatory pain associated with rheumatoid arthritis, osteoarthritis or inflammatory bowel disease.

31. The method of treating pain of claim 27 wherein the pain is neuropathic pain.

32. The method of treating fibromyalgia of claim 27 comprising administering to a mammal in need of such treatment a therapeutically effective amount of (2S)-2-[(1S)-(4-chloro-2-methoxyphenoxy)(pyridin-2-yl)methyl]morpholine hydrochloride.

33. The method of treating a disorder of claim 27 wherein the mammal is a human.

34. The method of treating a disorder of claim 33 comprising an oral or parenteral administration of a compound (2S)-2-[(1S)-(4-chloro-2-methoxyphenoxy)(pyridin-2-yl)methyl]morpholine, or a pharmaceutically acceptable salt thereof, at a daily dosage level of from 10 to 500 mg.

35. The method of treating a disorder of claim 33 comprising an oral or parenteral administration of a compound (2S)-2-[(1S)-(4-chloro-2-methoxyphenoxy)(pyridin-2-yl)methyl]morpholine, or a pharmaceutically acceptable salt thereof, at a daily dosage level of from 0.01 to 30 mg/kg.

36. The method of treating a disorder of claim 33 wherein the compound is (2S)-2-[(1S)-(4-chloro-2-methoxyphenoxy)(pyridin-2-yl)methyl]morpholine hydrochloride.

37. A pharmaceutical composition comprising a therapeutically effective amount of (2S)-2-[(1S)-(4-chloro-2-methoxyphenoxy)(pyridin-2-yl)methyl]morpholine, or a pharmaceutically acceptable salt thereof, and a pharmaceutically acceptable excipient, diluent or carrier.

38. A pharmaceutical composition of claim 37 comprising a therapeutically effective amount of (2S)-2-[(1S)-(4-chloro-2-methoxyphenoxy)(pyridin-2-yl)methyl]morpholine hydrochloride and a pharmaceutically acceptable excipient, diluent or carrier.

39. A pharmaceutical composition of claim 37 comprising from 5 mg to 250 mg of (2S)-2-[(1S)-(4-chloro-2-methoxyphenoxy)(pyridin-2-yl)methyl]morpholine, or a pharmaceutically acceptable salt thereof, and a pharmaceutically acceptable excipient, diluent or carrier in a tablet or capsule.

40. A pharmaceutical composition of claim 37 comprising from about 0.01 mg to 500 mg of (2S)-2-[(1S)-(4-chloro-2-methoxyphenoxy)(pyridin-2-yl)methyl]morpholine, or a pharmaceutically acceptable salt thereof, and a pharmaceutically acceptable excipient, diluent or carrier in a tablet formulation.
